(19) United States
(12) Patent Application Publication
(10) Pub. No.: US 2010/0247381 A1
(43) Pub. Date: Sep. 30, 2010

(54) PROCESSES AND APPLICATIONS OF CARBON NANOTUBE DISPERSIONS

(75) Inventors: Arjun G. Yodh, Merion, PA (US); Mohammad F. Islam, Pittsburgh, PA (US); Alan T. Johnson, JR., Philadelphia, PA (US); Danvers E. Johnston, Philadelphia, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: THE TRUSTEES OF THE UNIVERSITY OF PENNSYLVANIA, Philadelphia, PA (US)

(21) Appl. No.: 12/792,963

(22) Filed: Jun. 3, 2010

Related U.S. Application Data
(63) Continuation of application No. 11/145,627, filed on Jun. 6, 2005.
Application No. 11/145,627 is a continuation-in-part of application No. 10/526,941, filed on Sep. 8, 2005.
(60) Provisional application No. 60/409,821, filed on Sep. 10, 2002.
(60) Provisional application No. 60/419,882, filed on Oct. 18, 2002.

(51) Int. Cl. G01N 33/00; D01F 9/12; C09K 5/00; H01B 1/04; C07C 211/02; C07C 53/00; C07C 11/00; B05D 5/00; B32B 3/10; H05K 1/00; G21G 4/00

(52) U.S. Cl. 422/68.1; 423/447.2; 252/74; 252/511; 423/447.1; 564/463; 562/512; 585/16; 427/256; 428/195.1; 428/172; 174/250; 250/493.1; 977/742; 977/750; 977/842

(57) ABSTRACT

Disclosed are copolymers of carbon nanotubes, as well as processes and applications of carbon nanotube dispersions. Carbon nanotube emulsions and related technology are also disclosed. The controlled deposition of carbon nanotubes on substrates is also provided. Methods of purifying single-walled carbon nanotubes are also provided. Devices made according to the disclosed methods are further described herein.

### CROSS REFERENCE TO RELATED APPLICATIONS

This application is a continuation of U.S. Ser. No. 11/145,627, filed Jun. 6, 2005, which application is a continuation-in-part application of U.S. Ser. No. 10/526,941, filed Mar. 8, 2005, which is the National Stage of International Application No. PCT/US2003/016086, filed May 21, 2003, which claims the benefit of U.S. Provisional Application No. 60/409,821, filed Sep. 10, 2002, and U.S. Provisional Application No. 60/419,882, filed Oct. 18, 2002, the disclosures of which are incorporated herein by reference in their entireties for any and all purposes. This application also claims the benefit of U.S. Provisional Application No. 60/576,940, filed Jun. 4, 2004, the disclosure of which is incorporated herein by reference in its entirety.

### GOVERNMENT RIGHTS

The work leading to the disclosed invention was funded in whole or in part with Federal funds from the National Science Foundation and the National Aeronautic and Space Administration. The Government may have certain rights in the invention under NSF contract DMR0079909 and NASA Grant NAGS-2172.

### FIELD OF THE INVENTION

The present invention is related to the field of carbon nanotubes. The present invention is also related to dispersions containing carbon nanotubes. In addition, the present invention is related to the field of materials and devices that contain carbon nanotubes. The present invention is also related to processes and applications of carbon nanotube dispersions.

### BACKGROUND OF THE INVENTION

Carbon nanotubes are tiny fullerene-related structures of graphene cylinders having nanoscale diameters from about 0.7 to about 50 nanometers (“nm”) and microscopic lengths from about 0.1 to about 20 microns (“μm”). Carbon nanotubes are readily synthesized catalytically from hot carbon vapor or by thermal decomposition of a carbon-containing gas or liquid. Different synthetic methods yield nanotubes with one or several nested cylinders and different degrees of perfection. Various morphologies, tube shape, atomic conformations, and chemical compositions lead to a variety of uses. Chemical reactions inside or on the tube surface can be exploited for energy storage and drug delivery. The mechanical, electronic and thermal properties of carbon nanotubes enable a broad spectrum of applications including inter alia molecular electronics, nucleic acid and proteomic sequencing, high-strength composites, solar heat generation, energy storage and heat transfer.

The synthesis, characterization and useful applications of carbon nanotubes has been a fertile area of research for over twelve years, beginning with the discovery of multi-wall carbon nanotubes in 1991 by S. Iijima, as reported in Helical Microtubules of Graphitic Carbon, Nature 354, 56 (1991). Shortly thereafter, several groups reported on the electrically conductive properties of carbon nanotubes in Are Fullerene Tubules Metallic?, J. W. Mintmire et al., Phys. Rev. Lett. 68, 631 (1992), in New One-Dimensional Conductors—Graphitic Microtubules, N. Hamada et al., Phys. Rev. Lett. 68, 1579 (1992), and in Electronic Structure of Graphene Tubules Based on C₆₀, R. Saito et al., Phys. Rev. B 46, 1804 (1992). In 1993, Overney et al., reported in the mechanical properties of carbon nanotubes in Structural Rigidity and Low Frequency Vibrational Modes of Long Carbon Tubules, Phys. D 27, 93 (1993). In the same year, S. Iijima et al. reported their synthesis of single-wall nanotubes in Single-Shell Carbon Nanotubes of 1-nm Diameter, Nature, 363, 603 (1993), and Bethune et al. reported on the synthesis of single wall carbon nanotubes in Cobalt-Catalysed Growth of Carbon Nanotubes with Single-Atomic-Layer Walls, Nature, 363, 605 (1993).

Reports of the use of carbon nanotubes in a variety of applications became more frequent as their preparation became more routine. For example, Rinzler et al. reported the use of nanotubes as field emitters in Unraveling Nanotubes: Field Emission from an Atomic Wire, Science 269, 1550 (1995). In 1996, ropes of single-wall nanotubes were reported in Crystalline Ropes of Metallic Carbon Nanotubes by A. Thess et al., Science 273, 483 (1996).

The quantum conductance of carbon nanotubes was reported in 1997 by Tans et al. in Individual Single-Wall Carbon Nanotubes as Quantum Wires, Nature, 386, 474 (1997). That same year, hydrogen storage in nanotubes was reported by Dillon et al. in Storage of Hydrogen in Single-Walled Carbon Nanotubes, Nature, 386, 377 (1997). The chemical vapor deposition (CVD) synthesis of aligned nanotube films was reported in Synthesis of Large Arrays of Well-Aligned Carbon Nanotubes on Glass, Z F Ren et al., Science, 282, 1105 (1998), and the synthesis of “nanotube peapods” was reported by Smith et al. in Encapsulated C₆₀ in Carbon Nanotubes, Nature 396, 323 (1998).

One of the more interesting properties of carbon nanotubes is their unusually high thermal conductivity, which can be useful for preparing materials for managing heat in a variety of useful systems and devices. For example, S. Berber et al. reported in 2000 Unusually High Thermal Conductivity of Carbon Nanotubes, Phys. Rev. Lett. 84, 4613 (2000). Another interesting property is their unusually high strength of macroscopically aligned nanotubes, as reported in Macroscopic Fibers and Ribbons of Oriented Carbon Nanotubes, B. Vigolo et al., Science 290, 1331 (2000).

In 2001, the integration of carbon nanotubes for logic circuits was reported in Engineering Carbon Nanotubes and Nanotube Circuits Using Electrical Breakdown, P. C. Collins et al., Science 292, 706 (2001). The intrinsic superconductivity of carbon nanotubes was also reported that year by M. Kociak et al., Phys. Rev. Lett. 86, 2416 (2001).

Recently in Molecular Design of Strong Single-wall Carbon Nanotube/Polyelectrolyte Multilayer Composites, Nature Materials, 1(3):190-194 (2002), Mamedov et al. described the preparation of a layered polymer/carbon nanotube composite made by attaching chemical groups to the nanotubes that form bonds with the polymer when the material is heated, or treated chemically.

As used herein, the term “carbon nanotube” refers to a variety of hollow, partially filled and filled forms of rod-shaped and toroidal-shaped hexagonal graphite layers. Examples of hollow carbon nanotubes include single-wall carbon nanotubes, multi-wall carbon nanotubes, carbon nanotoroids, branched carbon nanotubes, armchair carbon nanotubes, zigzag carbon nanotubes, as well as chiral carbon nanotubes. Filled carbon nanotubes include carbon nanotubes containing various other atomic, molecular, or atomic and molecular species within its interior. Examples include nanorods, which are nanotubes filled with other materials, like oxides, carbides, or nitrides. Examples of filled carbon nanotubes include carbon nanofibers having carbon within its interior. Carbon nanotubes that have hollow interiors have also be opened and filled with non-carbon materials using wet chemistry techniques to provided filled carbon nanotubes.

A single-walled carbon nanotube (SWNT) can be imagined as a rolled-up rectangular strip of hexagonal graphite monolayers. The short side of the rectangle becomes the tube diameter and therefore is “quantized” by the requirement that the rolled-up tube must have a continuous lattice structure. The rectangle is typically oriented with respect to the flat hexagonal lattice to allow a finite number of roll-up choices. Two of these correspond to high symmetry SWNTs; in “zigzag” nanotubes, some of the C—C bonds lie parallel to the tube axis, while in “armchair” nanotubes, some bonds are perpendicular to the axis. Chiral nanotubes have a left- or right-handed screw axis, like DNA. Carbon nanotubes can also be nested together, one inside another to form so-called “nanocables”. Carbon nanotubes can also have one end wider than the other to form so-called “nanocones”. Carbon nanotubes in which the ends attach to each other to form a torus shape are commonly referred to as carbon “nanotoroids”.

The allowed electron wave functions of SWNTs are different than those of an infinite two-dimensional system of hexagonal graphite monolayers. In contrast the structure of a hexagonal graphite monolayer, the rolling operation imposes periodic boundary conditions for propagation around the circumference. This gives rise to a different electronic band structure for different symmetries of carbon nanotubes. As a consequence, SWNTs can be either metallic or insulating, with bandgaps in the latter typically ranging from a few milli-electron volts to about one electron volt.

Carbon nanotubes can also be used bundled together or isolated. Nanotube bundles of many SWNTs with similar diameters are able to self-organize (order, i.e., “crystallize”) during growth into a triangular lattice. Nanotubes may be isolated on surfaces, isolated in dilute fluid dispersions, and isolated in composite materials and devices. Bulk materials containing porous mats of nanotubes can be prepared from entangled bundles of carbon nanotubes.

SWNT bundles are carbon-based materials into which heteroatoms or molecules can be inserted and removed. It is known that the proper choice of heteroatoms or molecules (alkali metals, halogen or acid molecules) can transform an insulating polymeric host into a doped semiconductor or even a metal, an example being sodium-doped polyacetylene. In a similar fashion, insulating molecular fullerene solids become superconducting upon addition of three alkali ions per molecule. Likewise, reversible insertion in graphite and SWNT bundles can be exploited for energy storage applications such as rechargeable batteries (e.g., Li-doped SWNT bundles) and “hydrogen containers” for use in hydrogen-burning vehicles.

In view of the many fascinating novel electronic, thermal and mechanical properties of carbon nanotubes, many applications that will take advantage of these properties will require large-scale manipulations of stable solutions of carbon nanotubes having high weight fractions of individual carbon nanotubes. For example, dispersions of individual carbon nanotubes will enable the use of a variety of solution-phase purification and separation methodologies. Accordingly, the preparation of high nanotube weight fraction solutions will facilitate a variety of processing steps performed on, and with, carbon nanotubes. Such processing steps include inter alia chemical derivatization, controlled deposition, microfluidic processes, fabrication of nanotube-based fibers, preparation of coatings and composite materials, as well as the fabrication of a variety of electronic, optical, micromechanical and microfluidic devices. Furthermore, high volume fraction nanotube solubilization will bring nanotube science into better contact with fundamental research on interactions and self-assembly in complex fluids. Unfortunately, as a result of the substantial van der Walls attractive forces between them, nanotubes readily aggregate and are difficult to keep individually dispersed in solution.

Some progress has been made towards solubilization of carbon nanotubes in organic and aqueous media. Dissolution in organic solvents has been reported with bare SWNT fragments (e.g., 100 to 300 nm length) by Bahr et al., Chem. Commun, 2, 193, (2001) and by Ausman et al., J. Phys. Chem. B 104, 8911 (2000). Likewise, the dissolution of chemically-modified SWNTs has been reported by Chen et al., Science, 282, 95 (1998) and by Chen et al., J. Am. Chem. Soc. 123, 3838 (2001). Dissolution in water, important because of potential biomedical applications and biophysical schemes, has also been reported by Liu et al., Science 280, 1253 (1998), Bandow et al., J. Phys. Chem. B 101. 8839 (1997), Duesberg et al., Chem. Commun. 3, 453 (1998), Shelimov et al., Chem. Phys. Lett. 282, 429 (1998), and Bandyopadhyaya et al., Nano Letters 2, 25-28 (2002). Dissolution of carbon nanotubes by polymer wrapping has been reported by O'Connell et al., Chem. Phys. Lett. 342, 265 (2001) and by Star et al., Agnew, Chem. Int. ed. 41, 2508 (2002).

Dissolution by chemical modification of the carbon nanotubes has been reported by Sano et al., Langmuir, 17, 5125 (2001), Nakashima et al., Chem. Lett. P. 638 (2002), and by Pompeo et al., Nanoletters 2, 369 (2002). Generally, the chemically modified carbon nanotubes are less desirable because their band structures can differ from the unmodified nanotubes. As well, chemically modified carbon nanotubes tend to be shorter than unmodified nanotubes. Indeed, carbon fibers having lengths greater than about 500 nm are desirable for introducing anisotropic properties in composite materials, as reported by Halpin et al. in Polymer Eng. Sci. 16, 344 (1976). Unfortunately, tube breakage typically accompanies preparation of dispersions of carbon nanotubes longer than about 500 nm. Thus, there remains the problem of providing carbon nanotube dispersions that do not require chemical modification and which provide high volume fractions of long carbon nanotubes with minimal breakage.

Applications for carbon nanotubes generally fall into two categories: those requiring isolated carbon nanotubes and those requiring ensembles of carbon nanotubes. In applications using ensembles of carbon nanotubes, especially for composite materials, a high degree of nanotube alignment is desired. Aligning carbon nanotubes has been difficult, however. With few exceptions (Jin et al., Appl. Phys. Lett. 73, 1197 (1998) and Hadjiev et al., Appl. Phys. Lett. 78, 3193 (2001)), the vast majority of solution- and solid-phase mixtures are isotropic, as reported by Schadler et al., Appl. Phys. Lett. 73, 3842 (1998), Bower et al., Appl. Phys. Lett. 74, 3317 (1999), Sandler et al., Polymer 40, 5967 (1999), Andrews et al., Appl. Phys. Lett. 75, 1329 (1999), and Qian et al., Appl. Phys. Lett. 76, 2868 (2000). Accordingly, stable nematic-like phases of carbon nanotubes, especially of the SWNT variety, have been elusive. Thus, there also remains the problem of providing oriented ensembles of carbon nanotubes.

Several groups have attempted to covalently bind functionalized CNT with polymer. Sun and coworkers reported the covalent bonding of carbon nanotubes with poly (propionylethylenimine-co-ethylenimine) and poly [(vinyl acetate)-co-(vinyl alcohol)] (J. E. Riggs, Z. Guo, D. L. Carroll, Y. P. Sun, J. Am. Chem. Soc. 122, 5879 (2000)). This group also reported to functionalize multi-walled carbon nanotubes with a polystyrene copolymer (D. E. Hill, Y. Lin, A. M. Rao, L. F. Allard, Y. P. Sun, Macromolecules 35, 9466 (2002)). Recently, Haddon's group fabricated a water-soluble single-walled carbon nanotube-poly (m-aminobenzene sulfonic acid) graft copolymer (B. Zhao, H. Hu, R. C. Haddon, Adv. Funct. Mater. 14, 71 (2004)). These publications appear to be limited to polymers grafted to a carbon nanotube, e.g., a nanotube is used as side arm graft or a side block part of a polymer, and not along the main chain backbone. However, the chain structure of the carbon nanotube appears to be important for preparing composite materials and nano-fibers having strong mechanical and high conductive properties. Accordingly, the development of polymers and copolymers comprising a chain structure of carbon nanotubes along the chain backbone is presently needed.

There is also a present need to prepare emulsions containing carbon nanotubes in the emulsified particle phase, in the continuous fluid phase, or both. Emulsified carbon nanotubes may be useful, for example, in preparing composite and hybrid materials having nanotubes dispersed throughout the matrix phase. CNT-polyaniline hybrid materials have been obtained by emulsion polymerization (Chan, et al., European Polymer Journal 38, 2497 (2002)). Bahr's group added CNT to high concentration PVA aqueous emulsion for preparing low percolation threshold conducting composite materials (Bahr, et. al., Adv. Mater. 16, 150 (2004)). Thus, there remains a continuing need to emulsify nanotubes, for example, in preparing composite and hybrid materials.

There is also a present need to be able to controllably deposit carbon nanotubes on a substrate. Liu, et al., (Chem. Phys. Lett. 303, 125 (1999)) dispersed CNT in DMF todeposited CNT is confined in a controlled area. Rao, et al., (Nature 425, 36 (2003)) used gold as the substrate and graft mercaptan agents to grow positive charged, negative charged and non-polar molecular monolayers on top of the gold surface. Lay, et al., Nano. Lett. 4, 603 (2004), dispersed CNT with SDS solution and flow aligned the CNTs during the drying process. In view of these publications, there still remains the need to controllably deposit well isolated, i.e., single, carbon nanotubes, on substrates. This will be particularly useful in preparing CNT-based circuits and sensors.

There is also a present need to control the length of nanotubes, for example, by fractionating solutions of nanotubes that are polydisperse in length. Ji, et al., Chem. Phys. Lett. 352, 328 (2002) centrifuged organic DMF solutions of PVDF-dispersed MWNT dispersions with different speeds to separate nanotubes according to their aspect ratios. Sun, et al., Langmuir 19, 7084 (2003) functionalized SWNTs with a diamine-terminated oligomeric poly(ethylene glycol) and fractionated the SWNTs accordingly to preferential solubilization of smaller diameter SWNTs. Zheng, et al., Science 302, 1545 (2003) separated SWNTs by diameter by dispersing the SWNTs with DNA and using anion exchange chromatography. Accordingly, there still remains the need to efficiently separate CNTs according to length in aqueous solutions.

### SUMMARY OF THE INVENTION

The present inventors have discovered that a particular class of surfactants is capable of providing stable dispersions of high concentrations of carbon nanotubes in aqueous media without requiring the aforesaid techniques of chemical modification or polymer wrapping. In a first aspect of the present invention, there are provided dispersions including an aqueous medium, carbon nanotubes, and at least one surfactant, the surfactant having an aromatic group, an alkyl group having from about 4 to about 30 carbon atoms, and a charged head group.

The present inventors have also discovered that a particular class of surfactants when used with an ultrasonication process is capable of providing stable dispersions of carbon nanotubes having reduced breakage of the carbon nanotubes. Thus, in a second aspect of the present invention, there are provided methods of preparing dispersions of carbon nanotubes, in which the methods include mixing an aqueous medium, carbon nanotubes, and surfactant in a low-power, high-frequency bath sonicator. In this aspect of the invention, the surfactant includes an alkyl group having from about 4 to about 30 carbon atoms, an aromatic group, and a charged head group.

Within additional aspects of the invention there are provided compositions of carbon nanotubes that can be used in a variety of applications. In this aspect of the invention, there are provided compositions including carbon nanotubes and surfactant, wherein the surfactant has an alkyl group having from about 4 to about 30 carbon atoms, an aromatic group, and a charged head group.

In another aspect of the invention, there are provided composite materials containing carbon nanotubes. In this aspect of the invention, the composite materials have a solid matrix and carbon nanotubes and surfactant dispersed within the solid matrix, the surfactant having an alkyl group having from about 4 to about 30 carbon atoms, an aromatic group, and a head group.

In a related aspect of the invention, there are provided methods of preparing composite materials using the carbon nanotube dispersions provided herein. In this aspect of the present invention, there methods include dispersing carbon nanotubes and surfactant in a hardenable matrix precursor, and hardening the precursor. In these methods, the surfactant includes an alkyl group having from about 4 to about 30 carbon atoms, an aromatic group, and a head group.

In another aspect of the invention, there are provided assemblies of carbon nanotubes. In this aspect of the invention, the assemblies include a substrate, and carbon nanotubes and surfactant adjacent to the substrate. In this aspect of the invention, the surfactant has an alkyl group having from about 4 to about 30 carbon atoms, an aromatic group, and a charged head group.

In another aspect of the invention, there are provided methods of assembling carbon nanotubes on a substrate. In this aspect of the invention, the methods of assembling carbon nanotubes include contacting dispersions including an aqueous medium, carbon nanotubes and surfactant to a substrate. In this aspect of the invention, the surfactant includes an alkyl group having from about 4 to about 30 carbon atoms, an aromatic group, and a charged head group. These methods can be used, for example, in providing solid media for use in detecting chemical and biological substances. Thus, in a related aspect of the present invention, there are provided solid media having a substrate for receiving chemical compounds, biological materials, or both biological materials and chemical compounds for use in detecting chemical and biological substances. In this aspect of the invention the substrate includes carbon nanotubes and surfactant adsorbed thereon, the surfactant comprising an alkyl group having from about 4 to about 30 carbon atoms, an aromatic group, and a charged head group.

The present inventors have also discovered that the dispersed carbon nanotubes of the present invention can also be used to prepare nematic nanotube gels. In this aspect of the invention, the methods of preparing nematic nanotube gels include:

providing a dispersion of carbon nanotubes, solvent, gel precursor, and surfactant, the surfactant including an alkyl group having from about 4 to about 30 carbon atoms, an aromatic group, and a charged head group;

gelling at least a portion of the gel precursor to form a gel; and

subjecting the dispersion, the gel, or both the dispersion and the gel to an orienting field, the orienting field giving rise to a nematic orientation of said carbon nanotubes.

The present inventors have also discovered compositions containing carbon nanotubes and gel precursors. In this aspect of the invention, the composition includes carbon nanotubes, gel precursor, and surfactant, the surfactant having an alkyl group having from about 4 to about 30 carbon atoms, an aromatic group, and a charged head group.

In another aspect of the present invention, there are provided copolymers, comprising a plurality of end-linked single-wall carbon nanotubes.

In yet other aspects, the present invention provides copolymers, comprising a plurality of covalently-bonded repeating groups, at least a portion of said repeating groups comprising functionalized single-wall carbon nanotubes.

Also provided are methods of the present invention that comprise opening both ends of a carbon nanotube; providing at least one covalently-bound functional group to each of said ends; and covalently bonding at least one monomeric compound to said at least one covalently-bound functional group.

In another aspect of the present invention, there are provided polymers comprising a chain structure of a plurality of covalently-bonded open-ended carbon nanotubes.

In yet other aspects of the present invention, there are provided methods, comprising providing a T-channel microfluidic device, comprising: a microchannel comprising an inlet, a junction and an exit; a first fluid conduit capable of transporting a first fluid into the microchannel at said inlet; a second fluid conduit, said second conduit capable of transporting a second fluid into the microchannel at said junction; fluidically transporting said first fluid from said first conduit into said microchannel; fluidically transporting said second fluid from said first conduit into said microchannel, and; forming a dispersed phase of said second fluid in a continuous phase of said first fluid in the microchannel, wherein said first fluid, said second fluid, or both, comprise an aqueous dispersion of carbon nanotubes.

The present invention also provides methods comprising providing a patterned substrate comprising a polymer layer and exposed surface features; bonding charged linker molecules, linker molecules capable of being charged, or both, to said exposed surface features; removing said polymer layer; optionally charging the linker molecules capable of being charged; and bonding charged carbon nanotubes to the charged linker molecules, wherein the charge of the charged carbon nanotubes is opposite the charge of the charged linker molecules bonded to the exposed surface features.

In other aspects, there are provided substrates a surface feature comprising one or more charged linker molecules; and a charged carbon nanotube controllably deposited on said charged linker molecules, wherein the charge of the charged carbon nanotube is opposite the charge of the charged linker molecules.

The present invention also provides devices, comprising a substrate fluidically sealed to a microfluidic assembly, said substrate comprising negatively charged carbon nanotubes adsorbed on one or more negatively charged regions on a surface of the substrate; the microfluidic assembly comprising one or more contacting regions adjacently positioned to the substrate for controllably contacting one or more molecular components to said carbon nanotubes; one or more target fluid conduits capable of supplying one or more target fluids comprising one or more analytes; one or more detecting molecule conduits capable of supplying one or more detecting molecules for detecting said analytes in the target fluids; one or more valves capable of directing said target fluids and said detecting molecules into said contacting regions; and optionally one or more exit conduits.

Additional aspects of the present invention also provide processes that include providing an aqueous carbon nanotube dispersion comprising water and individual, dispersed, carbon nanotubes; and chromatographically separating the carbon nanotubes.

Other aspects of the present invention will be apparent to those skilled in the art in view of the detailed description and drawings of the invention as provided herein.

### BRIEF DESCRIPTION OF THE DRAWINGS

The invention may be understood by reference to the following description taken in conjunction with the accompanying drawings, in which:

FIG. 1 shows vials containing aqueous dispersions of SWNTs at 5 mg/ml after two weeks of incubation at room temperature with various surfactants. (A) SDS-HiPCO; (B) TX100-HiPCO; (C) NaDDBS-HiPCO. Carbon nanotube dispersions prepared with NaDDBS surfactant (C) are homogeneous whereas dispersions prepared with SDS (A) and TX100 (B) coagulate, forming a mass of aggregated nanotubes at the bottom of the vial.

FIG. 2 shows a tapping mode AFM image of TX100 stabilized laser-oven produced single-walled carbon nanotubes on a silicon surface. A dispersion of the carbon nanotubes was prepared at a concentration of 0.1 mg/ml by bath sonicator.

FIG. 3 shows the length and diameter distribution of HiPCO carbon nanotubes in various attempted dispersions. Data obtained from AFM images like the one in FIG. 2, after dispersion by bath sonicator and stabilized using three different surfactants. For original dispersion concentrations greater than 0.1 mg/ml, the dispersions were rapidly diluted to 0.1 mg/ml, and then spread over a silicon wafer for nanotube length distribution measurements using AFM. The contributions of nanotubes having lengths less than 50 nm are not reflected in these nanotube length distributions because of the limitation of the lateral resolution of these measurements. (a) The number fraction of single nanotubes in a NaDDBS-HiPCO dispersion prepared at 0.1 mg/ml was about 74±5 percent. (b) The number fraction of single nanotubes in a NaDDBS-HiPCO dispersion prepared at 20 mg/ml was about 63±5 percent. (c) The number fraction of single nanotubes in a NaDDBS-HiPCO dispersion prepared at 10 mg/ml was about 61±5 percent. (d) Repeat of (c) after sitting for one month (about 55±5 percent, based on number, single nanotubes). (e) The number fraction of single nanotubes in a SDS-HiPCO dispersion prepared at 0.1 mg/ml was 16±2 percent. (f) The number fraction of single nanotubes in a TritonX-100-HiPCO dispersion prepared at 0.1 mg/ml was 36±3 percent.

FIG. 4 shows a schematic representation of how surfactant may adsorb onto the exterior surface of a tube. It is speculated that the alkyl chain groups of a surfactant molecule adsorb flat along the length of the tube rather than bend around the circumference. NaDDBS and TX100 disperse the nanotubes better than SDS because of their aromatic groups. NaDDBS also disperses carbon nanotubes better than TX100 because of its chargeable head group and slightly longer alkyl chain.

FIG. 5 shows the length and diameter distribution of 0.1 mg/ml laser-oven single-walled nanotube dispersions using NaDDBS as the surfactant and produced by tip and bath sonicators. (a) The low-power bath sonication method provided a high yield (90±5 percent) of single (individual) carbon nanotubes; many individual carbon nanotubes had lengths longer than 400 nm post sonication, Lₘₑₐₙ was about 516±286 nm. (b) The tip-sonication technique gave significantly lower yield (50±5 percent) and fragmented more nanotubes than in (a); Only a few nanotubes having lengths larger than 400 nm were observed, and the mean length, Lₘₑₐₙ was about 267±126 nm.

FIG. 6 shows a schematic of a NIPA gel structure (homogeneous) after NIPA monomer is polymerized in the presence of a gel initiator and cross-linker at 296 K.

FIG. 7 shows capillary nanotubes containing SWNT-NIPA gels before and after subjecting the gels to an orienting pressure field, which causes the gels to shrink. Capillary nanotubes containing initial nanotube concentrations of (a) 0.78 mg/ml and (b) 0.23 mg/ml appeared dark because the carbon nanotubes absorb light. A NIPA gel containing NaDDBS surfactant and no carbon nanotubes (c) was prepared to study the effects of the presence of the carbon nanotubes on the gel's shrinking. Here, the NIPA gel appears to shrink almost the same ratio whether or not the carbon nanotubes are present.

FIG. 8 shows birefringence images of a carbon nanotube gel with initial nanotube concentration of 0.78 mg/ml, observed at different angles after sitting for four days. Images were taken with a fixed microscope bulb intensity and video gain and offset. Maximum birefringence was found when the samples was oriented 45 degrees with respect to the input polarizer pass axis. Liquid crystal like defects were observed near the edges of the nanotube gel, which are clearly visible when the gel was in vertical (0 degree) or horizontal (90 degrees) orientations. Greater nanotube alignment is observed near the gel edges. Evidently the director tends to align near the walls, perhaps as a result of boundary effects imposed by the walls. The central dark regions appear to be disordered in this figure; but when rotated on an axis coincident with the short edge of the sample by about 30 degrees, the central dark regions became bright, indicating that the central dark regions were at least partially ordered (not shown).

FIG. 9 shows a summary of the effects of time and nanotube concentration on the alignment of nanotubes in NIPA gels. The bulb intensity and video gain offset were kept fixed. All of the samples were isotropic before shrinking. Birefringence was observed after the samples were shrunk upon subjecting them to an orienting pressure field.

FIG. 10 shows capillary nanotubes with SWNTs-NIPA gel placed inside a vacuum jar, from which water slowly migrates out of the gel upon application of a pressure field using a vacuum pump.

FIG. 11 shows images of carbon nanotubes inside NIPA gels that were isotropic before water extrusion at 0.46 mg/ml (a). As water was extruded from the gel, the carbon nanotubes began aligning along the flow direction of water and the gel became birefringent (b). At high enough initial concentration of nanotubes (0.46 mg/ml) in gel and after significant extrusion of water, some of the aligned nanotubes formed small ropes with the gel (c). The image (c) is a bright-field image at a higher magnification compared to (a) and (b).

FIG. 12 shows carbon nanotubes that were aligned inside a NIPA gel using a nine Tesla magnetic field. Also observed are nanotube needles arising from the end-to-end chaining of multiple nanotubes.

FIG. 13(a) is an illustration of an embodiment of the present invention of a copolymer composed of a plurality of single wall carbon nanotubes.

FIG. 13(b) is an illustration of an embodiment of the present invention of a difunctionalized dual open-end SWNT monomer.

FIG. 14 presents FTIR absorption data (in the order from the bottom data curve to the top data curve): pristine CNT; pristine NH₂—PEG-NH₂; a physical mixture of CNT and NH₂—PEG-NH₂; sedimentation 1 of amidation of CNT and NH₂—PEG-NH₂; and sedimentation 1 of amidation of CNT and NH₂—PEG-NH₂.

FIG. 15 is a schematic illustration of a microfluidic T channel used in preparing CNT emulsions of the present invention.

FIG. 16 provides schematic illustrations of embodiments of emulsions of the present invention: (a) (water+NaDDBS+CNT)/monomer inverse emulsion; (b) monomer/(water+NaDDBS+CNT) direct emulsion; (c) (water+high weight % NaDDBS+CNT)/(water+low weight % NaDDBS).

FIG. 17 is an image of an embodiment of the emulsions of the present invention.

FIG. 18 is a schematic illustration of a process of the present invention for depositing carbon nanotubes on a substrate.

FIG. 19(a) is an atomic force micrograph of ˜200 nm wide channels etched in a PMMA layer on top of a silicon wafer. The exposed silicon wafer in the channels is oxidized.

FIG. 19(b) is an atomic force micrograph of carbon nanotubes covering an oxidized silicon wafer.

FIG. 20 depicts an atomic force micrograph (tapping mode AFM) of a substrate like the one in FIG. 19(a) that has carbon nanotubes deposited in the oxidized silicon channels according to an embodiment of the methods of the present invention; (a) is a higher magnification image showing individual carbon nanotubes selectively deposited in the channels; (b) is a lower magnification image of carbon nanotubes selectively deposited in one of the channels.

FIG. 21 is an illustration of an embodiment of the present invention of a selectively deposited carbon nanotube on a substrate that is bonded to a biomolecule (protein); the carbon nanotube is shown bound to a bio-molecule (protein) through a peptide bond (—CO—NH—) with the carboxylic acid group of a surfactant adsorbed on the carbon nanotube.

FIG. 22 is a fluorescence optical micrograph of selectively deposited carbon nanotubes on an oxidized silicon substrate that are carboxylic acid grafted with a fluorescent dye.

FIG. 23 is a schematic illustration of an embodiment of a device of the present invention comprising a substrate having controllably deposited CNTs fluidically sealed to a microfluidic assembly.

FIG. 24 depicts AFM images of devices made according to embodiments of the present invention from purified HiPCO material: (a) SiO₂ surface with individual SWNTs and small bundles after deposition from solution and surfactant removal (scale bar 1 μm); (b) Cr/Au electrodes contacting SWNT material (scale bar 1 μm); (c) high resolution scan of a 4-nm diameter bundle with source and drain electrodes along top and bottom (scale bar 200 nm); and (d) three categories of I-V_{g} behavior (metallic, hybrid and semiconducting, “SC”) are observed, bias voltage is 10 mV.

FIG. 25 is an illustration of device energy bands of an embodiment of a device of the present invention; Schottky barriers form where metal leads contact a semiconducting SWNT. The Schottky barriers are asymmetric so holes conduct more readily than electrons. Carriers tunnel through the Schottky barriers, so transport is characterized by an activation energy Eₐ given by the difference between the Fermi energy E_{F} and the edge of the nearest energy band of the SWNT (here, the valence band).

FIG. 26 depicts current (I)—back gate voltage (V_{g}) characteristics for an embodiment of a device of the present invention (Device I): (a) I(V_{g}) at V_{b}=100 mV for temperatures 77-300 K; (b) thermal activation energy Eₐ as a function of V_{g}; the peak in Eₐ corresponds to Fermi energy alignment at midgap; since the maximum of Eₐ is 150 meV, the energy gap is found to be 300 meV; the lever arm α≈0.08 is inferred from the slope of a linear fit to Eₐ in the gap region; oscillations in Eₐ outside the gap region are due to single electron charging.

FIG. 27 depicts current—back gate voltage characteristics for an embodiment of a device of the present invention (Device II): (a) Temperature dependence of I(V_{g}) with V_{b}=100 mV; (b) Activation energy Eₐ as a function of gate voltage; from the maximum value of Eₐ we find that the energy gap E_{g}≧400 mV; the lever arm for this sample is α≈0.03; oscillations in I(V_{g}) and Eₐ(V_{g}) for V_{g}<−4 V are due to single electron charging effects. inset Arrhenius plot used to find the activation energy for V_{g}=−8 V.

FIG. 28 depicts the results of an embodiment of a method of the present invention for separating carbon nanotubes; average length of the distribution shown is 316 nm+/−30 nm.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable. When any variable occurs more than one time in any constituent or in any formula, its definition in each occurrence is independent of its definition at every other occurrence. Combinations of substituents and/or variables are permissible only if such combinations result in stable compounds.

As employed above and throughout the disclosure, the following terms, unless otherwise indicated, shall be understood to have the following meanings.

As used herein, the terms “nanotube” and “carbon nanotube” (“CNT”) are used interchangeably.

As used herein, the term “highly effective nanotube surfactant” refers to the class of surfactants, as exemplified by NaDDBS, which contains an aromatic group, an alkyl group having from about 4 to about 30 carbon atoms, and a charged head group. Unless indicated otherwise, use of the term “surfactant” herein refers to “highly effective nanotube surfactant”.

As used herein, the terms “single carbon nanotubes” and “individual carbon nanotubes” are synonyms that refer to freely dispersed carbon nanotubes in a dispersion that are not physically or chemically adsorbed, adhered, flocculated or aggregated to one or more other carbon nanotubes in the dispersion.

Several of the inventions described herein are based, in part, on an unexpected finding that particular surfactants having an aromatic group, an alkyl group, and a charged head group (the so-called “highly effective nanotube surfactants”), are capable of readily dispersing carbon nanotubes in aqueous media to provide colloidal carbon nanotubes. Also surprising is the discovery, as detailed herein, that a particular class of surfactants when used with an ultrasonication process is capable of providing stable dispersions of carbon nanotubes having reduced breakage of the carbon nanotubes. Another surprising discovery detailed herein is the ability to readily prepare composite materials and aligned nematic nanotube gels that contain dispersed carbon nanotubes. These novel features of the invention are advantageously used in various methods, devices, compositions and materials, as described further herein.

Generally, a colloidal particle stabilized by charged surfactants will have a so-called “double layer” where counter ions (of opposite charge to the net charge on the particle) will be in excess surrounding each dispersed particle in the continuous (typically aqueous) phase. The degree to which the counter ions are in excess will decrease with increasing distance from the dispersed particle. The thickness of this double layer will be determined by the rate at which the net charge decreases with distance from the particle which is dependent on, inter alia, the ionic strength of the colloid. The colloid will be stable as long as the ionic repulsion between these double layers keeps the dispersed particles a sufficient distance apart for short range attractive forces (such as van der Waals forces) to be insignificant. If the double layer is too thin the dispersed particles can approach sufficiently closely for these attractive forces to predominate. Thus altering the ionic strength of the colloid will effect the thickness of the double layer and hence the stability of the colloid. When the ionic strength is raised to a particular amount the double layer is so thin there is effectively no ionic repulsion between particles and the forces between the particles are purely attractive which leads to the formation of a large solid mass. Hence adding a suitable ionic salt to a colloid (often called “salting out”) will, at a certain concentration, suddenly produce an irreversible, catastrophic collapse of the dispersed particles into a distinct gelatinous clot or mass. Accordingly, the ionic strength of the aqueous media of the dispersions of the present invention are maintained at a level that maintains ionic repulsion between the carbon nanotube particles.

Without wishing to be bound by a particular theory or mechanism of operation, the present inventors postulate that the superior dispersing capability of the highly effective nanotube surfactants can be explained in terms of graphite-surfactant interactions, alkyl chain length, head group size and charge that pertain particularly to those surfactant molecules that lie along the exterior carbon nanotube surface, parallel to the nanotube central axis. It is suspected that weaker surfactants like SDS (having a dispersing capability of less than about 0.1 mg/ml) have a weaker interaction with the carbon nanotube surface compared to highly effective nanotube surfactants because they lack an aromatic group. The aromatic group is believed to permit π-like stacking of the aromatic groups onto the graphene surface of the carbon nanotubes, which significantly increases the binding and surface coverage of the surfactant molecules. The alkyl group of the class of highly effective nanotube surfactant is suspected to lie flat along the exterior surface of the carbon nanotubes, especially for carbon nanotubes having small diameters on order of the size of the alkyl groups. Thus, it is energetically favorable for the alkyl groups (e.g., alkyl chains) to lie flat along the length of the carbon nanotubes rather than bend around its perimeter (e.g., circumference). The greater the surface contacts the alkyl group has with the carbon nanotube, the greater the favorable interaction the surfactant has for the nanotube. Finally, the charged head group of highly effective nanotube surfactants permits electrostatic repulsion that leads to charge stabilization of the nanotubes via screened Coulomb interactions which, in analogy with colloidal particle stabilization, may be significant for solubilization (i.e., dispersion) in aqueous media.

The dispersions of the present invention include an aqueous medium and carbon nanotubes dispersed with at least one highly effective nanotube surfactant in the aqueous medium. Suitable surfactants have an aromatic group, an alkyl group, and a charged head group. While it is envisioned the aromatic group, the alkyl group, and the charged head group can be linked together in any chemically possible combination to provide a suitable surfactant, typically the aromatic group is disposed between the alkyl group and the head group.

As the suitable alkyl groups contain carbon atoms, the skilled person will realize that a corresponding number of hydrogen atoms will also be bonded to the carbon atoms. The alkyl group can contain alkyl branches and rings, and will preferably include at least one linear alkyl chain. The number of carbon atoms in the alkyl group will typically be from about 4 to about 30, more typically from about 6 to about 20 carbon atoms, even more typically from about 8 to about 16 carbon atoms, and most typically from about 10 to about 14 carbon atoms.

Slight chemical variations to the alkyl group, especially where the number of carbon atoms is greater than about 12, are also envisioned as within the scope of the present invention. For example, the alkyl group may contain one or several chemical groups or unsaturated covalent bonds. Examples of such a chemical variation include additional atoms besides carbon and hydrogen that are bonded to the alkyl group (e.g., nitrogen, oxygen, or sulfur) and one or more unsaturation sites bonded to the alkyl groups (e.g., alkene and alkyne groups). The addition of such chemical variations can typically be such that the adsorption of the alkyl group to the carbon nanotube is not so grossly affected so that adsorption is otherwise prevented.

While any type of aromatic group is envisioned to be suitable for the highly efficient nanotube surfactants used in the present invention, suitable aromatic groups will typically be capable of π-like stacking onto the surface of the carbon nanotubes. π-like stacking refers to the overlap of π (pi) bonds of the aromatic group of the surfactant with the π bonds of the carbon nanotubes, which provides electron delocalization. Such hydrophobic interactions typically produces an energy minimum that favors non-covalent adsorption of the surfactant on the nanotube surface. The highly effective nanotube surfactants are typically capable of non-covalently adhering to said carbon nanotubes. Many aromatic rings known in the chemical arts are suitable for use in the surfactants. Typical aromatic groups will have a carbocyclic aromatic ring, a heterocyclic aromatic ring, or any combination thereof include two or more covalently linked together. Typically, carbocyclic aromatic rings include benzenes, naphthalene, biphenylene, biphenyl, and anthracene, as well as their C₁-C₁₀ alkyl and alkene analogs known in the art, such as toluene, xylene, and vinyl benzene. A preferred carbocyclic ring is benzene. Suitable heterocyclic aromatic rings are typically carbocyclic rings having one or more carbon atoms substituted with an atom other than carbon. Typical atom substitutes in heterocyclic aromatic rings include oxygen, sulfur, and nitrogen. The conditions of aromaticity can be met by many nitrogen-, oxygen-, and sulfur-containing ring groups. Heterocyclic aromatic ring groups have chemical properties similar to those of benzene and its derivatives. Examples of suitable heterocyclic aromatic ring groups include pyridine, purine, pyrimidine, pyrazine, pyridazine, pyrrole, imidazole, 1,3,4-triazole, tetrazole, furan, indole, oxazole, isoxazole, thiophene, thiazole, 1,2,3-thiadiazole, 1,2,4-thiadiazole, 1,3,5-trizene, quinoline, isoquinolene, acridine, and any combination thereof.

While any type of charged head group is envisioned to be suitable for the highly efficient nanotube surfactants used in the present invention, suitable charged groups will typically be capable of carrying a positive or negative charge in aqueous media. Suitable charged head groups also capable of being electrostatically shielded from each other in aqueous media to affect dispersion. Accordingly, suitable charged head groups include any cationic, anionic, or amphoteric group that is known to be useful in preparing surfactants and dispersants for use in preparing aqueous particles dispersions. Examples of suitable anionic groups include sulfate groups and carboxylic, sulfonic, phosphoric and phosphonic acid groups which may be present as free acid or as water-soluble ammonium or alkali metal salts. Typically, the alkali metal salt will have a counterion selected from the Group IA elements, such as sodium, and potassium salts, e.g. sodium carboxylates and sulfonates, or any combination thereof.

Combinations of anionic groups are also possible. Surfactants having an anionic charged head group may further contain one or more cationic groups as long as it has an overall anionic charge. If the surfactant is to have predominantly a cationic charged head group, then the reverse is true. Examples of suitable cationic head groups include sulfonium groups, phosphonium groups, acid addition salts of primary, secondary and tertiary amines or amino groups and quaternary ammonium groups, for example where the nitrogen has been quaternized with methyl chloride, dimethyl sulfate or benzyl chloride, typically acid addition salts of amines/amino groups and quaternary ammonium groups.

The highly efficient nanotube surfactants are derived from synthetic and natural sources and preferably are water-soluble or water-dispersible. Many suitable surfactants are commercially available from various companies, such as The Akzo Nobel Company in The Netherlands (http://www.se.akzonobel.com/misc/ProductOverviewSurfactantsEurope.pdf). In a preferred embodiment, the surfactant includes anionic surfactants like alkylaryl sulfates and alkylaryl ethersulfates, alkylaryl carboxylates, alkylaryl sulfonates, alkylaryl phosphates and alkylaryl etherphosphates. Typical anionic surfactants includes, sodium butylbenzene sulfonate, sodium hexylbenzene sulfonate, sodium octylbenzene sulfonate, sodium dodecylbenzene sulfonate, sodium hexadecylbenzene sulfonate, and preferably sodium dodecylbenzenesulfonate, and combinations thereof. Suitable surfactants preferably include an alkaline salt of a Cₙ alkyl benzene sulfonate, where n is between about 8 and about 16.

Examples of suitable surfactants having a cationic head group include cocobenzyldimethylammonium chloride, coco(fractionated)benzyldimethylammonium chloride, di(hydrogenated tallow)benzylmethylammonium chloride, and (hydrogenated tallow)benzyldimethylammonium chloride. Suitable cationic surfactants include those containing at least one quaternary ammonium compounds. Further suitable cationic surfactants include quaternary di- and polyammonium compounds.

In certain embodiments of the present invention, the surfactant contains a plurality of alkyl groups that are bonded to the aromatic group, an example being two alkyl chains attached. Typically, however, the surfactant will have a single alkyl chain.

In other embodiments of the present invention, the surfactant may further contain one or more hydrophilic chains. The one or more hydrophilic chains may be disposed on the surfactant in any combination, for example a hydrophilic chain may be connected to the charged head group, the aromatic group, or the alkyl group. A hydrophilic chain may also be disposed between any two of the charged head group, the aromatic group, or the alkyl group, e.g., a hydrophilic chain could separate the charged head group from the aromatic group. In these embodiments, the hydrophilic chains could function as a spacer. Suitable hydrophilic chains include polymers of alkyloxide monomers, such as ethyleneoxide and propyleneoxide, wherein the degree of polymerization is at least two.

Any type of carbon nanotube can be dispersed using the methods and surfactants as described herein. Although not an exhaustive listing of all known types of carbon nanotubes that can be used, a number of suitable carbon nanotubes that can be used in various embodiments of the present invention include the following: single-wall carbon nanotubes, multi-wall carbon nanotubes, armchair carbon nanotubes, zigzag carbon nanotubes, chiral carbon nanotubes, carbon nanofibers, carbon nanotoroids, branched nanotubes (e.g., as disclosed in U.S. Pat. No. 6,322,713, the details pertaining to the preparation branched nanotubes is incorporated by reference herein), carbon nanotube “knees”, coiled carbon nanotubes (L. P. Biro et al., Mat. Sci. and Eng. C 19 (2002) 3-7), or any combination thereof. Many types of carbon nanotubes are commercially available. Several procedures known in the art are capable of synthesizing a variety of carbon nanotubes. For example, multi-wall carbon nanotubes can be made by the arc method known in the art and SNWTs can be made by the high-pressure carbon monoxide (“HiPCO”) method known in the art and supplied commercially by Carbon Nanotechnologies, Inc. (Houston, Tex.). SNWTs can be synthesized by the laser-oven method and supplied commercially by Tubes@Rice (Rice University, Houston, Tex.). Carbon nanotoroids can be made by the HiPCO and laser-oven methods. Branched nanotubes can be made according to U.S. Pat. No. 6,322,713, the details pertaining to the preparation of branched nanotubes is incorporated by reference herein. Carbon nanofibers are commercially available from Electrovac GesembH, Klosterneuberg, Austria. Carbon nanofibers typically are hundreds of micrometers long having diameters from about 70 to about 500 nm, having greater than about 100 square meters per gram (m²/g) active chemical surface area. Chemical vapor deposition (CVD) methods are also capable of synthesizing carbon nanotubes.

While the carbon nanotubes that are useful in the present invention have mostly carbon atoms, it is envisioned that at least a portion of the carbon atoms may be substituted with any of a variety of non-carbon atoms. Likewise, while chemical modification of the carbon nanotubes is not typically required for practicing the present invention as described herein, nevertheless, the carbon nanotubes may be chemically modified. In this regard, chemical modifications may include functionalization with any of a variety of chemical functional groups and molecules as known and practiced in the nanotube art.

The highly effective nanotube surfactants enable the preparation of aqueous dispersions having very high concentrations of dispersed carbon nanotubes. Although any concentration of carbon nanotubes in the dispersion is possible, generally the nanotube concentration will be less than about 500 mg/ml, more typically less than about 200 mg/ml, even more typically less than about 100 mg/ml, even further typically less than about 50 mg/ml, and most typically less than about 25 mg/ml. Although very small concentrations of carbon nanotubes can be prepared according to the present invention, for example less than about 0.001 mg/ml, the nanotube concentration is typically at least about 0.001 mg/ml, more typically at least about 0.01 mg/ml, even more typically at least about 0.1 mg/ml, and even further typically at least about 0.5 mg/ml. Accordingly, the nanotube concentrations can be varied over a wide range for a variety of applications.

In certain embodiments of the present invention, the carbon nanotube dispersions will have a high number percentage of individual carbon nanotubes. In these embodiments, the number percentage of single carbon nanotubes is typically at least about 50 number percent based on the total number of carbon nanotubes longer than 50 nm. This counting “cut-off” of 50 nm is conveniently selected based upon analytical procedures for measuring the length distribution of carbon nanotubes as described hereinbelow, e.g., using atomic force microscopy (AFM) coupled with computer software techniques for counting individual nanotubes. In other embodiments the number percentage of single single-wall carbon nanotubes is typically at least about 75 percent, and in other embodiments this percentage is at least about 90 percent. The present invention is not limited to the use of such a counting cutoff, as it will be readily apparent to those skilled in the art in view of the present disclosure that other counting methodologies and analytical instrumentation may be conveniently selected.

In certain embodiments it is desirable that the mean length of a plurality of carbon nanotubes is typically at least about 120 nm. In embodiments where longer carbon nanotubes are desired, the mean length of the carbon is at least about 300 nm, and even at least as high as about 500 nm. When single carbon nanotubes are desired, the number percentage of single carbon nanotubes greater than 50 nm in length in the dispersions will typically be at least about 50 percent. As used herein, the term “mean length” typically refers to the mean end-to-end distance along the axis of cylindrical-shaped carbon nanotubes. For carbon nanotoroids, the term “mean length” refers to the mean of a the outside diameters of a plurality of toroids, i.e., the mean of the diameters of the outer circles. For branched carbon nanotubes, the term “mean length” refers to the mean of the longest distance from one branch end to another branch end. Other measures of length for various forms of nanotubes will be apparent from their respective forms.

While any type of carbon nanotube can be dispersed according to the methods as provided herein, in a preferred embodiment of the present invention the carbon nanotubes are single-walled carbon nanotubes (abbreviated herein as “SWNT”). While the SWNTs are readily dispersed as aggregates of two or more SWNTs using the surfactants and methods described herein, it is typical that a portion of the SWNTs will be dispersed as single SWNTs. When single SWNTs are present in the various inventions as described herein, in certain embodiments it is desirable that the mean length of the collection of single SWNTs is typically at least about 120 nm. In embodiments where longer single SWNTs are desired, the mean length of the single SWNTs can be at least about 300 nm, and even at least as high as about 500 nm. When single SWNTs are desired, the number percentage of single SWNTs greater than 50 nm in length in the dispersions will typically be at least about 50 percent.

In certain embodiments of the present invention stable dispersions of carbon nanotubes typically include a surfactant to disperse and stabilize the nanotube particles. The amount of surfactant needed will vary depending on the surfactant's composition, the aqueous media, the chemical nature of the carbon nanotubes, and the total surface area of the carbon nanotubes that are to be dispersed. In various embodiments the present invention, the weight ratio of carbon nanotubes to surfactant is typically in the range of from about 5:1 to about 1:10. More surfactant is typically needed to increase the stability of the dispersions. The term “stability” used herein refers to the ability of the dispersed nanotubes to remain dispersed in solution without aggregation or flocculation. A high degree of stability is typically evidenced by a dispersion with little or no flocculation or aggregation developing upon standing for more than two weeks in a sealed vessel at ambient conditions. High degrees of stability are commonly achieved according to the methods of the present invention when the weight ratio of nanotubes to surfactant is in the range of about 1:5 to about 1:10. High degrees of stability are important for use in products in which liquid dispersions commonly stand for at least a week prior to their use (e.g., electronic chemicals processing of liquid photoresists). Lower degrees of stability can be achieved with lower relative amounts of surfactant. For example, a weight ratio of nanotubes to surfactant of about 3:1 can be used for keeping SWNTs dispersed for about a week in water. Thus, applications in which carbon nanotube dispersions are used in less than a weeks' time after preparation require even less surfactant. Because excessive amounts of surfactant can deleteriously alter various other properties in their applied use, it is typical to use just enough surfactant that permits dispersion and stability of the carbon nanotubes. Dispersions that are not stable (i.e., those in which the nanotubes begin to flocculate or aggregate upon standing at ambient conditions) are typically evidenced by at least one of the following: an increase in viscosity; an increase light scattering; formation of a liquid phase separation containing a nanotube-rich phase and a nanotube-poor phase; and formation of a solid clot or gel phase.

In various embodiments of the present invention the carbon nanotubes can be stabilized using steric hindrance, charge stabilization, or both steric hindrance and charge stabilization to prevent the flocculation and aggregation of dispersed nanotubes. The carbon nanotubes are typically charge stabilized using one of the suitable surfactants described herein. Without being bound by a particular theory, any of the suitable surfactants apparently disperse the carbon nanotubes through the operation of a portion of the alkyl group and aromatic group being adsorbed to the carbon nanotubes under the influence of dispersive forces, and by operation of the charged head group being situated in the aqueous solution to form a charge shield surrounding the carbon nanotube. Charge shielding of a plurality of nanotubes in the aqueous medium gives rise to a stable dispersion. Although many of the suitable surfactants described herein have a single alkyl group, certain embodiments the surfactants may have two or more alkyl groups. Likewise, the surfactant typically needs just one aromatic group, however two or more aromatic groups can be used. In a similar fashion, the surfactants can have more than one charged head group, although a single head group is typically required. Surfactants having any combination of two or more alkyl groups, two or more aromatic rings, or two or more charged head groups are also envisioned as useful for preparing the dispersions as described herein.

The dispersions of the present invention include an aqueous liquid medium. As used herein, the term “aqueous medium” means including water. As used herein, the term aqueous liquid phase refers to the portion of the dispersion not including the surfactant and carbon nanotubes. While any amount of water in the aqueous medium can be used, the amount of water contained by the aqueous liquid phase is typically at least about 50 weight percent water, more typically at least about 70 weight percent water, even more typically at least 85 weight percent water, further typically at least about 90 weight percent water, and most typically at least about 95 weight percent, and in certain embodiments up to 100 weight percent water. While a majority of the aqueous medium will typically be water, it may also contain up to one or more solvents or solutes different than water. Typically, the aqueous liquid phase will include up to about 50 weight percent of a solvent different than water. This percentage is more typically up to about 30 weight percent, even more typically up to about 15 weight percent, further typically up to 1 about 0 weight percent, and most typically up to about 5 weight percent of a solvent different than water. In certain embodiments no other solvents are present other than water in the aqueous liquid phase.

Preparation of the dispersions of carbon nanotubes can be carried out using a variety of known particle dispersion methodologies, including but not limited to the use of high-shear mixers (e.g., homogenizers), media mills (e.g., ball mills and sand mills), and sonicators (e.g., ultrasonicators, megasonicators). In a more typical method of dispersing carbon nanotubes, there is provided a method that includes mixing an aqueous medium, carbon nanotubes, and surfactant in a low-power, high-frequency bath sonicator. In carrying out this method, the mixing time is selected in the low-power, high frequency bath sonicator so that the carbon nanotubes become sufficiently separated from each other, contacted with the surfactant, and stabilized in the aqueous medium such that the carbon nanotubes remain substantially suspended in the aqueous phase upon cessation of input of the sonicator energy into the dispersion. Greater mixing times typically lead to greater degrees of dispersion of the carbon nanotubes. A suitable mixing time in a bath sonicator to achieve some level of dispersion of carbon nanotubes is typically in the range of about several minutes to about tens of hours, and is more typically at least about two hours, even more typically at least about four hours, even more typically at least about eight hours, and most typically in the range of from about 16 to about 24 hours. As used herein, the term “some level of dispersion” means that there has been a measurable diminution in aggregate size of carbon nanotubes, e.g., the preparation of single SNWTs from undispersed SNWT powder containing aggregates.

In carrying out this method, suitable bath sonicators typically have a power in the range of from about five watts to about 75 watts. Likewise, suitable bath sonicators have an operating frequency in the range of from about 20 kilohertz (“kHz”) to about 75 kHz.

The methods of preparing the dispersions of the present invention can be carried out with any one or a combination of surfactants as described herein. The present methods can also be carried out wherein a minor portion of the surfactants used to be other surfactants known in the art, e.g., those not containing at least one of an alkyl group, and aromatic group, or a charged head group. In one embodiment of carrying out the present method, more than half of the surfactant based on weight will include an alkaline salt of a Cₙ alkyl benzene sulfonate, where n is between about 8 and about 16.

In certain embodiments of the present invention, the mixing time is typically selected to give rise to at least about 50 number percent of the dispersed carbon nanotubes being single SWNTs. In these embodiments, it is also typical that the mixing time is selected to give rise to the mean length of single SWNTs being at least about 300 nm, and more typically at least about 500 nm. In carrying out these embodiments, the concentration of the surfactant based on the total volume of the dispersion is typically less than the critical micelle concentration (CMC) of the surfactant in the aqueous medium. Even more typically, the amount of free surfactant in the aqueous medium portion of the dispersion is less than the CMC of the surfactant based on the total volume of the aqueous medium. As used herein, the critical micelle concentration is the concentration at which micelles of surfactant form upon addition of surfactant to the aqueous medium. The critical micelle concentration typically varies with the composition of the surfactant, the composition of the aqueous medium, and the temperature of the aqueous medium.

In certain embodiments of the present invention, applications of the carbon nanotube dispersions require that the electronic properties of the dispersed carbon nanotubes are essentially the same as the electronic properties of the carbon nanotubes prior to mixing. This can be carried out using carbon nanotubes that are not chemically modified, such as unmodified SWNTs. Thus, in one embodiment of the present invention there are provided methods of preparing SNWT dispersions from unmodified SNWTs using the methods of the present invention.

In additional embodiments of carrying out the methods of the present invention, the dispersed carbon nanotubes can be further processed using one or more processing steps for to carryout any of a number of post-dispersion processing steps. One post-dispersion processing step is classifying the nanotubes by size. A related post-dispersion processing step includes one or more separation steps to separate the carbon nanotubes according to length, shape, diameter, type, or any combination thereof. In carrying out these methods any one or more of known methods capable of classifying particles or soluble macromolecules can be used. For example, in one embodiment of the present method, there is provided a further step of electrophoretically separating the dispersed carbon nanotubes. Although the additional separation step typically occurs after the nanoparticles are prepared, it is possible that separation may also occur prior to dispersion, during dispersion, or both prior to and during dispersion.

The aqueous medium of the dispersions described herein can be partially or fully removed from the dispersion. In another embodiment there is provided a composition having carbon nanotubes and surfactant comprising an alkyl group having from about 4 to about 30 carbon atoms, an aromatic group, and a charged head group. Typically, such compositions will have at least a portion of the surfactant adsorbed to the exterior surface of the carbon nanotubes. This is especially useful for preparing nanotube compositions in the form of a powder, film, pellets, or any combination thereof. Powder, particle and pellet forms of the composition can be advantageously used as additives in various materials, including paints, coatings, adhesives, plastics, composites, and various engineering materials. Additional binder materials can be added to hold powdery compositions as pellets or films. The compositions of nanotubes and surfactant may also be mixed with a non-aqueous liquid, such as an organic solvent, electrolyte, or oil to prepare oil-based carbon nanotube dispersions.

The composite materials of the present invention suitably include a solid matrix, carbon nanotubes and surfactant dispersed within the solid matrix. Suitable solid matrix materials include a polymeric material, a ceramic material, a metal oxide material, a metallic material, a semiconducting material, a superconducting material, an insulating silicon-containing material, and any combination thereof. Suitable polymeric materials include a linear polymer, a branched polymer, a crosslinked polymer, a grafted polymer, a block co-polymer, a ceramic precursor, or any combination thereof. Typically the solid matrix material includes a curable polymer resin precursor that can be hardened upon subjecting the resin to light, heat, radiation, or time for ambient curing. Suitable ceramic materials include any of a variety of ceramic materials that are suitably derived using sol-gel techniques. Examples of such ceramic materials include silicon dioxides, titanium dioxides and aluminum oxides. Typical sol-gel precursors that can be mixed with the carbon nanotube dispersions and compositions of the present invention include silicates for the preparation of silica gels, as well as a variety of silanes, silicones, germanes, alkoxides, tin compounds, lead compounds, metal organic compounds, for preparing any of a variety of known sol-gel solid matrix materials. Many sol-gel precursors are commercially available from a variety of suppliers, such as Gelest, Inc., Morrisville, Pa., and The E. I. DuPont Company, Wilmington, Del.

Composites of the present invention can have a variety of forms, and can take the form of a pellet, powder, or film. Such composite materials can be further processed into a variety of engineering materials and coatings.

Methods of preparing the composites typically include dispersing carbon nanotubes and surfactant in a hardenable matrix precursor, the surfactant including an alkyl group having from about 4 to about 30 carbon atoms, an aromatic group, and a head group; and hardening the precursor. In these methods, suitable hardening of the precursor typically includes curing the precursor with at least one of light, heat, radiation and time. Typical hardenable matrix precursors having these capabilities include any of the well-known cross-linkable organic-based multifunctional monomeric and oligomeric precursors, such as epoxies, polyesters, and ethylenically unsaturated styrenics. Sol-gel precursors are also useful as the hardenable matrix precursor for preparing ceramic metal oxide matrices.

In another embodiment of the composite materials of the present invention, the hardenable matrix precursor is a polymer capable of solidifying upon cooling to a temperature being lower than its glass transition temperature, its crystalline melt transition, its order-disorder transition temperature, or any combination thereof. A myriad of polymers having such properties are well-know in the art and can be used in the present invention. Examples of suitable polymers include but are not limited to polyolefins, polycarbonates, polyacrylics, polymethacrylics, polystyreneics, polyetherimides, polyamides, polyacrylamides, polyaklylacrylamides, polyimides, polyalkylimides, as well as random copolymers, block copolymers and blends thereof.

Assemblies having a substrate, and carbon nanotubes and surfactant adjacent to the substrate can also be fabricated according to the present invention. By use of the phrase “adjacent to the substrate” is meant that the carbon nanotubes and surfactant are limited in their physical location to an area in contact with, or in proximity to, the substrate surface. The assemblies are designed so that carbon nanotubes become arranged upon the surface of the substrate as they come in contact with the surface. Typically, the carbon nanotubes and the surfactant will be in the form of a dispersion in the presence of a solvent, such as an aqueous medium. Although this aspect of the present invention is typically carried out with an aqueous medium, it is not necessary for such a medium to be present. For example, the carbon nanotubes can assemble on the surface of a substrate using a suitable liquid-less mass transport system. A suitable liquid-less mass transport systems includes chemical vapor deposition processes.

In preparing the assemblies of the present invention the carbon nanotubes typically self-assembled on the substrate. The term “self-assembly” as used herein means that the carbon nanotubes arrange themselves in a fashion that is directed by their chemical, physical, and chemical-physical interactions between each other. Examples of self-assembly of carbon nanotubes includes alignment of the central axes of a plurality of carbon nanotubes in generally the same direction, herein referred to as “nematically-aligned”. In assisting the orientation of the carbon nanotubes in a particular direction, the surfactant is typically adsorbed to the exterior surface of the carbon nanotubes, which permits molecular mobility and orientation of the carbon nanotubes in a particular direction under the influence of an orienting field. In the case of carbon nanotubes that self-assemble on surfaces from solution, and without being bound by a particular theory of operation, it is believed that the substrate surface imposes an boundary-directed confinement of normal molecular motion (i.e., Brownian motion), thereby giving rise to the assembly of carbon nanotubes in a particular orientation at the substrate surface.

Assembling carbon nanotubes from one or more of the dispersions provided by the present invention on a surface of a substrate can be carried out by contacting a dispersion containing an aqueous medium, carbon nanotubes and surfactant to a substrate. The combination of carbon nanotubes and surfactant present in the dispersions of the present invention generally are capable of preferentially adsorbing on a variety of substrate surfaces. Preferential adsorption is generally driven by favorable surface energy thermodynamics that drive surfactant and carbon nanotubes from the dispersion out of solution and onto a surface. Carbon nanotubes can preferentially adsorb to the surface in an end-to-surface orientation that gives rise to self-assembly. Self-assembly will depend inter alia on a variety of parameters, including the self-organizing dispersive forces, the nature of the surfactant, the type and composition of the carbon nanotubes, the nature of the surface, and the quality of the dispersion. Self-assembled carbon nanotubes standing end-to-surface are capable of tightly packing close to the substrate surface, which typically reduces the overall enthalpy of the assembled system.

Balancing the enthalpic components of the energetics of the system is its entropy. Entropy will typically drive the assembled system to disorganization. Because entropic components of the energy of a system decreases as the number of molecules in a system increases, a dispersion of longer carbon nanotubes will have a greater tendency to self-assemble on a substrate surface compared to a dispersion of shorter carbon nanotubes. In comparison to earlier methods, the methods of the present invention for preparing high weight fraction dispersions of relatively longer carbon nanotubes further enables the preparation of self-assembled carbon nanotube assemblies on substrate surfaces.

One use of the carbon nanotubes of the present invention is to provide solid media that can be used in detecting chemical and biological substances. In this use, the solid media includes a substrate for receiving chemical compounds, biological material, or both biological material and chemical compounds for detection. Here, the substrate typically includes carbon nanotubes and surfactant adsorbed thereon, the surfactant comprising an alkyl group having between about 6 and about 30 carbon atoms, an aromatic group, and a charged head group. In one embodiment, the solid media are prepared by adsorbing surfactant to the exterior surface of the carbon nanotubes, the carbon nanotubes and surfactant adsorbed to the substrate. Typical substrates for solid media for detecting a variety of substances include both organic and inorganic porous materials, such as polymeric materials, ceramic materials, zeolites and ion-exchange resins. In certain embodiments of the solid media of the present invention, it will be advantageous for the carbon nanotubes to be self-assembled on the substrate. In this embodiment when the carbon nanotubes are pointing their ends away from the surface, their ends are readily capable of attaching chemical and biological substances for analysis.

In a related embodiment, the solid media includes carbon nanotubes that are capable of adsorbing protons to give rise to a detectable signal. In this embodiment, the carbon nanotubes contain openings that are capable of receiving atomic, molecular, or both atomic and molecular species within their interior spaces.

In a related embodiment, the solid media includes carbon nanotubes that are chemically functionalized to adsorb specific biological material or chemical compounds to give rise to a detectable signal. A variety of chemical functionalization schemes are known in the separations literature, a number of which are capable of modifying the surfaces of carbon nanotubes. Specific examples include the addition of nucleic acids that hybridize with genetic material, acidic moieties that bind basic moieties of chemical compounds, basic moieties that bind acidic moieties of chemical compounds, proteomic and enzymatic fragments for binding proteins, and antigens for binding viruses.

Composites of aligned carbon nanotubes, especially containing single wall carbon nanotubes (SWNTs), are among the most sought after materials in nanotube science and technology. The present inventions are capable of providing such composite materials, especially those containing large domains of oriented SWNTs referred to herein as nematic nanotube gels. These composite materials are enabled by use of the highly efficient nanotube surfactants as described above.

The methods of preparing carbon nanotube gels according to the present invention typically include the steps of providing a dispersion of carbon nanotubes, solvent, gel precursor, and surfactant, gelling at least a portion of the gel precursor to form a gel, and subjecting the dispersion, the gel, or both the dispersion and the gel to an orienting field to give rise to a nematic orientation of the carbon nanotubes. By nematic orientation is meant that, on average, the carbon nanotubes are aligned in a particular direction. When aligned in a particular direction, the carbon nanotubes will typically have a finite order parameter greater than the fluctuation-induced order parameter at the order-disorder transition.

In preparing the nematic nanotube gels, the concentration of the carbon nanotubes in the dispersion of carbon nanotubes, solvent, gel precursor, and surfactant, is sufficiently low so that carbon nanotubes remain substantially disordered in the dispersion. By substantially disordered is meant that a majority of the carbon nanotubes is capable of being oriented in any direction through action of Brownian motion. As the length of the carbon nanotubes increases, the concentration needed to achieve a substantially disordered dispersion typically decreases. Typically, this concentration is less than about 20 mg/ml, more typically less than about 10 mg/ml, and even more typically less than about 5 mg/ml, further typically less than about 2 mg/ml, and even further typically less than about 1 mg/ml, the concentration being based on the total weight of the carbon nanotubes, solvent, surfactant, and gel precursor. Likewise, the concentration of carbon nanotubes in the dispersions for preparing nematic nanotube gels will typically be at least about 0.001 mg/ml, more typically at least about 0.01 mg/ml, even more typically at least about 0.1 mg/ml, and further typically at least about 0.5 mg/ml, the concentration being based on the total weight of the carbon nanotubes, solvent, surfactant, and gel precursor.

In several embodiments of the present invention the nematic nanotube gels may contain SWNTs having a particular degree of single dispersed nanotubes, a particular mean length, or both a particular degree of single dispersed nanotubes and a particular mean length. In these embodiments, the number percentage of single SWNTs is typically at least about 50 percent, more typically at least about 75 percent, and even more typically at least about 90 percent. In these embodiments, the mean length of single SWNTs is typically at least about 120 nm, more typically at least about 300 nm.

In preparing the dispersions of carbon nanotubes, solvent, surfactant, are typically first mixed to provide a weight ratio of carbon nanotubes to surfactant in the range of from about 5:1 to about 1:10. Typically the gel precursor is soluble in the solvent used, the solvent typically being an aqueous medium as described above. The addition of gel precursor to a dispersion of carbon nanotubes is typically carried out in a fashion so that the carbon nanotubes remain charge stabilized in the dispersion. This can be carried out using any one of, or a combination of, a variety methods know in the art of preparing composite materials containing particle dispersions. For example, in one embodiment, a gel precursor which is soluble in the solvent can be slowly added to a carbon nanotube dispersion while agitating or sonicating the dispersion. In another embodiment, the aqueous media can be removed to form a powdery material, which is simultaneously or subsequently dispersed into a gel precursor.

Suitable gel precursors used in the present invention can be any of a variety of monomer, oligomer, polymer, sol-gel ceramic precursor, or any combination thereof. Many types of materials are known to those skilled in the art of composites and are commercially available. Suitable polymer gel precursors will typically be soluble at their use concentration in the dispersion of carbon nanotubes, solvent, surfactant, and gel precursor prior to hardening. Typically, for the purposes of hardening the composite materials, the gel precursor will contain a monomer that is polymerizable via chain growth, step-growth, or any combination of chain-growth and step-growth polymerization mechanisms. Suitable monomers capable of chain-growth polymerization mechanisms contain at least one ethylenically-unsaturated chemical group. Examples of ethylenically unsaturated monomers include acrylic monomers, alkylacrylic monomers, acrylamide monomers, alkylacrylamide monomers, vinyl acetate monomers, vinyl halide monomers, diene monomers, styrenic monomers, or any combination thereof. Examples or ceramic gel-precursors suitable in this embodiment of the present invention are indicated above.

In various embodiments using a polymer gel precursor, a crosslinker may also be included. Crosslinkers typically have two or more functional groups capable of covalently bonding to two or more polymer chains, such as any of the many multi-ethylenically unsaturated monomers that are well known in the polymerization art. The polymer gel precursor may further include an initiator, such as a free-radical initiator that is suitable for the initiation of chain polymerization of ethylenically unsaturated monomers. Various free-radical initiators are commercially available. Various suitable free-radical initiators are thermally-activated as well as activated by light such as UV radiation. The polymer gel may further include an accelerator. Accelerators are typically used to reduce the activation energy required by any of the initiation, polymerization and crosslinking (e.g., curing) processes. Many accelerators are well-known in the polymerization art, such as the teaching of the use of organophosphorus compounds for accelerating the curing of epoxy resin compositions, in U.S. Pat. No. 6,512,031, the portion of which pertaining to the curing of epoxy resins is incorporated herein by reference thereto.

Suitable orienting fields that can be used to nematically align the carbon nanotubes include pressure fields, magnetic fields, thermodynamic fields, electric fields, electromagnetic fields, shear fields, gravitational fields, as well as any combination thereof. Suitable thermodynamic fields include any type of thermodynamic perturbation on the dispersion that gives rise to a volumetric phase transition. Examples of thermodynamic perturbations include a change in temperature, a change in composition, a change in pressure, and any combination thereof.

In one embodiment, a thermodynamic field is used to nematically align carbon nanotubes by changing the temperature of the gelled carbon nanotube dispersion to give rise to a volumetric phase transition. Here, the volumetric phase transition gives rise to a decrease in volume of the solvent-gel system, thus resulting in a volume-compression transition. In this embodiment, the carbon nanotubes are typically first dispersed at low volume fraction in a gel having zero or a very low degree of order. A volume-compression transition of the gel is typically applied to induce the randomly-dispersed carbon nanotubes to become aligned, which gives rise to a greater degree of order in the system. Hallmark liquid crystalline defects in these materials are typically observed, as well as a novel buckling of the walls accompanying defect formation arising from the disorder (i.e., isotropic) to order (i.e., nematic) transition. This transition from an isotropic to a nematic phase is typically concentration-dependent, which can be quantitatively measured by analysis of the tube order parameter.

Volumetric phase transitions used in the present invention typically arise from a change in temperature. While the change in temperature may arise from a lowering of the temperature, the volumetric phase transition typically arises from an increase in temperature. Generally, the volumetric phase transition arises from an incompatibility between the gel and the solvent. For example, this incompatibility between the gel and the solvent typically results from a decrease in a specific attractive interaction. An example of specific attractive interactions that can decrease upon the increase of temperature is hydrogen bonding. In one embodiment, when the gel is a polymer gel comprising a network, and the volumetric phase transition arises upon increasing temperature, the polymer network effectively becomes hydrophobic and solvent is expelled from the gel. The expelling of solvent from the gel reduces the overall mass of the system. In view of the fact that density typically remains invariant, a decrease in volume occurs that results in a volume-compression transition.

As will be appreciated by those skilled in the polymer gel art, the properties of polymer gels depends on a variety of parameters, including the nature and composition of the solvent. Because controlling hydrophilic-hydrophobic interactions with temperature relies upon the existence of hydrogen bonding interactions, in one embodiment of the present invention the solvent typically includes at least about 50 weight percent water.

After subjecting the dispersion to an orienting field to provide a thermodynamic phase transition that gives rise to two or more phases, the phases may be separated. An example of such separation is carried out in embodiments wherein a solvent-rich phase is removed that is expelled from the gel during or after subjecting the gel to a volumetric phase transition.

In embodiments in which the gel undergoes a volumetric phase transition, the ratio of the volume of the gel before the volumetric phase transition to the volume of the gel after the volumetric phase transition is typically in the range of from about 1.1:1 to about 30:1, and more typically in the range of from about 4:1 to about 16:1.

One of the properties of the nematic nanotube gels is that they will typically exhibit birefringence subsequent to subjecting them to the orienting field. Birefringence pertains to the nematic nanotube gel having an anisotropic refractive index, e.g., the refractive index of the nematic nanotube gel in the direction along the nanotube axes is different than the refractive index across the nanotube axes.

In addition to subjecting the dispersions containing nanotubes, solvent, surfactant, and gel precursors to thermodynamic phase transitions that gives rise to nematically oriented carbon nanotubes, the dispersions can also be subjected to other thermodynamic phase transitions in various embodiments of the present invention. In one such embodiment, the method can further include the step of micro-phase separating at least one component of the dispersion into nanotube rich/gel poor and nanotube poor/gel rich phases. In this embodiment of the present invention, the gel can be a polymer gel, and the micro-phase separating step can be carried out under conditions giving rise to polymerization-induced phase separation.

In another embodiment of the present invention, the orienting field is a pressure field giving rise to transport of at least a portion of the solvent out of the gel. In this embodiment, the gelled material containing the carbon nanotubes is typically confined to a restricted geometry vessel. Suitable restricted geometry vessels include capillary tubes, microchannels, nanochannels, and substrate surfaces. Substrates surface embodiments typically have thin films of gelled material being situated thereon. In this embodiment, the gel is typically confined to a restricted geometry vessel during transport of at least a portion of the solvent out of the gel. Typically, the gel remains confined to the restricted geometry vessel after transport of at least a portion of the solvent out of the gel. In additional embodiments the gel may be confined to a restricted geometry vessel both during and after transport of at least a portion of the solvent out of the gel.

In carrying out the embodiments of the present method wherein the orienting field is a pressure field, a suitable pressure field is the application of a pressure to the gel that is lower than the partial pressure of the solvent in the vapor phase. In this embodiment, one typically applies vacuum to at least one open end of the restricted capillary vessel. In this case solvent molecules entering the vapor phase are carried away towards the vacuum source, which gives rise to a decrease in the solvent concentration in the gel. The decreasing concentration in solvent results in an increase in carbon nanotube concentration. As the carbon nanotubes become more crowded, they align thus forming a nematic nanotube gel.

In another embodiment of the method of the present invention the orienting field is a magnetic field for magnetically inducing alignment of carbon nanotubes in gel material. In this embodiment, carbon nanotubes are typically aligned inside gel materials by applying a magnetic field to the dispersion while the gel precursor is gelling. Typically the dispersion is confined to restricted geometry vessel, but such a vessel is not essential. Any type of magnetic field source can be used as long as the magnetic field strength is typically at least about 0.01 Tesla (T), more typically at least about 0.1 T, and even more typically at least about 1 T. A suitable magnetic field source is a strong permanent magnet, and more typically a superconducting magnet is used. The strongest magnets are permanent, superconducting, and pulsed magnets. Permanent magnets retain their magnetism for a long time. The neodymium-iron-boron magnet is a strong permanent magnet that can produce a field of about 0.1 T. Carbon nanotubes containing iron (e.g., as an impurity) readily align in a magnetic field of about 9 T. When the carbon nanotubes are substantially free of iron, a magnet field of about 20 T is typically required for alignment. Superconducting magnets are a type of electromagnet that produces a magnetic field from the flow of electric current through a material having essentially zero electrical resistance. A superconducting magnet can reach field strengths as high as about 13.5 T, and typical superconducting magnets that are readily used in this embodiment of the present invention have magnetic field strengths typically in the range of from about 1 T to about 9 T. A pulsed magnet provides brief, but extreme magnetic fields as high as about 60 T. The limit to the upper magnetic field strength is typically limited by the type of magnet that is used, which is typically less than about 60 T.

The strength and duration of a suitable magnetic field that is required for orienting the carbon nanotubes in the gel will typically depend on the gel viscosity and the average length and concentration of the carbon nanotubes. In many applications the viscosity of the gel while the dispersion is being subjected to the magnetic field is typically in the range of from about 1 centipoise to about 5000 centipoise. Likewise, the concentration of carbon nanotubes in the dispersions containing gel precursor, nanotubes, surfactant and solvent is typically in the range of from about 0.01 mg/ml to about 500 mg/ml based on the total dispersion, and is more typically at least about 0.1 mg/ml, even more typically at least about 0.5 mg/ml, and typically less than about 200 mg/ml, more typically less than about 100 mg/ml, and even more typically less than about 30 mg/ml.

In certain embodiments of the method of the present invention wherein a magnetic orienting field is used, at least a portion of the carbon nanotubes align end-on-end giving rise to carbon nanotube needles. In this embodiment, the method may further include the step of removing solvent from the gel to provide carbon nanotube needle composite materials.

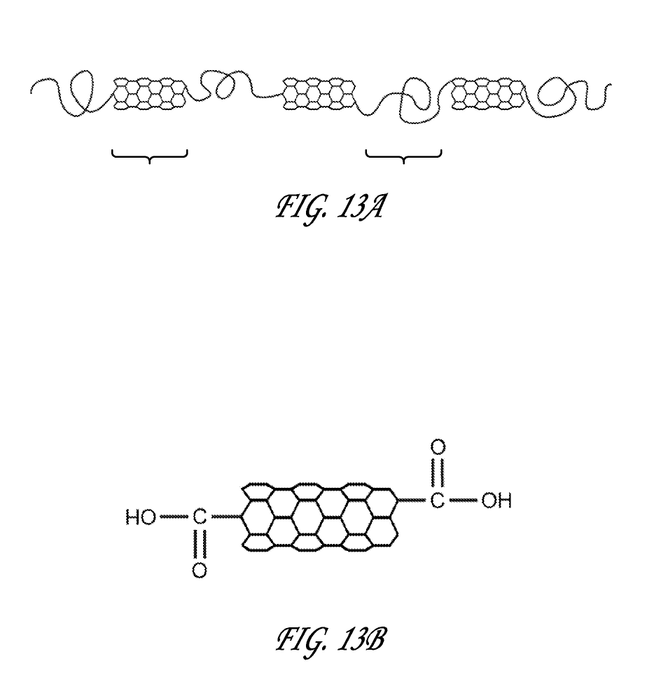

The present invention also provides polymers and copolymers that are composed of carbon nanotube monomeric units. In these embodiments, the polymers and copolymers include a plurality of end-linked single-wall carbon nanotube monomeric units. An example of a copolymer composed of a plurality of end-linked single-wall carbon nanotube monomeric units and other reactive moieties is illustrated in FIG. 13(a), and an example of a single-wall carbon nanotube monomeric unit is depicted in FIG. 13(b). Referring to FIG. 13(a) there is provided a carbon nanotube copolymer 1300 comprising a plurality of single-wall carbon nanotubes 1302 that are end-linked to a plurality of polymers 1304. Referring to FIG. 13(b), the end-linked single-wall carbon nanotube monomeric units 1306 can be provided by opening both ends of a carbon nanotube 1302 and providing at least one covalently-bound functional group 1308 to each of the ends of the carbon nanotubes using a suitable functionalization chemistry as further described herein. In some of the copolymer embodiments, the covalently-bound functional groups at the open ends of the carbon nanotube monomeric units are covalently bonded to one or more reactive moieties (“RM”). Suitable reactive moieties include di- or multi-functional monomers, di- or multi-functional oligomers, and di- or multi-functional polymers. Monofunctional moieties (e.g., monofunctional monomers, monofunctional oligomers and monofunctional polymers) can optionally be included, for example, to control the degree of polymerization by acting as an end-capping agent as known in the art of condensation polymerization. Multifunctional monomers, oligomers, and polymers are useful for preparing cross-linked materials. Nonfunctional moieties can optionally be included in a suitable reaction mixture containing single-wall carbon nanotube monomeric units, for example, to prepare composite materials.

In some embodiments, the monomeric compounds can be other carbon nanotube monomeric units having suitable functional groups that covalently bond with the end-linked single-wall carbon nanotube monomeric units. For example, carbon nanotubes functionalized with carboxylic acid groups and be polymerized with carbon nanotubes functionalized with carbon nanotubes functionalized with amine groups, and variants thereof. Suitable polymerization conditions known in the art of condensation polymerization can be used. Dispersion-based and non-dispersion based condensation polymerization conditions can also be used. The resulting macromolecules can be referred to as polymers having a plurality of single-wall carbon nanotube monomeric units, i.e., poly(CNT).

In other embodiments, the reactive moieties do not include carbon nanotubes, and the reactive moieties have suitable functional groups that covalently bond with the end-linked single-wall carbon nanotube monomeric units. For example monomers, oligomers and polymers (denoted by R and R′ below) that are functionalized with carboxylic acid groups, alcohol groups, amine groups, or any combination thereof are known in the art of condensation polymerization chemistry:

R—COOH+R′—OH→R—COO—R′+H₂O

R—COOH+R′—NH₂→R—CONH—R′+H₂O

These are suitably linked, optionally with a catalyst, to the single-wall carbon nanotube monomeric units of the present invention. The resulting macromolecules can be referred to as copolymers of single-wall carbon nanotube monomeric units and reactive monomer units, i.e., poly(CNT-co-RM). Suitable copolymers include graft copolymers, block copolymers, star copolymers, star block copolymers, random copolymers, alternating copolymers, dendrimers, and the like, as known in the polymer art. Preferably, condensation polymerization chemistry is used to provide alternating copolymers of CNTs and RMs.

Suitable monomeric compounds for forming the carbon nanotube-containing copolymers may include any of the monomers as herein described. For example, the carbon nanotubes can be end-functionalized with amine groups and reacted under condensation polymerization conditions with di-functional or multifunctional carboxylic acid functionalized monomers. Alternatively, the carbon nanotubes can be end-functionalized with carboxylic acid groups and reacted under condensation polymerization conditions with di-functional or multifunctional amine functionalized monomers. They nanotubes and the monomers may also have the same type of functional groups (e.g., carboxylic acid groups) that can be linked under suitable conditions known in the art of step-growth polymerizations. Various chemical coupling schemes known in the art of condensation polymerization can be used to prepare the carbon nanotube-containing copolymers and polymers of the present invention. End-functionalization of carbon nanotubes can suitably be carried out in an aqueous phase. In certain embodiments, the carbon nanotubes can be end-functionalized after dispersing the carbon nanotubes in a fluid medium, for example, after dispersing the nanotubes with NaDDBS in water. In other embodiments, the carbon nanotubes can be end-functionalized before dispersing the carbon nanotubes in a fluid medium. Methods of dispersing the carbon nanotubes in aqueous medium are described hereinabove, for example, using NaDDBS as a dispersing agent.

Among the copolymers of the present invention, several include a plurality of covalently-bonded repeating groups, at least a portion of the repeating groups comprising functionalized single-wall carbon nanotubes. In these embodiments, the single-wall carbon nanotubes typically comprise at least one open end, and preferably comprise two open ends. The copolymers of the present invention can be suitable mixed with other polymers and reactive intermediates for preparing copolymer compositions, such as composite materials as described hereinabove.

The present invention also provides for compounds that comprise a single-wall carbon nanotube comprising at least one open end and at least one functional group bonded at the open end. Preferably, the single-wall carbon nanotubes comprise two open ends and at least one functional group at each of the open ends. Suitable functional groups are capable of step-growth polymerization, chain-growth polymerization, or both. Suitable functional groups include a carboxylic acid group, an alcohol group, an amine group, an ethylenically unsaturated group, a ring-opening group, or any combination thereof. Compositions of these compounds are suitable provided, for example, in an aqueous or organic fluid medium for providing the compounds in a ready-to-use packaged form, for example, for use as a reactive intermediate in preparing carbon-nanotube containing materials as described hereinabove.

Copolymers of the present invention made using single-wall carbon nanotube monomeric units are useful for fabricating strong composite materials and nano-fibers. In several embodiments, the reactive moiety is composed of a functionalized conductive polymer. Suitable functionalized conductive polymers include any type of poythiophene, for example polymers prepared from 3-hexylthiophene. Another example is poly(ethylene-dioxythiophene) (“PEDT”), which is commercially available from H. C. Starck under Bayer Corp, Pittsburgh, Pa. Other suitable conductive polymers include polyaniline and polypyrrole (both available from RTP company, Winona, Minn.), which are useful in electronic applications. Other suitable conductive polymers include poly(phenylene vinylene) and polyarylene (e.g., polyspirobifluorene), both of which are available from the Aldrich Company, which are useful in light emission applications.

The resulting copolymers provide composites and nano-fibers that are electrically conductive, some of which can be made superconductive. Traditional oxidization methods can be used to introduce carboxylic acid groups (—COOH) to the ends of CNTs as described hereinabove. Through esterification or amidation of the ends' carboxylic groups, isolated SWNTs are covalently bonded with the RMs, such as functionalized polymer chains. A schematic illustration of a resulting copolymer is shown in FIG. 13(a). Optionally, cross-linker can be used to form a carbon nanotube imbedded polymer network. Suitable cross-linkers include multi-functional compounds that are capable of covalently bonding to two or more polymer chains. Various cross-linkers and crosslinking conditions are known in the polymer chemistry art and are suitable for use with the instant inventions described herein. For example, polymers and copolymers having CNT segments can be used to form polymeric materials that are very high strength, electrical conductive, or both.

Copolymers of the present invention that include end-linked nanotubes have broad industry applications. They can be directly used as components of strong, conductive composite and nano-fibers. Several embodiments form materials that are characterized as having a rigid rod structure like liquid crystal (“LC”) diblock copolymers. Embodiments of the nanotube-containing copolymers of the present invention can be used in applications that are similar to those used by LC materials, for example in high strength fibers and composite materials. The unique properties of carbon nanotubes enables the preparation of carbon nanotube-containing LC diblock copolymers having unique properties. For example, copolymers formed from CNT and other hydrophilic reactive materials can self-assemble into lamellar phases at proper concentration. This self-assembly of CNT contained copolymer can be transformed into a nano-filter by burning away the non-CNT part of the copolymer. CNT contained copolymers having high tensile and compressive moduli can also be used as a matrix for the preparation of artificial bone material. CNT contained copolymers can also form vesicles, which have quite a few medical and biology applications, such as drug delivery. CTN-containing vesicles are further described hereinbelow. Since nucleic acids contain amine groups, copolymers of the present invention can be prepared by linking the end-functionalized CNT monomers of the present invention with nucleic acids, such as DNA. Both components individually have utility in mechanical, electrical, biology and mechanical applications. Combining carbon nanotubes and DNA have applications in self assembly of carbon nanotubes for assembling nanoscale devices, such as field effect transistors, nanotube-based logic circuits, DNA computers, electrochemical sensors, composite materials, aqueous dispersions of nanotubes, and the like.

The present invention also provides compositions comprising two or more phases, at least one of the phases comprising dispersed carbon nanotubes. The two or more phases may be hydrophilic, hydrophobic, or any combination thereof as long as the phases are thermodynamically or dynamically capable of remaining separate from each other. Suitable hydrophilic phases include aqueous materials, such as water. Suitable hydrophobic phases include organic compounds that are at least partially immiscible with the hydrophilic phase, such as organic compounds, for example, e polymers, monomers and solvents. In some of these embodiments, at least one of the phases is a fluid. In some embodiments, at least a portion of the carbon nanotubes are dispersed in a fluid phase. The fluid phase that contains dispersed carbon nanotubes can be an aqueous or non-aqueous medium. A suitable aqueous medium for providing dispersed nanotubes in water having a suitable surfactant is described herein above. A suitable non-aqueous medium includes an organic solvent, such as DMF and the like, which is known in the art for dispersing carbon nanotubes.

In one embodiment, the aqueous phase of the two phase composition of the present invention is emulsion-based composition that contains carbon nanotubes. The emulsion-based compositions of the present invention include two or more phases, at least one of the phases having dispersed carbon nanotubes. In certain embodiments, at least one of the phases of the compositions of the present invention includes a fluid. The carbon nanotubes are suitably dispersed in a fluid phase in embodiments of the emulsion-based compositions of the present invention. For example, in certain embodiments, the carbon nanotubes are dispersed in an aqueous medium, and in other embodiments the carbon nanotubes are dispersed in a non-aqueous medium. Preferably, the nanotubes are provided in an aqueous medium using a suitable surfactant according to the various methods of dispersing nanotubes as described hereinabove.

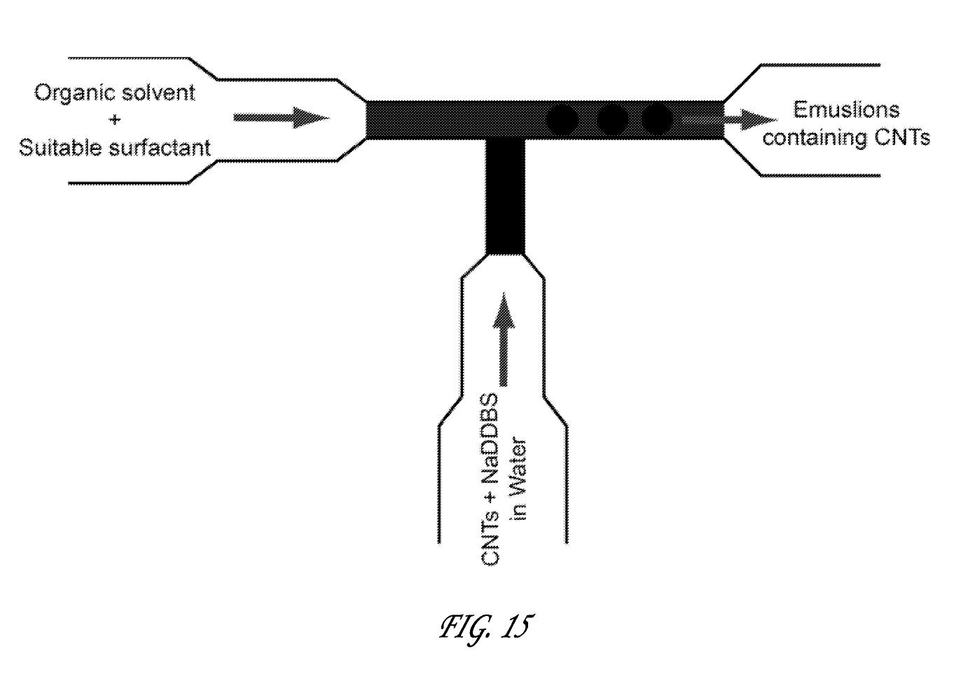

Making emulsions out of carbon nanotubes opens doors to enormous applications of carbon nanotubes. Various emulsions, vesicles and fluid precursors for preparing polymer/nanotube composite materials can be made according to the methods of the present invention. Emulsions, vesicles and fluid precursors can be prepared using the approach shown in FIG. 15 by controlling the compositions and flow rates of the continuous and dispersed phases. Referring to FIG. 15, there is provided a T-channel microfluidic device 1500 suitable for preparing compositions of the present invention comprising two or more phases, at least one of the phases comprising dispersed carbon nanotubes. The T-channel microfluidic device 1500 includes a first fluid conduit 1502 for transporting a first fluid 1504 into microchannel 1506. Also included is a second fluid conduit 1512 for transporting a second fluid 1514 into microchannel 1506 at junction 1520. The first and second fluids are at least partially immiscible so that the second fluid entering microchannel 1506 at junction 1520 forms a droplet of second fluid 1522 in the first fluid 1504. First fluid 1504 becomes the continuous phase 1524 for carrying a plurality of droplets (emulsion particles) of the second fluid 1508 in region 1510 of the microchannel 1506. The droplets of the second fluid 1508 dispersed in the continuous phase 1524 forms a composition comprising two or more phases 1518 that exits region 1510 and enters the two-phase fluid conduit 1516. Suitable dimensions of the conduits and microchannels of the microfluidic T-channel device can be in the range of from about 1 micron to about 1,000 microns, preferably in the range of from about 5 microns to 200 microns, and even more preferably in the range of from about 10 microns to about 100 microns. In certain embodiments, T-channels having 20 micron×20 micron dimensions are suitably used. Each conduit can vary in dimension, for example, from about 100 microns or more distant from the microchannel 1506 to about 20 microns or smaller of the microchannel 1506. The size distribution of the emulsion particles 1508 can be controlled by controlling the volumetric flow rates and channel dimensions of the fluids entering the junction 1530 of the T-channel. The emulsion particle size distribution can be monodisperse, bimodal, trimodal or polymodal.

Suitable T-channels as illustrated in FIG. 15 can be made of polydimethylsiloxane (“PDMS”) using soft imprint technology, the details of which are known in the art. For example, PDMS based T-channel microfluidic devices can be fabricated using soft imprint lithography according to the methods generally described in U.S. Patent Application Pub. No. 2001/0029983 to Unger et al., published Oct. 18, 2001, the portion of which pertaining to the production of T-channel microfluidic devices is incorporated by reference herein. A PTFE channel mold is constructed, and then a PDMS imprint out the channel mold is made. By flowing in organic solvent with suitable surfactant from one inlet and aqueous NaDDBS dispersed CNT solution from the other one, monodispersed CNT contained emulsions 1518 can be prepared in the two-phase fluid outlet 1516.

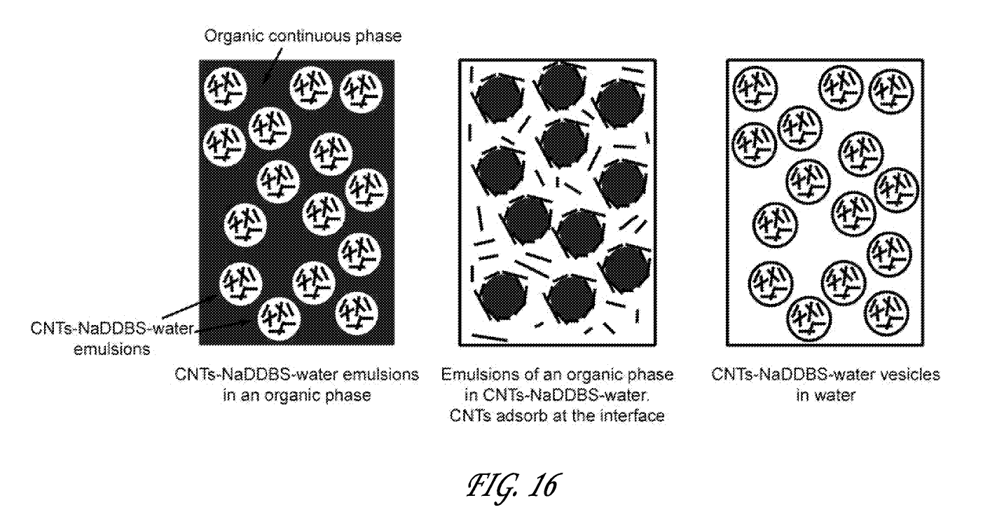

CNT based emulsions have many uses. For example, if monomer can be used as the organic phase for fabricating (water+surfactant+CNT)/monomer inverse emulsions. Adding proper initiator to the monomer phase will initiate the polymerization and a porous CNT encapsulated polymer foam can be produced. Monomer/(water+surfactant+CNT) direct emulsions can also be produced. Because of the hydrophobic effect, CNTs tend to stick to the interface between monomer phase and (water+surfactant+CNT) phase. Adding proper initiator will produce CNT contained colloidal dispersion. Another useful phenomenon is that one phase can be prepared with (water+high weight % surfactant) and the other phase with (water+low weight % surfactant+CNT). Surprisingly CNT contained vesicles can produced this way. The resulting two-phase compositions of the present invention are illustrated in FIGS. 16(a), (b) and (c).

In one embodiment, a suitable microfluidic device comprising a T-channel, such as the one illustrated in FIG. 15, is used to synthesize emulsion droplets containing NaDDBS dispersed carbon nanotubes in an aqueous fluid. In one embodiment, emulsion particles comprising CNTs, surfactants and aqueous fluid can be dispersed in an organic phase using a suitable T-channel to form a two phase composition as depicted in FIG. 16(a). A aqueous phase of emulsion particles of CNTs+NaDDBS+water 1604 are shown dispersed in the organic continuous phase 1602. Any suitable composition of individually dispersed carbon nanotubes in an aqueous fluid can be used, and preferably the aqueous carbon nanotube dispersions described hereinabove are used. The organic phase can be any type of hydrophobic phase that is at least partially immiscible with water. Suitable organic phases include monomer fluids, such as any of the ethylenically unsaturated or functionalized monomers that are capable of polymerizing into polymeric materials. Suitable monomers are described hereinabove. Accordingly, suitable organic compositions (e.g., monomer) are capable of hardening into a composite material (e.g., by using styrene as the organic “solvent” phase). Photoinitiator can be added to the continuous phase to initiate polymerization and lock-in the CNT-containing aqueous emulsion particles in a solid-like phase of polymerized monomer, (e.g., polystyrene (“PS”)) to form a polymer-carbon nanotube (“Poly-CNT”) composite material (e.g., a polystyrene-carbon nanotube (“PS—CNT”) composite material). The Poly-CNT is pressed above the glass transition temperature (“Tg”) of the polymer to eliminate water and air bubbles. Crosslinker is added to the liquid-like composite material to create a crosslinked Poly-CNT composites. Preferably, the polymer is first pressed above its Tg prior to adding crosslinker to prepare composites that are mechanically tough. If crosslinker is added to the polymer prior to heating above Tg, then there will be no glass transition temperature. In this case, water may be removed by heating, but this can create cavities within the polymer matrix which tends to result in brittle Poly-CNT composites.

In another embodiment, emulsion droplets of monomer and photoinitiator can be prepared in a continuous phase of CNT+NaDDBS+water using a suitable T-channel device, such as the one illustrated in FIG. 15. CNTs 1608 and NaDDBS (not shown) from the aqueous phase 1606 adsorb on the monomer emulsion particles 1616 as shown in FIG. 16(b). Polymerization of the monomer in the dispersed monomer emulsion particle phase is initiated using a suitable photoinitiator to prepare polymer-CNT composites. Water and air bubbles are removed from the composite materials as described hereinabove, crosslinker is added to the polymer phase, and the composite material is hardened.

In yet another embodiment, aqueous nanotube (e.g., CNT+NaDDBS+water) emulsion particles are prepared in a continuous oil phase comprising an oil and a surfactant. Any type of oil and oil-soluble surfactant can be used that are suitable for forming water and oil emulsions. A particularly preferred oil is a silicon oil and a preferred oil surfactant is “Span 80”™. In this embodiment a first fluid aqueous phase is provided that includes water, an aqueous phase surfactant, and a water soluble monomer. Suitable surfactants include any of the surfactants described herein for preparing aqueous dispersions of carbon nanotubes. Particularly preferred surfactants include NaDDBS, Tween 20™, Tween 60™, and the like. Suitable water-soluble monomers include any monomer that is miscible in water. Preferred water soluble monomers include ethylene glycol, urethane, n-isopropyl acrylamide, and the like. The continuous oil phase and an aqueous emulsion particle dispersion of nanotubes, surfactant and water are added to the first fluid aqueous phase. Without being bound by any particular theory or method of operation, it is believed that the aqueous phase surfactant molecules migrate to the interface between the continuous silicon oil and water phases. The lighter silicon oil phase stays above the heavier water phase. However, heavier water emulsions slowly sediment through the silicon oil phase and cross the interface between the oil phase and the water phase. The aqueous emulsion particles comprising the carbon nanotubes pick up a second layer of surfactant as they pass through the interface and turn into vesicles. The silicon oil phase is subsequently removed, and photoinitiator is added to initiate polymerization in the continuous water phase. FIG. 16(c) illustrates the resulting vesicles 1612 containing an aqueous phase of dispersed carbon nanotubes 1614. The vesicles 1612 are dispersed in the aqueous phase 1610 that contains the water-soluble monomer (not shown). Alternatively, water soluble monomers and photoinitiator can be added in the water emulsions and simultaneously induce polymerization in both the dispersed water phase inside the vesicles and the continuous water phase. A similar procedure is followed as described hereinabove to remove water and air bubbles/cavities and crosslink the Poly-CNT compositions to created hardened composites materials.

The present invention also provides methods for controlling the deposition of carbon nanotubes on substrates. These methods include providing a patterned substrate comprising a polymer layer and exposed surface features; bonding charged linker molecules, linker molecules capable of being charged, or both, to said exposed surface features; removing said polymer layer; optionally charging the linker molecules capable of being charged; and bonding charged carbon nanotubes to the charged linker molecules, wherein the charge of the charged carbon nanotubes is opposite the charge of the charged linker molecules bonded to the exposed surface features.

The present invention also provides methods for controlling the deposition of carbon nanotubes on substrates. These methods include providing a patterned substrate comprising a polymer layer and exposed surface features, bonding positively charged linker molecules or linker molecules capable of being positively charged to said exposed surface features, removing said polymer layer, optionally positively charging the linker molecules capable of being positively charged, and bonding negatively charged carbon nanotubes to positively charged linker molecules that are bonded to the exposed surface features. The negatively charged carbon nanotubes are conveniently provided using any suitable surfactant that adsorbs to carbon nanotubes and provides a negative charge. Preferred surfactants are provided herein above and have one or more negatively charged head groups as described hererinabove. Particularly preferred surfactants include NaDDBS and the like described hererinabove.

The deposition process of the present invention is conveniently provided on a substrate made of any material on which exposed surface features are provide. Suitable materials includes metals, polymers, ceramics, glass and silicon. Preferably the exposed surface features are composed of silicon dioxide.

Suitable polymer layers include any polymeric material that is capable of being patterned, such as by using one or more micro- or nano-lithographic techniques. Examples of suitable micro- and nano-lithographic techniques include photolithography, E-beam lithography, nanoimprint lithography, and dip-pen nanolithography. Preferably, E-beam lithography is used with a acrylic polymer resist material that is known in the art of E-beam lithography.

Suitable linker molecules are capable of covalently bonding to the substrate, and providing a positive charge to which a negatively charged carbon nanotube is capable of bonding. Examples of suitable linker molecules that are positively charged or capable of being positively charged include a surface linking group and a positive charge group located at least several atoms away from the surface linking group. Suitable surface linking groups include silanes, and the like. Suitable positive charged head groups include —NH₃⁺ ammonium groups, and the like. A preferred linker molecule capable of being positively charged is aminopropyltrimethoxysilane (“APTS”) that has its amine group converted to a positively charged ammonium group by treating with a suitable acid such as hydrochloric acid.

Suitable exposed surface features are characterized as having one or more dimensions smaller than about 1000 nm, more typically smaller than about 500 nm, more typically smaller than about 250 nm, and even more typically smaller than about 100 nm. The exposed surface features can have any suitable shape or geometry, for example the exposed surface features can include trenches, contacting regions, pads, lines, ridges, points, reaction wells, channels, plateaus, or any combination thereof. The exposed surface features are preferably in designed to form an electric circuit or electronic device. In one embodiment of the present invention, the linker molecules self assemble on the exposed surface features.

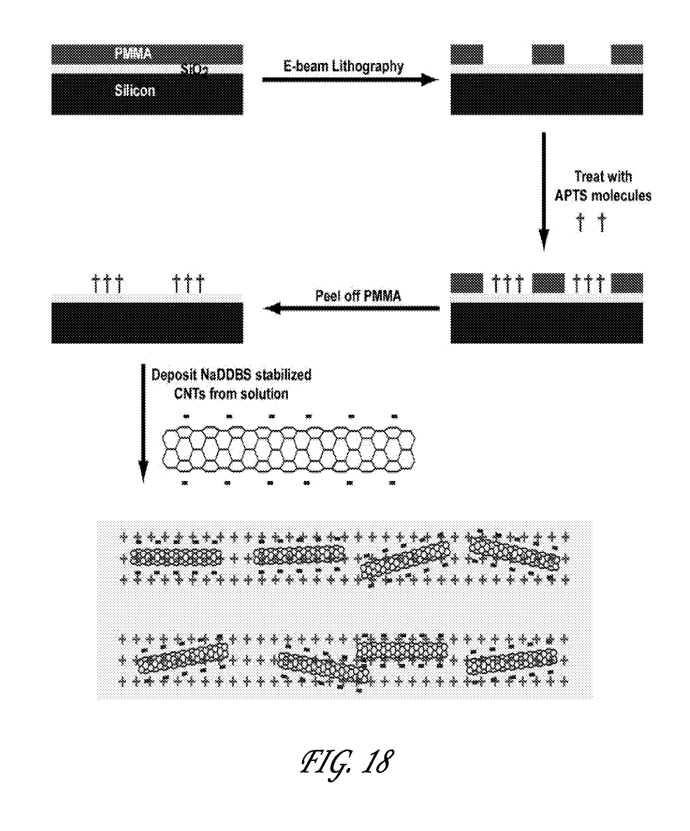

An embodiment of the process of controllably depositing nanotubes on a patterned substrate is provided in FIG. 18. In this embodiment, a substrate 1806, such as a silicon wafer, is surmounted with an oxidized layer 1804 (SiO₂) and a polymer layer (PMMA) 1802. The polymer layer 1802 is patterned using E-beam lithography to provide a patterned polymer layer 1808 and exposed surface features 1820. The exposed surface features 1820 are treated with linker molecules that are positively charged or capable of being positively charged 1810 that link to the exposed surfaces of the oxidized layer 1804 (here, SiO₂). The linker molecules capable of being positively charged can be applied as neutrally charged molecules that are subsequently converted to a positively charged state. For example, APTS has an —NH₂ amine group that can be converted to a positively charged —NH₃⁺ ammonium group by reaction with a suitable acid, for example, fuming HCl. The patterned polymer (PMMA) 1808 is removed to provide a pattern of positively charged linker molecules 1810 linked to the oxidized layer 1804. Negatively charged carbon nanotubes 1812 are deposited to the top surface of the substrate comprising the remaining positively charged linker molecules 1810 and exposed oxidized layer 1804. Negatively charged carbon nanotubes 1812 are suitably provided using an aqueous dispersion of nanotubes using a suitable surfactant having a negatively charged head group, preferably NaDDBS. Without being bound by any particular theory of operation, the negatively charged carbon nanotube 1812 binds selectively to the positively charged linker molecules 1810. As a result, carbon nanotubes are controllably deposited on a substrate. This process can be used to form various types of devices, including circuits, molecular wires, sensors, detectors, logic elements, and the like.

The present invention is also directed to substrates that comprise a surface feature; and a carbon nanotube controllably deposited on the surface feature. The carbon nanotubes can be controllable deposited on the surface feature according to the methods described hereinabove. Substrates according to the present invention have uses in a number of different applications, for example devices, electronic circuits, molecular photon emitters, sensors, single molecular electronic circuits, and the like. In certain embodiments, the substrates further comprise one or more surfactants bound to the carbon nanotube. In other embodiments, the substrates further include a macromolecule bound to one or more of the surfactants on the carbon nanotube. Suitable macromolecules include a nucleic acid or a protein.

In other embodiments, the substrates that comprise a surface feature and a carbon nanotube controllably deposited on the surface feature can further include a microfluidic channel adjacently positioned to the surface feature. In these embodiments, the surface feature can include channels smaller than 1000 nm wide. These embodiments are useful, for example, in preparing carbon-nanotube based microfluidic sensor devices.

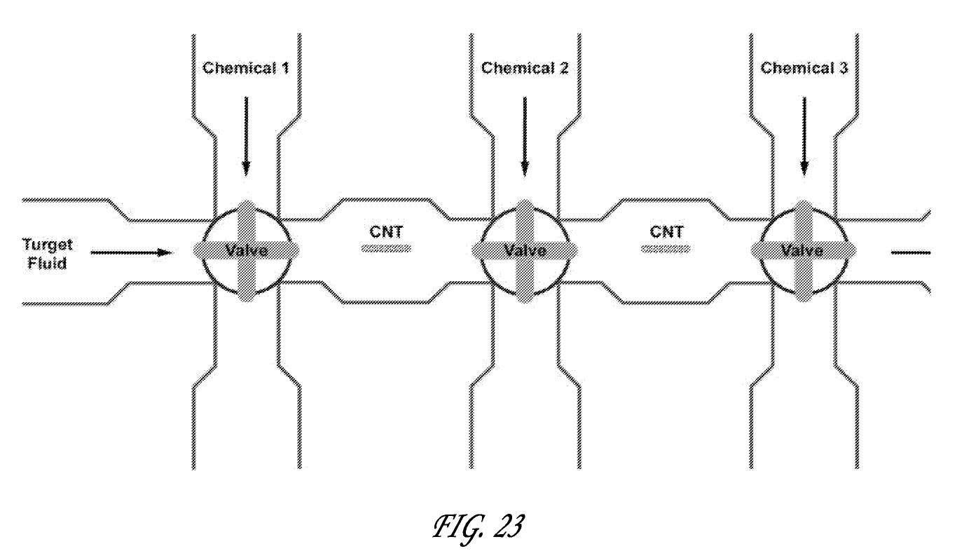

The present invention also provides devices, comprising a substrate fluidically sealed to a microfluidic assembly, the substrate comprising negatively charged carbon nanotubes adsorbed on one or more negatively charged regions on a surface of the substrate; the microfluidic assembly comprising one or more contacting regions adjacently positioned to the substrate for controllably contacting one or more molecular components to said carbon nanotubes; one or more target fluid conduits capable of supplying one or more target fluids comprising one or more analytes; one or more detecting molecule conduits capable of supplying one or more detecting molecules for detecting said analytes in the target fluids; one or more valves capable of directing said target fluids and said detecting molecules into said contacting regions; and optionally one or more exit conduits. FIG. 23 is a schematic illustration of an embodiment of a substrate comprising carbon nanotubes 2314, 2330 that are controllably deposited on a substrate (not shown) and includes a microfluidic assembly to form a carbon-nanotube based microfluidic device 2300. The substrate and microfluidic assembly (not separately shown) are two halves that fluidically sealed together to form the carbon-nanotube based microfluidic device 2300. First and second contacting regions 2318, 2332 in the microfluidic assembly are adjacently positioned to the substrate for controllably contacting one or more molecular components to carbon nanotubes that are bonded to the substrate. Target fluid conduit 2304 provides a suitable target fluid, such as a fluid containing analytes for detection. A first conduit 2308 supplies liquid 2306 containing a first detecting molecule (chemical 1) for detecting a first analyte. Valve 2312 directs fluid 2306 into a first contacting region 2318 to form fluid 2316. First detecting molecules present in fluid 2316 absorb on one or more CNTs 2314 that are controllable deposited on the substrate in the first contacting region 2318. Fluid 2316 can exit the first contacting region 2318 through valve 2324 and exit conduit 2326. Similarly, a second conduit 2322 supplies liquid 2320 containing a second detecting molecule (chemical 2) for a second specific analyte. Valve 2324 directs fluid 2320 into a second contacting region 2332 to form fluid 2328. Second detecting molecules present in fluid 2328 absorb on one or more CNTs 2330 that are controllable deposited on the substrate in the second contacting region 2332. Fluid 2328 can exit the second contacting region 2332 through valve 2338 and exit conduit 2340. The CNTs in the first and second contacting regions (2314, 2328) each detect a separate analyte. A target fluid 2302 containing unknown analytes is fluidically transported into each contacting region (2318, 2332) containing CNTs (2314, 2328) and detected by any of a variety of optical or electrical methodologies. Excess fluids can exit through exit conduits 2310, 2326 and 2340. Valves 2312, 2324 and 2338 are operated to control the flow of fluids through the device.

In another embodiment, the controlled deposition nanotube microfluidic device generally illustrated in FIG. 23 can be used as a biosensor. For example, the biosensor can be used to detect whether certain protein/agents exist in a fluid or their relative concentration. Accordingly, in this embodiment, first conduit 2308 supplies liquid 2306 containing antibodies (chemical 1) for a first specific protein. Valve 2312 directs fluid 2306 into a first contacting region 2318 to form fluid 2316. Antibodies present in fluid 2316 absorb on one or more CNTs 2314 that are controllable deposited on the substrate in the first contacting region 2318. Fluid 2316 can exit the first contacting region 2318 through valve 2324 and exit conduit 2326. Similarly, a second conduit 2322 supplies liquid 2320 containing antibodies (chemical 2) for a second specific protein. Valve 2324 directs fluid 2320 into a second contacting region 2332 to form fluid 2328. Antibodies present in fluid 2328 absorb on one or more CNTs 2330 that are controllable deposited on the substrate in the second contacting region 2332. Fluid 2328 can exit the second contacting region 2332 through valve 2338 and exit conduit 2340. The CNTs in the first and second contacting regions (2314, 2328) each detect a separate protein. A target fluid 2302 containing unknown proteins labeled for detection (e.g., fluorescently labeled) are is fluidically transported into each contacting region (2318, 2332) containing CNTs (2314, 2328). Detection of proteins that are specifically absorbed to the CNTs in one or more of the contacting regions gives rise to identification of proteins, protein concentration, or both, in target fluid 2302. Proteins are identified by observing which antibody binds proteins. Protein concentration is determined by intensity of the detected signal, for example by providing a plurality of bound antibodies in each of the contacting regions. A major issue in biological molecule detection is the ability to analyze very small sample volumes. Accordingly, the advantages of the methods and sensors of the present invention that use only very small amounts of sample target fluids is readily seen.

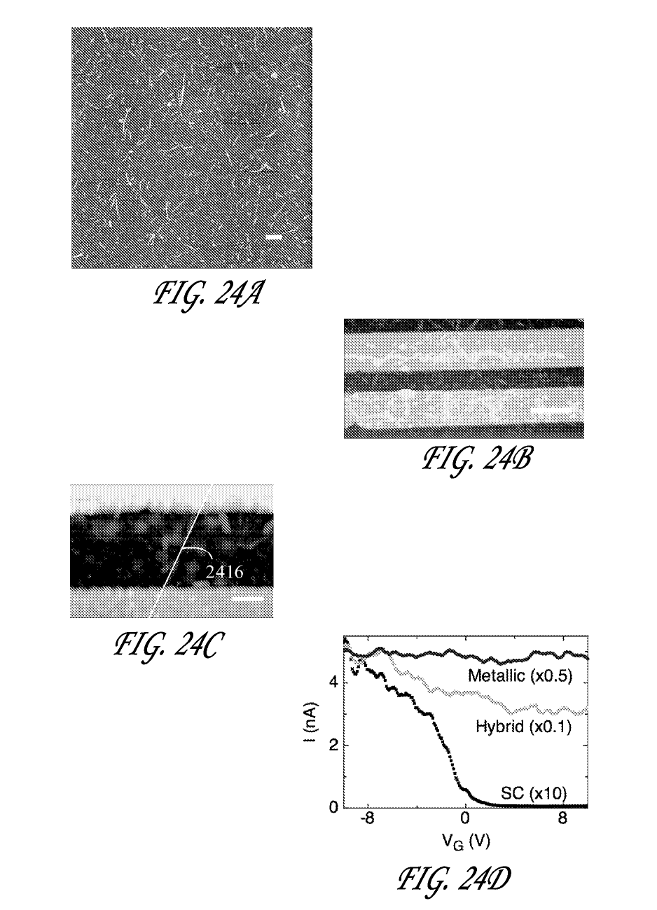

In additional embodiments of sensors of the present invention, electrical conductivity can also be used to detect if any protein had adsorbed onto CNTs by additionally including a pair of electrodes that are electrically connected to the nanotubes. Electrodes (not shown) can be deposited within one or more of the contacting regions (2318, 2332) as described hereinbelow in FIG. 24. The electrical conductivity of carbon nanotubes varies with the presence and amount of absorbed molecules. Measuring variations in electrical conductivity is a preferred method because electrical detection of the biomolecules is more sensitive than optical detection. Accordingly, adsorption of protein molecules onto CNTs changes the charge concentration near the CNTs. The change in the charge localization is detected electrically, which is correlated to detection of the adsorbed proteins.

The present invention also provides processes that include providing an aqueous carbon nanotube dispersion comprising water and individual, dispersed, carbon nanotubes; and chromatographically separating the carbon nanotubes. In one embodiment, the processes include further comprising sequentially removing elutes of the separated carbon nanotubes to form carbon nanotubes having a narrower length distribution (i.e., polydispersity) than the carbon nanotubes provided in the aqueous carbon nanotube dispersion. The present invention can be used for making essentially monodisperse carbon nanotubes.

Most carbon nanotube dispersions are polydisperse (i.e., with respect to length, diameter, chirality, and type). It is desirable to fractionate these polydisperse carbon nanotube dispersions. The first step is to fractionate by length. Dispersions that are monodisperse by length will then be easier to further fractionation. In one embodiment, the present invention uses a gel-exclusion chromatography method to separate the carbon nanotubes by their length. This embodiment is based on two steps. The first step uses a suitable dispersing agent (e.g., NaDDBS surfactant) in the aqueous phase to prepare dispersions of individual CNT in aqueous solutions to form “isolated CNT solutions”. The second step passes the isolated CNT solutions through a gel chromatography column, such as a size-exclusion gel. The longer nanotubes diffuse faster through the size-exclusion gel. Thus, by sequentially taking out elutes from the chromatography column, the CNT solutions can be fractionated by nanotube length.

Dispersions of individual CNT in aqueous solutions to form “isolated CNT solutions” are suitably provided as described hereinabove. Preferably, a 0.1 wt % NaDDBS dispersed CNT dispersion is provided. Any type of dispersing equipment know in the art of preparing particle dispersions can be used, e.g., a mortar and pestle can be used for small amounts, and industrial-scale dispersing equipment can be used for larger amounts. A small amount of surfactant is preferably added to the solution to help to disperse the CNT. After dispersing, more water and surfactant is added and further dispersed. Preferably, the dispersion is suitably sonicated for some time to provide isolated CNT solutions in water.

Chromatography is used to separate the isolated CNT dispersion. Any type of liquid-phase chromatographic method can be used, with size exclusion chromatography being preferred. Suitable size exclusion gels and chromatographic equipment are commercially available. A particularly preferred gel is Sephacryl™ S-1000 superfine gel (from Amersham Biosciences). The gel's cutoff pore size is ˜300 nm. For separate CNTS longer than 300 nm, polymerizable monomer can added to the gel beads and polymerized it to form crosslink among the gel beads, thereby producing larger pores. Micron size pores can be produced this way. Standard gel-exclusion chromatography procedures known in the polymer art is adapted herein for fractionating the CNTs. The CNT dispersion can be passed through the gel under gravity or using a suitable chromatographic pump, and collecting the CNT elutes in time sequence. The earlier time sequence CNT elute fractions contain longer CNT.

Monodispersed SWNT dispersion has broad potential applications in both academics and industry. For example, rigorous small ranged length distribution of SWNT makes it much easier to self-assemble SWNT. Monodispersed SWNT dispersion will also exhibit isotropic-nematic phase transition, which can be used as liquid crystal display.

### Examples and Illustrative Embodiments

Examples and illustrative embodiments of the compounds, compositions, processes, devices and methods of use of the present invention are provided herein.

High Weight-Fraction Carbon Nanotube Dispersions. A method to disperse high weight-fraction carbon nanotubes in water is provided in these examples. A novel surfactant for this purpose, sodium dodecylbenzene sulfonate (NaDDBS), having a benzene ring moiety, a charged head group, and an alkyl chain, dramatically enhanced the stability of carbon nanotubes in aqueous dispersion compared to commonly used surfactants, e.g. sodium dodecyl sulfate (SDS) and Triton X-100 (TX100); dispersion concentrations were improved by approximately a factor of one hundred compared to the commonly used surfactants. The method used herein eliminates the need for high power tip- or horn-sonication and repeated centrifugation and decanting. A single step process is used, which includes mixing SWNTs with surfactant in a low-power, high-frequency sonicator. This sonication procedure enhances disaggregation of bundles of aggregated SWNTs with dramatically less tube breakage. Diameter distributions of nanotube dispersions at high concentrations (20 mg/ml), measured by AFM, show that a large number percentage of these nanotubes were SWNTs (about 61±3%). Initial electronic measurements show that this method does not alter the electronic properties of the nanotubes. Single nanotubes prepared by these means in high concentration can be used for creation of novel composite materials, for self-assembly of nanotubes on surfaces and in dispersion, and for use as chemical and bio-sensors in water.

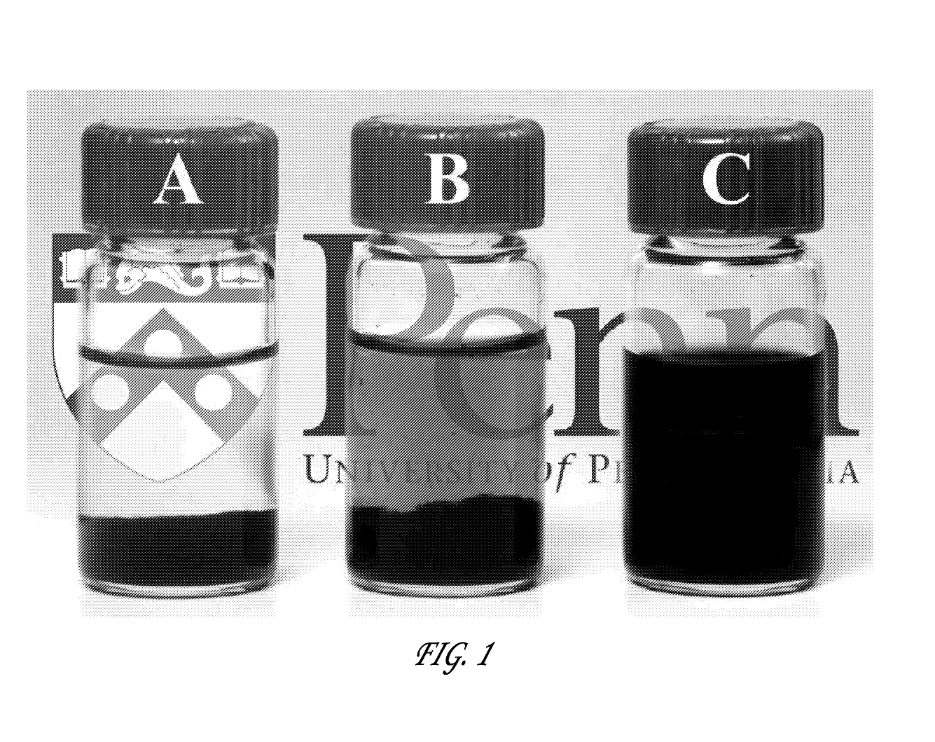

SWNTs were obtained in purified form from Carbon Nanotechnologies Inc. (HiPCO SWNTs, batch 79) and Tubes@Rice (laser-oven SWNTs, batch P081600). According to manufacturer speculations, the HiPCO samples were about 99 wt % SWNTs (0.5 wt % Fe catalyst) and the purified laser-oven nanotubes were greater than about 90 wt % SWNTs. Typically the nanotubes were mixed with surfactant and sonicated in a low-power, high-frequency (12 W, 55 kHz) bath sonicator for about 16 to 24 hours to provide a dispersion. In order to evaluate competing stabilization characteristics, the dispersing power of a range of surfactants was explored: NaDDBS (C₁₂H₂₅C₆H₄SO₃Na), sodium octylbenzene sulfonate (NaOBS; C₈H₁₇C₆H₄SO₃Na), sodium butylbenzene sulfonate (NaBBS; C₄H₉C₆H₄SO₃Na), sodium benzoate (C₆H₅CO₂Na), sodium dodecyl sulfate (SDS; CH₃(CH₂)₁₁OSO₃Na), Triton X-100 (TX100; C₈H₁₇C₆H₄(OCH₂CH₂)ₙOH; n about 10), dodecyltrimethylammonium bromide (DTAB; CH₃(CH₂)₁₁N(CH₃)₃Br), Dextrin, and poly(styrene)-poly(ethylene oxide) (PS-PEO) diblock copolymer. Of the surfactants tested, the dispersions prepared with NaDDBS and NaOBS were by far the most stable; dispersed nanotube concentrations in NaDDBS ranged from 0.1 mg/ml to 20 mg/ml, the highest tested. The resulting dispersions prepared with NaDDBS remained dispersed for at least three months; neither sedimentation nor aggregation of nanotube bundles was observed in these samples. In contrast, highly stable nanotube dispersions could not be prepared with the other additives at concentrations greater than about 0.5 mg/ml. With the exception of NaOBS, a close relative of NaDDBS, reliable disaggregated dispersions in the other surfactants required nanotube concentrations of less than about 0.1 mg/ml. FIG. 1 contains images of the nanotube dispersions in NaDDBS, SDS, and TX100 at 5 mg/ml. The NaDDBS-nanotube dispersion is homogeneous whereas SDS-nanotube and TX100-nanotube dispersions have coagulated bundles of nanotubes at the bottom of their respective vials.

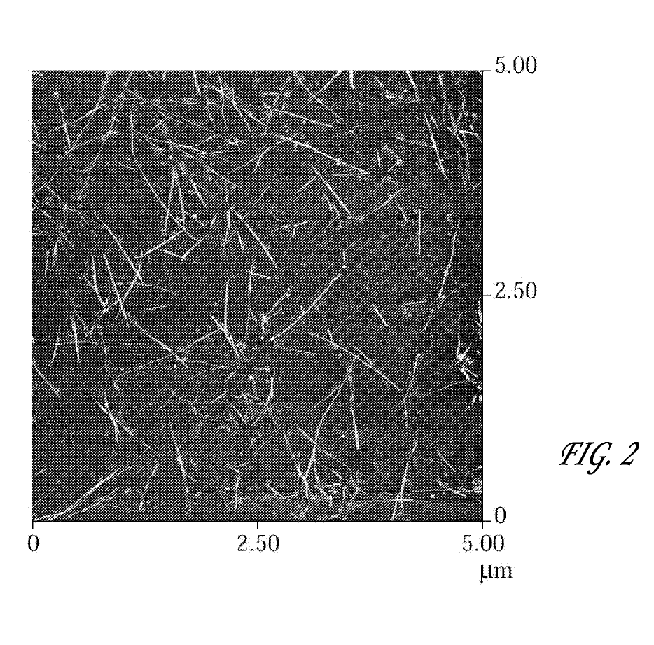

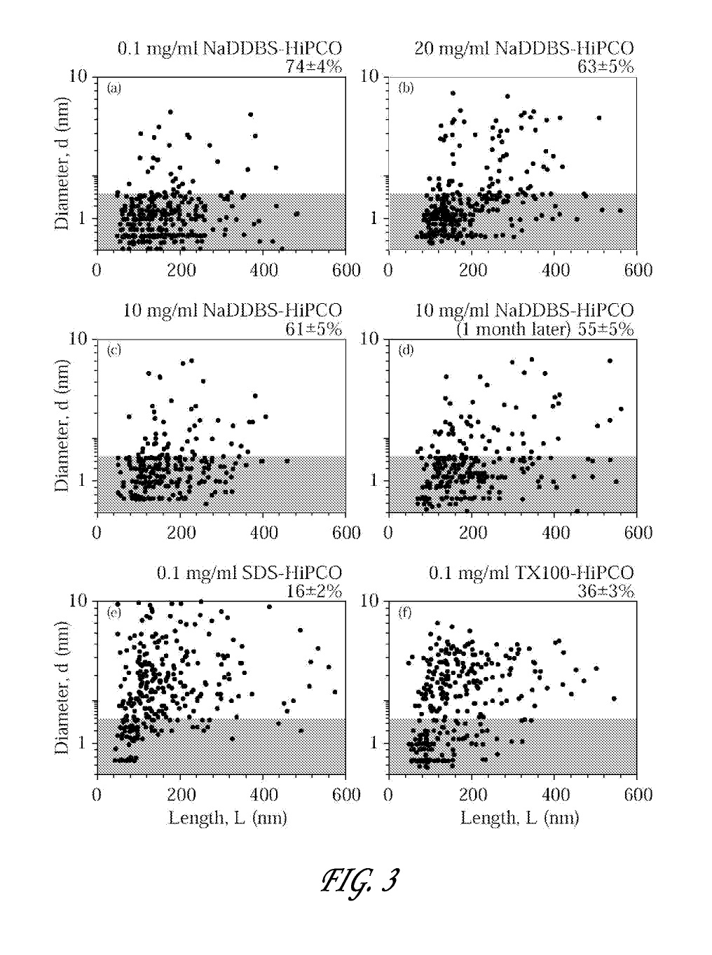

Quantitative information about the distribution of the diameter and length of the dispersed nanotubes was measured using atomic force microscopy (AFM). An example of an AFM image used for this analysis, in this case of laser-oven nanotubes at a concentration of 0.1 mg/ml and stabilized by TX100, is shown in FIG. 2. Surfactant stabilized nanotubes were deposited onto a silicon wafer. The tube surface density was sufficient for analysis when the dispersion nanotube weight fractions were 1 mg/ml; dispersions with greater weight fractions, e.g. above about 20 mg/ml, were rapidly diluted to 1 mg/ml or 0.1 mg/ml and then spread over the silicon wafer for the AFM measurements. The AFM image quality was substantially improved by baking the resultant wafers at 180° C. for approximately 4 hours (or longer); apparently baking removes much of the surfactant from the wafer and from the nanotubes. AFM images were taken in tapping mode using a Nanoscope III Multimode (Digital Instruments Inc., Santa Barbara, Calif.). Digital Instrument supplied software was then used to derive the length and the diameter of the every accessible nanotube in the image. Nanotubes that were not entirely within an image were excluded. Tube diameters were derived from our height measurements, which had a resolution of about 0.1 nm; typically four separate height measurements were made for each tube and were then averaged. Tube lengths were determined within our lateral resolution of about 20 to 50 nm; it was difficult to accurately characterize nanotubes whose lengths were less than 50 nm, so their contributions are not reflected in the measured distributions. A summary of the AFM observations is given in FIG. 3. About 300 nanotubes were examined for each distribution plot. The shaded regions define single nanotubes; 1.3 and 1.5 nm was used as the upper bound for a single tube diameter of the HiPCO and the laser-oven prepared nanotubes, respectively.

The first four distributions are for NaDDBS-HiPCO dispersions. FIG. 3(a) shows that a NaDDBS-HiPCO dispersion prepared at 0.1 mg/ml was about 74±5% single nanotubes. This yield changed modestly as a function of increasing nanotube weight-fraction, see FIG. 3(b) and FIG. 3(c). Furthermore, the distribution from the 10 mg/ml dispersion was measured after allowing it to sit for one month; the single-tube fraction did not change appreciably (about 54±5%; FIG. 3(d)). By contrast, HiPCO stabilized in SDS and TX100 at a concentration of 0.1 mg/ml had SWNT yields of about 16±2% (FIG. 3(e)) and about 36±3% (FIG. 3(f)), respectively. The mean length (Lₘₑₐₙ) of single nanotubes for the four NaDDBS-HiPCO distributions was about 165 nm with a standard deviation between 75 and 95 nm. The number of longer nanotubes (i.e., greater than about 300 nm) was observed to decrease slightly in the samples that were diluted to about 1 mg/ml (distributions not shown). SWNT length distributions for SDS-HiPCO (Lₘₑₐₙ about 105 nm±78 nm), and for TX100-HiPCO (Lₘₑₐₙ about 112 nm±54 nm) were shifted a bit lower; generally many long SWNTs were not found using SDS or TX100.

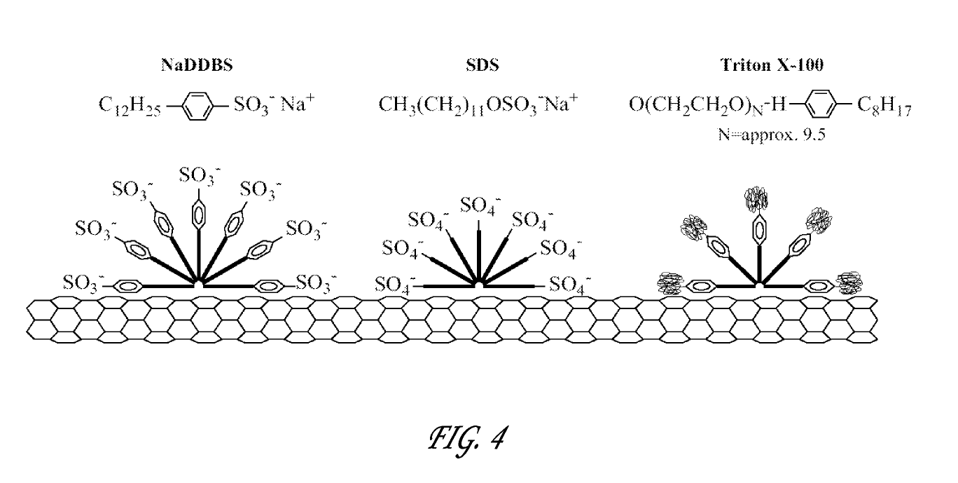

The solubilizing capabilities (i.e., “dispersing power” or “dispersing capability”) of the various surfactants was also investigated. Without being bound by any particular theory of operation, any successful dispersing method must reckon with the substantial van der Waals attractions of bare nanotubes. A schematic of how the surfactants might adsorb onto the nanotubes is depicted in FIG. 4; the nanotubes are stabilized by hemi-micelles that sheath the surface. The superior dispersing capability of NaDDBS compared to SDS (dispersing capability≦0.1 mg/ml) or TX100 (dispersing capability≦0.5 mg/ml) may be explained in terms of graphite-surfactant interactions, alkyl chain length, head group size and charge as pertains particularly to those molecules that lie along the surface, parallel to the tube central axis. It is suspected that SDS has a weaker interaction with the nanotube surface compared to NaDDBS and TX100, because it does not have a benzene ring. Indeed π-like stacking of the benzene rings onto the surface of graphite is believed to significantly increase the binding and surface coverage of surfactant molecules to graphite. Dextrin (dispersing power less than 0.05 mg/ml) and DTAB (dispersing power less than 0.1 mg/ml) also did not disperse nanotubes well because, it is believed, they do not have ring moieties. It is suspected that the alkyl chain part of surfactant molecules lies flat on the graphitic tube surface. Most of the surfactants of the present invention in these examples had alkyl chains with lengths of order 2 nm. Thus, when adsorbing onto a small diameter nanotube surface it is probably energetically favorable for the chains to lie along the length of the nanotubes rather than to bend around the circumference. This chain interaction distinguishes TX100 (8 carbon alkyl chain) from NaDDBS and SDS (both have 12 carbon alkyl chain). Longer chain lengths improve surfactant energetics, given similar ring and head groups. For example, sodium benzoate (no alkyl chain, dispersing power≦0.01 mg/ml), and NaBBS (4 carbon alkyl chain, ≦0.1 mg/ml) have same ring and head group size as NaDDBS, but did not perform very well because of substantially shorter alkyl chain length. On the other hand, NaOBS (8 carbon alkyl chain, dispersing power up to 8 mg/ml) performed quite well. Sodium hexadecylbenzene sulfonate had a longer alkyl chain (16 carbons), but did not dissolve in water at high concentration (more than about 5 wt %) at room temperature—surfactants having alkyl groups greater than about 16 carbons can be dissolved using elevated temperatures, by the use of solvents that are soluble in aqueous media, or both.

Without being bound by a particular theory of operation, the different responses of NaDDBS and TX100 probably arise from head group and chain lengths. The head group of TX100 (PEO chains) is polar and larger than NaDDBS (SO₃⁻); its large size may lower its packing density compared to NaDDBS. Furthermore, the electrostatic repulsion of SO₃⁻ leads to charge stabilization of nanotubes via screened Coulomb interactions which, in analogy with colloidal particle stabilization, may be significant for dispersion (solubilization) in water compared to the more steric repulsion of the TX100 head group. Generally, added salt (NaCl) of greater than about 25 mM induced aggregation in the NaDDBS samples. PS-PEO diblocks, which had long PEO chains as head group, did not stabilize nanotubes well (≦1.0 mg/ml).

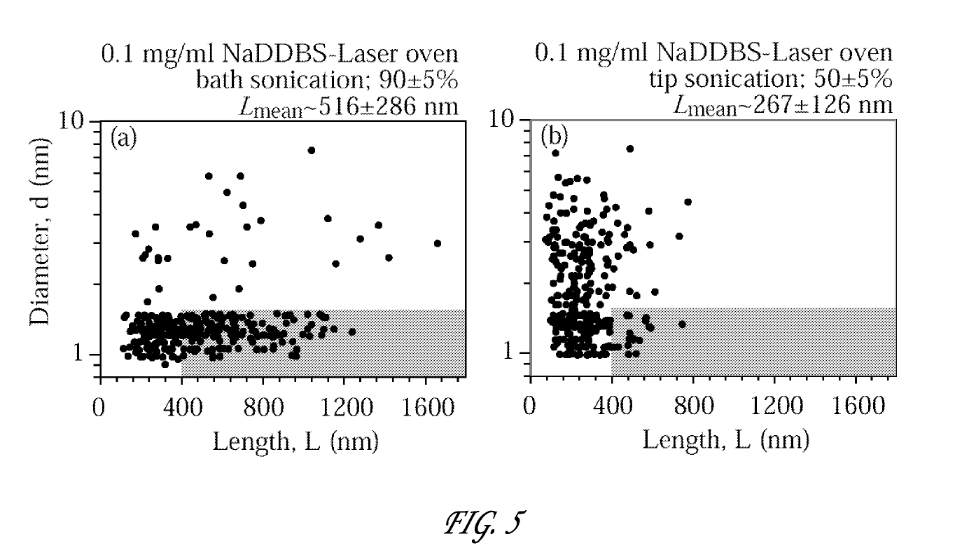

The relative efficacy of different sonication techniques on the dispersion of nanotubes was investigated in the following examples. Tube length is a parameter that is desirable controlled in preparing SWNT dispersions; SWNTs with large lengths (e.g., greater than about 500 nm) are often desirable for introducing greater anisotropies into the properties of composites. The standard approach is to disperse nanotubes using a high power tip sonicator (⅛ inch, 6 W, 22.5 KHz) for short time (about 1 hour). For comparison, 0.1 mg/ml of HiPCO nanotubes and laser-oven nanotubes were prepared in NaDDBS, SDS and TX100, and the resulting length and diameter distributions were measured. Observations of these studies are summarized in FIG. 5 for 0.1 mg/ml laser-oven nanotubes dispersed with NaDDBS. The nanotube dispersion prepared by bath sonication had very high yield (number percentage) of single nanotubes (about 90%±5%), a significant percentage of which were long single nanotubes with lengths longer than about 400 nm (Lₘₑₐₙ about 516 nm±286 nm), see FIG. 5(a). Similar samples prepared by tip sonication (FIG. 5(b)) had lower single SWNT yield (about 50%±4%), and Lₘₑₐₙ about 267 nm±126 nm. These effects were not as pronounced in the HiPCO nanotube dispersions, apparently for the reason that the HiPCO nanotubes were already rather short compared to the laser-oven nanotubes.

Uses of High Weight Fraction SWNT Dispersions. The 100× increase in nanotube solubility, and the relatively smaller amount of tube fragmentation, makes a plethora of processing schemes for SWNTs more accessible, as listed here:

Preparation of Composites: there is Great Interest in Manufacturing Composite materials with large tensile and torsional strength or better thermal or electrical properties. Long and relatively non-fragmented nanotubes can be readily incorporated into any polymer matrix to increase both the tensile and torsional strength, change thermal or conducting properties using the methods described herein. For example, nanotube dispersions were mixed with epoxies to improve the thermal property of epoxies. Here, a commercially-available epoxy emulsion (EPON-3510-W-60, Shell Chemical, emulsion of bisphenol-A epoxy dispersed in water, 60 wt. percent active solids) was mixed with a 20 mg/ml aqueous SWNT dispersion made according to the methods described above. 1 ml of the epoxy emulsion and 200 microliters of the nanotube dispersion was sonicated at 80° C. using the above-described methods. Evaporating off the water provided 600 microliters of a liquid dispersion containing epoxy and the nanotubes dispersed therein. Curing agent (EPICURE-3234, Shell Chemical; EPICURE-9553, Shell Chemical) was mixed into the epoxy-nanotube dispersion, and curing was carried out by the “SONICURE”™ (University of Pennsylvania, Philadelphia, Pa.) process. This process incorporated the simultaneous sonication and curing of the dispersion. This process provides localized heating and curing of the mixture (i.e. gel precursor), which hardens in about two to three minutes. The SONICURE™ process advantageously helps to maintain the nanotubes dispersed in the epoxy resin during curing. The resulting nanotube composite was annealed for two hours at 120° C. to provide a composite having a solid matrix and the carbon nanotubes dispersed therein. In related examples the epoxy-nanotube-curing agent mixture was simply heated to 120° C. for curing.

Electrical Conductivity of SWNT-Epoxy Composites: The electrical conductivity of a SWNT-epoxy composite material made according to the above procedure was about 10⁻⁵ S/cm. The composite contained a concentration of about 0.05 mg of nanotubes dispersed per ml of composite material. Notably, this electrical conductivity is about 100 times larger than the value of the nanotube epoxy composite reported by Park et al., Chem. Phys. Lett. Vol. 364, page 303, 2002, which had between 2 mg nanotubes per ml of composite material.

Self-assembly to form a SWNT Monocrystal: Now that stable dispersions of nanotubes can be prepared at high concentration, nanotubes can be assembled into 3-D crystals using graphite surfaces as templates and depletion interactions or convection as the driving force. Using convection as a driving force and graphite as a template, a single layer of highly organized pyrolytic graphite (“HOPG”) strip was affixed to a glass cover slip. A SWNT dispersion made according to an earlier example was placed in a 5 mm diameter×4 cm vial. The HOPG strip/cover slip was dipped into the dispersion in the vial at an angle. This assembly was placed in an oven at 50° C. to allow the water to evaporate. Capillary force apparently organized the nanotubes at the liquid-vapor interface as the water evaporated. Evaporation was completed after 4-5 days. This process initially provided a monolayer of nanotubes at the beginning of the evaporation process. Towards the end of the evaporation process, a large monocrystal of self-assembled nanotubes having a thickness greater than about one millimeter was formed. This monocrystal can be used in memory devices and display units.

Length, chirality sorting and purification: Nanotubes of the present invention that are well covered by anionic surfactants in dispersion can be post-processed using electrophoresis to separate the nanotubes by length. The adsorbed surfactant molecules can function as “handles” that drag the nanotubes along the field through the electrophoresis gel. This method can also separate the nanotubes from impurities. Thus, this adsorption mechanism of the alkyl group of a surfactant can be used to sort armchair nanotubes (which are typically metallic) from zigzag or chiral nanotubes. Length sorting was carried out in a gel having large pore sizes. A column containing 0.5 percent agarose gel was prepared to provide a large pore size. A vertical column 30-40 cm long was prepared. An aqueous SWNT dispersion made according to an earlier example was poured in the top of the column and the nanotubes were recovered based on length. The size exclusion effect of the agarose gel permitted the longer tubes to exit the column first, thereby effecting separation by length. Narrow nanotube length distributions based on peak length were obtained, e.g., mean lengths of 500 nm+/−20 nm were obtained.

In a related example, the agarose gel is placed between electrodes, and a voltage of about two volts is applied across the electrodes to assist the separation of the nanotubes in the vertically-oriented column. In a horizontally-oriented column, separation is effected by placing electrodes at the column ends and applying a voltage of about 5-10 volts.

Controlled deposition on surfaces: Carbon nanotubes are controllably placed on surfaces (e.g., positively charged silicon wafer) at any specific location using the aqueous nanotube dispersions as prepared in one of the earlier examples. The negatively charged surfactants enable circuit design with carbon nanotubes. A silicon wafer is patterned using any one of the known methods (e.g., via photoresist microlithographic methods) suitable for preparing a positively charged pattern on a substrate. The aqueous dispersion is coated onto the substrate and the nanotube-surfactant moieties adhere to the positively charged pattern. After deposition, the surfactants on the tube can readily be vaporized by baking the resultant wafer at 180° C. to leave patterned nanotubes on the substrate surface.

Controlled deposition of the nanotubes is carried out as follows: A silicon wafer is coated with a suitable photoresist coating (e.g., acrylic-based polymer solution with a UV-activated initiator), and then patterned using e-beam or light. Depending on whether the photoresist is positive or negative, a micropattern is formed by subsequent treatment with solvent to remove the uncrosslinked photoresist. Aminopropyltriethoxysilane (APTS) is then vapor deposited onto the patterned wafer (one to two ml solution of APTS solution in vacuum jar with wafer facing up; evacuate for 30 seconds to deposit APTS on the pattern). After APTS deposition, most of the APTS is removed by sonicating the wafer in DMF to provide a monolayer of APTS on the surface. Presence of the APTS monolayer (0.7 to 0.9 nm thick) is confirmed with ellipsometry or an AFM technique. The wafer is removed and dried and placed in HCl vapor to protonate the amine to form a positive charged surface. The wafer is submerged into nanotube dispersion for 6-12 hours, removed, and rinsed with water. The wafer is dried in a clean environment (ca. 6 hours), and submerged in solvent (e.g., acetone) to remove the photoresist. This procedure provides nanotubes patterned on a substrate, which is used to build circuits and sensors, as described herein.

Chemical and bio-sensors: The surfactants used in the present invention that have a charged head group containing SO₃ can be used for fashioning nanotubes into sensor devices for chemical and biological compounds. Nanotubes respond electronically to adsorption of charged atoms, such as a single hydrogen atom (i.e., a proton). The controlled deposition of carbon nanotubes as described above is carried out with a surfactant having a SO₃ charged head group. The nanotubes are used as is or with slight chemical modification to detect the level of analyte (e.g., NH₃ or NH₂) in a test sample. If the test sample is a portion of the atmosphere then the sensor is suitable for monitoring air pollution or minute contamination. In sensor applications, the surfactant is physically adsorbed to the nanotube surface. The SO₃ group binds chemically to the NH₃ in the sample. A microfluidic device is built containing a circuit that incorporates the SWNTs patterned in a region over which the sample liquid containing the analyte flows. The NH₃ component in a sample is absorbed onto the nanotubes. The nanotubes are connected to electrical contacts in the circuit, and a voltage (V) is applied and the current (I) is measured. When an analyte molecule is adsorbed onto the nanotubes, a change in the current-voltage (I-V) curve is used to detect the presence of a targeted analyte.

A variety of analytes can be detected using this sensor, including hydrogen (i.e., protons), ammonia, amine groups, CO and CO₂. Nanotubes with amine surface groups arising from the surfactants or chemically modified nanotubes can easily bind to different kinds of biological molecules and be constructed into bio-sensors. Nanotubes dispersed using surfactants having an amine group at the end, e.g., an ammonium group, are useful for binding and detecting biological molecules. In this example, a nanotube dispersion is prepared using a surfactant wherein the charged head group is capable of binding biological molecules (e.g., nucleic acids, proteins, and polysaccharides). For example, the amine form of NaDDBS (i.e., the aromatic group is attached to a chargeable ammonia head group) is used in aqueous solution. Controlled deposition of the nanotubes is carried out in a microchannel device as described earlier. The bound nanotubes in the microchannel device are connected to electrical contacts in an array to monitor a plurality of I-V response curves for a plurality of nanotubes The measured I-V curve of the nanotubes changes depending on the binding of biological molecules, which is used to detect the presence of the same or different biological molecules. Different molecules are detected at different points in the array using different specific ligands attached to the nanotubes. For example, standard hybridization targeting assay techniques using a variety of nucleic acid ligands can be used to specifically detect targeted genetic material of a biological agent.

Creating composites containing nanotubes via sol-gel reaction: Nanotubes were dispersed using a bath sonicator as follows. 10 mg of a 20 mg/ml nanotube dispersion dispersed with 10 mg/ml NaDDBS was added in a crucible to 90 mg of silica gel precursor in water, 40 wt. percent solids weight fraction (DuPont, Wilmington, Del.). The pH was lowered to a value of about 4 by adding HCl. The system formed a ceramic composite gel material after five minutes without any visible macroscopic phase separation of nanotubes.

The ceramic composite gel material is subsequently annealed at elevated temperatures and pressures to provide a ceramic material. Annealing silica gel at 1100 deg C. gives rise to ceramics having nanotube voids as the carbon nanotubes will burn off at this elevated temperature.

In another example, an alumina oxide gel precursor is substituted for the silica gel precursor as described above to provide a composite containing nanotubes dispersed in alumina gel. Alumina oxide gel precursors, 40 weight percent solids in water, are commercially available from DuPont, Wilmington, Del. The pH is lowered and the gel forms. The nanotube-alumina gel composite is annealed at temperature in the range of from about 300 to 450 deg C. At these lower temperatures the carbon nanotubes remain substantially intact to provide ceramic alumina composites containing dispersed carbon nanotubes.

Creating thermoplastics with surfactant stabilized nanotubes in water: Nanotubes were dispersed in water (20 mg/ml dispersed in water using 2:1 nanotubes to NaDDBS surfactant). These dispersions are emulsified in a non-aqueous solvent or oil phase to form aqueous emulsions of carbon nanotubes in non-aqueous phase (e.g., solvent or oil). A non-aqueous phase containing 1.5 wt percent Span 80 (Sorbitan monooleate surfactant, Aldrich Chemical Co. Milwaukee, Wis.) in hexadecane solvent was prepared. The nanotube dispersion was micropumped in one channel of a microfluidic T cell having dimensions of 50 to 100 micron square capillaries as the non-aqueous phase was micropumped into a second channel of the T cell. An emulsion of aqueous nanotube dispersions in a non-aqueous phase was formed at the junction. Flow rates were in the range of from about 100 to 500 microliters per hour for both channels to form microdroplets of aqueous carbon nanotubes in the hexadecane solvent. The microdroplets were between 40 micron and 100 microns, depending on the channel size. The microdroplets were collected in a vessel, allowed to settle, and the excess solvent top layer was removed. Methyl methacrylate (MMA) monomer was dissolved in dimethylformamide (DMF) solvent (2-7.5 wt. percent MMA based on solvent). Ethyleneglycol diacrylate (EGDA) crosslinker, 0.5 to 1.0 wt. percent based on monomer weight, was added to the non-aqueous phase of the collected microdroplets. Polymerization was initiated in the non-aqueous phase using sodium persulfate (0.2 wt. percent based on monomer). Temperature was raised to 60° C. and polymerization continued for about several hours until gelation occurred. The polymer formed a gel matrix with the nanotubes embedded therein. The resulting material was subjected to elevated temperatures and reduced pressures to remove excess solvent and water. A black rectangular solid composite material having dispersed nanotubes in a plastic resin was obtained. This material can be heated above its Tg and the nanotubes oriented as described below.

Overview of Alignment of SWNTs to Provide Nematic nanotube gels. The following examples describe three methods used to align SWNTs inside a gel matrix to prepare nematic nanotube gels. To induce alignment of SWNTs in gels, SWNTs were dispersed at low concentration (≦0.78 mg/ml) in an aqueous N-isopropyl acrylamide (NIPA) gel precursor. Polymerization was initiated by chemical means at 295K. The pre-gel solutions were then loaded into rectangular capillary tubes and allowed to polymerize at 295 K. The polymerization process completed in about 1 hour. In the first example of creating nematic nanotube gels, the gel volume was reduced substantially by increasing its temperature; this volume phase transition arises when the polymer network in the gel becomes hydrophobic and water is expelled (and removed). If the initial nanotube concentration was sufficiently large, then the tubes aligned locally. In the second example, water was slowly evaporated out of the SWNTs-NIPA gel through the open ends of the capillary tubes. The flow of water out of the capillary tubes caused the nanotubes to align along the flow direction of water (the long axis of the capillary tubes). In the third example, the capillary tubes with SWNTs-NIPA gel were placed inside a magnet immediately after the initiation of polymerization for the duration of the polymerization process. The nanotubes were aligned by the magnetic field and were locked in place by the gel. By varying the magnetic field strength, gel viscosity and polymerization time, it was possible to align the nanotubes, make nanotube needles with multiple nanotubes, and make long aligned ropes of nanotubes.

Dispersions of laser-oven SWNTs (obtained from Tubes@Rice) having greater than 90 wt % SWNTs were prepared as described in the previous examples with NaDDBS. These nanotube dispersions had very high yield of single tubes (about 90±5%) with average length Lₘₑₐₙ about 516 nm±286 nm. It is not critical to use laser-oven SWNTs in these examples; HiPCO nanotubes (Carbon Nanotechnologies Inc., batch 79; Lₘₑₐₙ about 165 nm±95 nm) have also been used and similar results were obtained.

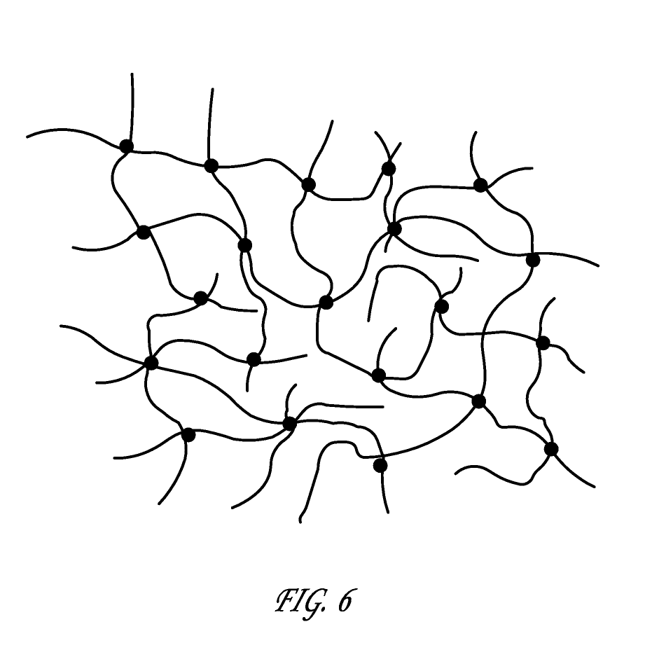

Most, but not all of these experiments used a gel of polymerized N-isopropyl acrylamide monomer (NIPA; 700 mM), N,N′-methylenebisacrylamide (cross-linker agent; 8.6 mM), ammonium persulfate (initiator; 3.5 mM) and N,N,N′,N′-tetramethylethylenediamine (accelerator at 295 K; 0.001% v/v). All components were obtained from PolySciences Inc. (Warrington, Pa.) and were used without further purification. To prepare SWNTs-NIPA gels, SWNT dispersions and all gel reagents were first mixed, except the initiator, in water. The SWNT concentrations ranged from 0.04 mg/ml to 0.78 mg/ml (the NIPA monomer did not gel well when the SWNT concentrations were higher). The gel initiator was then added to the mixture which was then vortexed for 15 seconds. The polymerization took about an hour. The vortexed pre-gel solutions were loaded into rectangular capillary tubes with inner dimensions (length×width×thickness) of about 4 cm×about 4 mm×about 0.2 mm and a wall thickness of about 0.2 mm. FIG. 6 shows a schematic of gel structure after polymerization in the presence of cross-linker.

To align the nanotubes outside of a magnet, the pre-gel solution were polymerized at 295 K for about three hours. To align the tubes inside of a magnet, the capillary tubes with SWNTs-NIPA gel were placed inside a 9 Tesla magnet and at 295 K for longer than the required time for complete polymerization (about 2 hrs). The initial gelation process appeared to lock nanotubes into place, producing a dilute tube distribution with random location. The tube orientation was random when the gel polymerized outside of a magnet; the tube orientation was parallel to the applied field when gel polymerized inside a magnet. Apparently, the tubes could not diffuse over long distances in the gel, but could reorient and move short distances with relatively small energy cost.

Images depicting birefringence in the nematic nanotube gels were obtained with samples situated between crossed-polarizers in a microscope on a rotating stage. The crossed-polarizer measurement is sensitive to birefringence in the sample, which arises when nanotubes align. The aligned regions appear bright in the image; isotropic regions appear dark. For clarity the pass axes for the input and output polarizers were set to be along the x and y directions respectively; the light transmission direction was along z; the sample was rotated in the xy-plane. By rotating the stage, information about the direction of alignment of nanotubes was obtained. By keeping the microscope bulb intensity and the video gain/offset the same for the full set of images, semi-quantitative information about the degree of alignment of nanotubes in the NIPA gel was obtained from the relative intensity differences between various images or regions within an image. An increase in the degree of nanotube alignment was manifested as an emerging bright domain or an increase in the brightness of a domain. Bright-field images of the nanotube needle or ropes in the nematic nanotube gels were obtained without cross-polarizers. The birefringence and the structures within the sample were visualized using a Leica DMIRB inverted optical microscope with a 10×, 5× and 1.6× air objectives. The samples were imaged using a CCD camera (Hitachi, model KP-M1U, 640×480 pixels) and recorded directly into a computer hard-drive using a 8-bit video frame grabber (model CG7, Scion Corporations, Frederick, Md.).

The magnet used to align the nanotubes was a super-conducting magnet (Quantum Design, San Diego, Calif.) for which the magnetic field could be varied between −10 Tesla to +10 Tesla and the temperature could be varied between 4 K and 373 K. To align the nanotubes, the capillary was loaded inside the magnet immediately after the polymerization of NIPA gel was initiated. This remained inside the magnet at 9 Tesla for longer than the duration of polymerization (about two hours).

Method 1: Local alignment of SWNTs by shrinking the SWNTs-NIPA gel. To induce nematic-like structures in SWNTs-NIPA gel, the capillary tubes with SWNTs-NIPA gel were immersed inside glass vials containing 20 mM Trizma buffer (Sigma-Aldrich, St. Louis, Mo.) and placed the entire sample assembly inside an oven at 323 K. The polymer network in the gel became hydrophobic around 323 K. The gel then reduced its volume by expelling water and therefore, the effective volume fraction of the locked SWNTs in the gel increased. For sufficiently large initial nanotube concentrations the tubes aligned locally. The capillary tube containing shrunk SWNT-NIPA gel was taken out of the buffer, the expelled water was removed, and the sample imaged under the microscope. Removal of expelled water prevents the gel from swelling to its pre-shrunk volume as the gel temperature is lowered to room temperature (about 295 K) and the polymer networks became hydrophilic. This local aligning of SWNTs in NIPA gel is referred to herein as a “quasi-isotropic-nematic” transition.

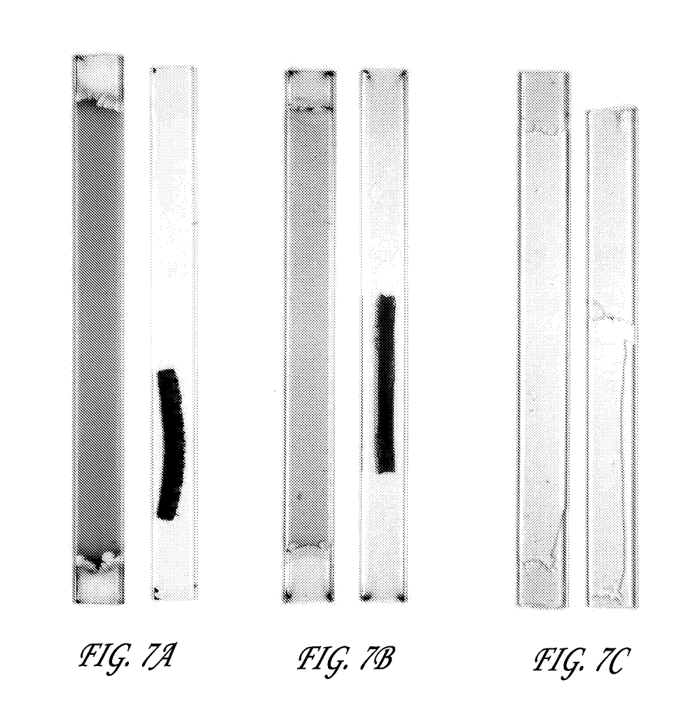

FIG. 7 is a photograph of the SWNTs-NIPA gels and NIPA gels with surfactant alone (7.8 mg/ml NaDDBS) before and after shrinking. Typical pre-shrunk sample dimensions (length×width×thickness) were about 4 cm×about 4 mm×about 0.2 mm and shrunk dimensions were about 2 cm×about 2 mm×about 0.1 mm. The sample in FIG. 7(a) had a high initial nanotube concentration (0:78 mg/ml) and the material underwent a quasi-isotropic-nematic transition immediately after shrinking. The sample in FIG. 7(b), by contrast, initially contained a dilute mixture of nanotubes (0.23 mg/ml) and the quasi-isotropic-nematic transition was not observed immediately after shrinkage. It is evident from the photograph that the volume change ratio before/after shrinking is large (about eight times); the shrunken gels in FIGS. 7(a) and 7(b) also appear darker, apparently for the reason that the tube concentration is higher and the tubes absorb visible light.

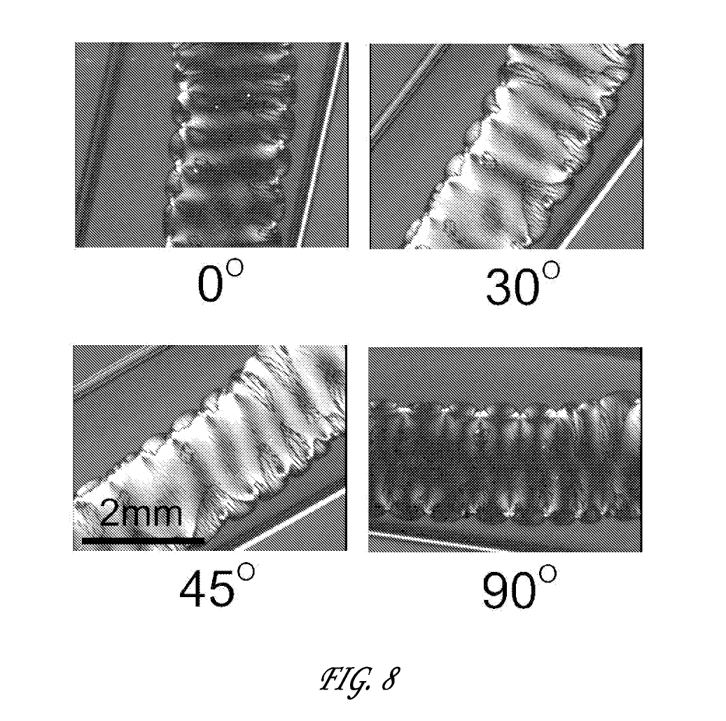

In FIG. 8 one of the concentrated samples is depicted as a function of angle. All the images were taken with fixed microscope bulb intensity and video gain and offset. This sample had an initial tube concentration of 0:78 mg/ml, and was allowed to sit for 4 days after shrinking. The gel exhibited a maximum birefringence when its edge was oriented 45 degrees with respect to the input polarizer pass axis. Liquid crystal like defects were observed near the edges with the sample; visible when the sample was in vertical (0 degree) or horizontal (90 degrees) orientations. Apparently there was greater tube alignment near the gel edges; the director tends to align near the walls, perhaps as a result of boundary effects. The darker regions in the center of the sample could indicate tube disorder or tube alignment in the z-direction. To distinguish these possibilities the sample was rotated between 10 to 60 degrees about the y-axis. Significant changes in the central birefringence profile were not observed, however. Most likely, the central dark regions were disordered.

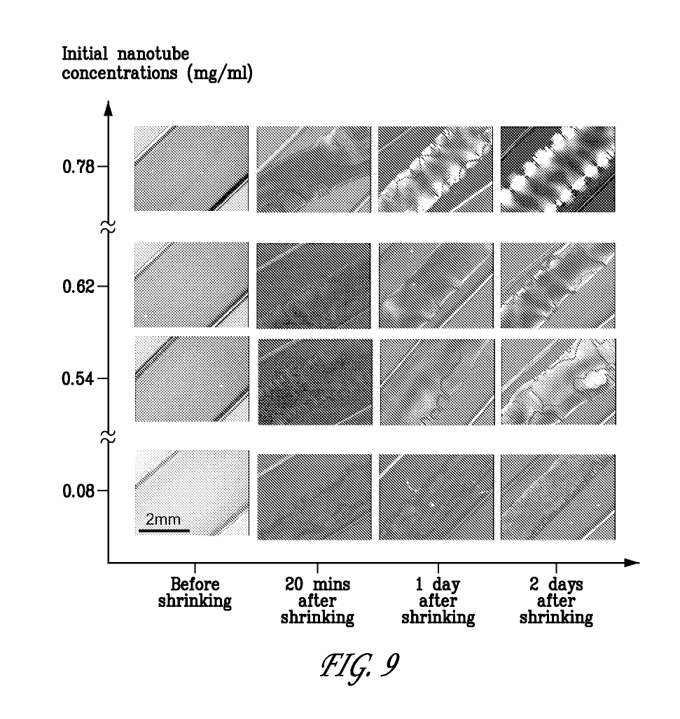

Features of our concentration- and time-dependent observations are summarized in FIG. 9. The bulb intensity and video gain/offset was the same as before. All of the samples were isotropic before shrinking; light transmission was zero. Birefringence was observed in samples that shrank. Twenty minutes after shrinking, the highest initial concentration (0.78 mg/ml) sample exhibited birefringence. As time passed though, the samples slowly evolved. Alignment clearly started at the edges of the sample and migrated inward. After one day the sample critical concentration for birefringence near the edge was approximately 0.54 mg/ml. After 2 days the critical concentration for birefringence had decreased further. The degree of nematic alignment in the samples was found to increase and the critical concentration for nematic alignment decrease, respectively, with increasing time after shrinking the gel.

These quasi “isotropic-nematic” transitions apparently differ from lyotropic transitions of suspended hard rods in some respects. The transition nanotube volume fractions were lower than expected based on nanotube behavior in water alone, suggesting the gel network plays a significant role in increasing the effective tube interaction, local concentration, or both.

In the above examples, the gel polymerization temperature was kept at 295 K. At this temperature, the gel network and the tube distribution within the gel was homogeneous. However, when polymerizing the NIPA monomer at a higher temperature (about 304 K), the nanotubes can micro-phase separate into regions of nanotube rich/gel poor regions and nanotube poor/gel rich regions. At high enough nanotube concentrations, the nanotubes in nanotube rich/gel poor region can align to become nematic. Such behavior is observed in other rod-like molecules (e.g., fd virus) in NIPA gel.

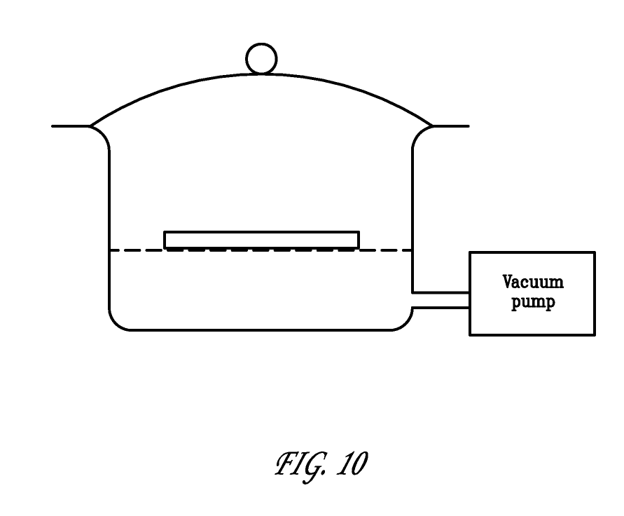

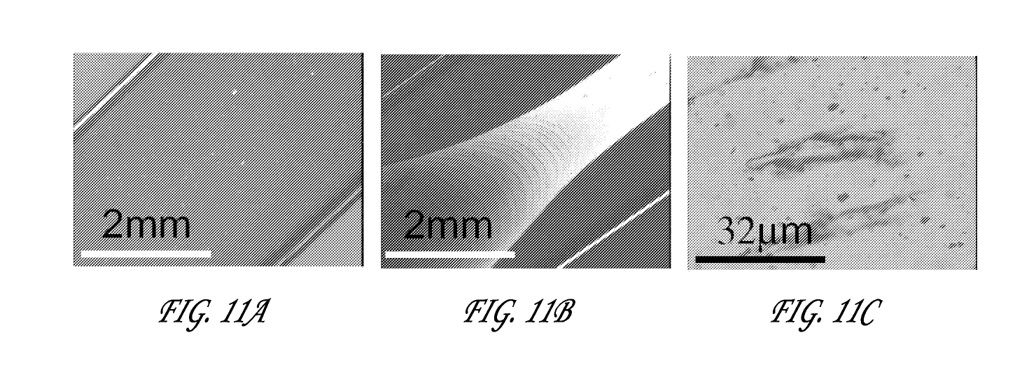

Method 2: Nanotube alignment via water extrusion from SWNTs-NIPA gels. To extrude water from SWNTs-NIPA gels, the capillary tubes containing the gels were placed inside a vacuum jar, which was slowly evacuated using a vacuum pump. The experimental setup is shown in FIG. 10. Initially, the nanotubes inside the gel were isotropic and the sample under cross-polarizers appeared dark as shown in FIG. 11(a). The slow vacuuming of the chamber caused water from the center of the samples to extrude (migrate) to the open ends of the capillary tubes and being evaporated off. The SWNTs-NIPA gel then started to shrink at the middle of the capillary tubes in width and thickness, as shown in FIG. 11(b). The flow out of water caused the nanotubes to align along the flow direction of water (the long axis of the capillary tubes) and the shrunk region became birefringent, as shown in FIG. 11(b). Eventually most of the water was extruded from the gel and the entire gel became birefringent. Typical sample dimensions before and after water extrusion were (length×width×thickness) about 4 cm×about 4 mm×about 0:2 mm, and about 2:8 cm×about 2 mm×about 0:1 mm, respectively. By varying the rate of water extrusion from the SWNTs-NIPA gels, or the initial nanotube concentrations in gel, or both, SWNTs were able to align or make small ropes as shown in FIG. 11(c).

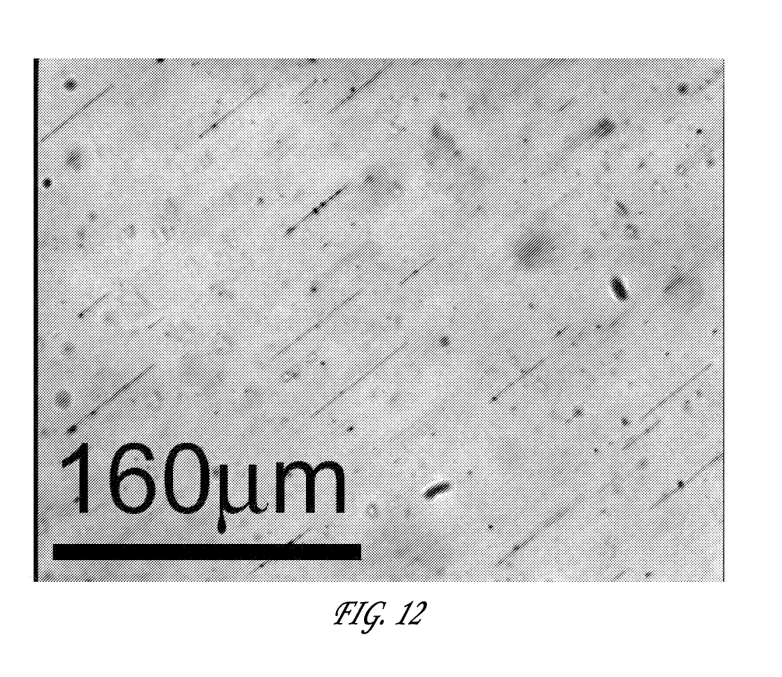

Method 3: Magnetic field induced alignment of nanotubes in NIPA gels. To align nanotubes inside NIPA gels, capillary tubes with SWNTs-NIPA gel were placed inside a super-conducting magnet while the gel was polymerizing. The applied magnetic field aligned the nanotubes along the magnetic field before the nanotubes got locked into position by the NIPA gels. The entire sample looked strongly birefringent under cross-polarizers indicating high degree of alignment of nanotubes in the gels. Surprisingly, nanotubes chained up to form “nanotube needles” under the magnetic field were also observed. FIG. 12 shows such an image for a sample with initial SWNTs concentration of 0:78 mg/ml. The formation of nanotube needles depended on the initial nanotube concentrations in gel, applied magnetic field strength, gel viscosity, and the presence or absence of iron in the carbon nanotubes. Carbon nanotubes containing iron readily aligned in a 9 T magnetic field. The gel viscosity was controlled by varying the NIPA monomer and the cross-linker concentrations. Lower gel viscosity allowed the nanotubes to move in the gel during the polymerization process to form long needles. Shrinking this gel did not destroy the nanotube needles, rather increased their number density and also slightly increased the birefringence of the sample.

Other gels and suspending materials. SWNTs were also dispersed in water, poly(methyl methacrylate) (PMMA) gel and poly(vinyl acetate) gel (PVA). Nanotube ropes with a length distribution of from 30 μm to 2 cm were obtained in water. In PMMA and PVA gel, SWNTs formed similar structures as those formed in NIPA gels.

Uses of Nematic Nanotube Gels. Nematic nanotube gels can be used to create high quality composites for various applications. Examples are provided below.

Polymer composites containing nematic nanotubes. Nanotubes are dispersed and aligned in various types of polymer gels. The alignment approach is very useful because aligned nanotubes and nanotube needles can be readily formed in polymeric gels according to the methods described herein. As aligned nanotubes increase the strength and thermal properties of composites, composites having aligned nanotube needles should also be capable of dissipating heat. Following the various orienting procedures described in these examples, polymer composites containing nematic nanotubes are prepared using an orienting field, such as stretching fibers and films of a rubbery polyester resin that contain SWNTs. In another example, polymeric composites of styrenic thermoplastics that contain nematic SWNT nanotubes are obtained by shearing styrenic thermoplastic fluids that containing nanotubes at an elevated temperature, which is followed by cooling upon cessation of the shearing. In another example, the nanotubes are oriented using a 1 T magnetic field in a polymeric liquid, such as rubbery PMMA at an elevated temperature, followed by cooling.

Curable resins having nematic nanotube gels. This example provides one solution to incorporating aligned nanotubes in a curable resins, such as epoxies, at high concentration. In this example, carbon nanotubes are dispersed in an epoxy and curing agent gel precursor as described above. Curing is carried out in the presence of an orienting field, such as a shear field arising from flow of the gel precursor dispersion in a microchannel device. In another example, the orienting field is a magnetic field and the general procedures described in Method 3, above, are used to orient the carbon nanotubes in the epoxy before the solid matrix completely hardens. Such resulting cured resins containing nematic nanotubes are useful in a variety of aerospace and semiconductor applications.

### Example: Copolymer Composed of Carbon Nanotubes

Traditional oxidization methods are used to introduce carboxylic acid group (—COOH) to both ends of single-wall carbon nanotubes. Through esterification or amidation of the ends' carboxylic groups, isolated SWNTs are linked with intermediate polymer chains to provide a polymer that is schematically illustrated as in FIG. 13(a)—number of linked units will vary considerably. This process can be used to prepare the copolymer containing nanotube in two steps:

First, one oxidizes and cuts commercial CNT and chemically modifies the two ends of CNTs into carboxylic groups. One prepares 0.2 wt % CNT in H₂SO₄/HNO₃ (3:1) and sonicates it for 24 hour at room temperature. Dilute it 5 times with DIUF water, and filter through 0.2 μm filter. CNTs are deposited on the filter paper. Put the filter paper with CNT on top of it in DIUF water and sonicate it. The filter paper is removed and the filtered CNT is kept in solution. Centrifuge the solution at 11,500 rpm for 30 minutes and redisperse it into DIUF water at 1 wt % concentration. Prepare 4:1 concentrated H₂SO₄: 30% H₂O₂ solution. Mix concentrated CNT solution with oxidization mixture and heateit with stirring at 90° C. overnight. Dilute the solution 5 times and centrifuge it at 11,500 rpm for 30 minutes. Decant the supernatant and dry the powder. The modified CNTs will have one or more carboxylic acid groups covalently bonded at both ends of the nanotubes as illustrated in FIG. 13(b).

Second, add functionalized CNT powder to monomer solution and sonicate it. After CNT is dispersed well, add initiator to the solution to start the polymerization. Alternatively, one can prepare functionalized dispersion and add monomer to the dispersion, and then add initiator to start the polymerization. Cross-linker can be used if building a cross-linked polymer network out of carbon nanotubes is desired. In order to set up a covalent bond between functionalized CNT and the monomer, the monomer contains an amine group (—NH₂) or alcohol group (—OH).

Example. Poly(PEO-co-CNT). This example provides a copolymer composed of alternating units of polyethylene oxide (“PEO”) and SWNTs. A similar approach can be used to prepare copolymers of any water soluble polymer (e.g., DNA, N-isopropyl acrylamide, and the like) and SWNTs.

### CNT Functionalization: Amidation of CNT and NH₂—PEG-NH₂

| Reactants | Amount | |
|---|---|---|
| 1 wt % oxidized CNT solution | 300 | μL |
| NH₂-PEG-NH₂ | 3.4 | mg |
| EDAC (1-ethyl-3-(30dimethylaminopropyl)- | 2 | μM |
| carbodiimide hydrochloride | | |

Procedure: Prepare 1 cc 500 mM MES buffer, pH at 6.1. Dilute it to 2.7 cc 30 mM buffer by adding DIUF water. Add 0.3 cc 1 wt % oxidized CNT solution to the buffer. Add 3.4 mg NH₂—PEG-NH₂ to the buffer. Sonicate the dispersion for half hour. Prepare EDAC stock solution immediately before use (52 μmmol/mL). Add EDAC solution to the buffer and mix the solution rapidly by syringing repeatedly with the pipette. Sonicate the solution for 1 hour.

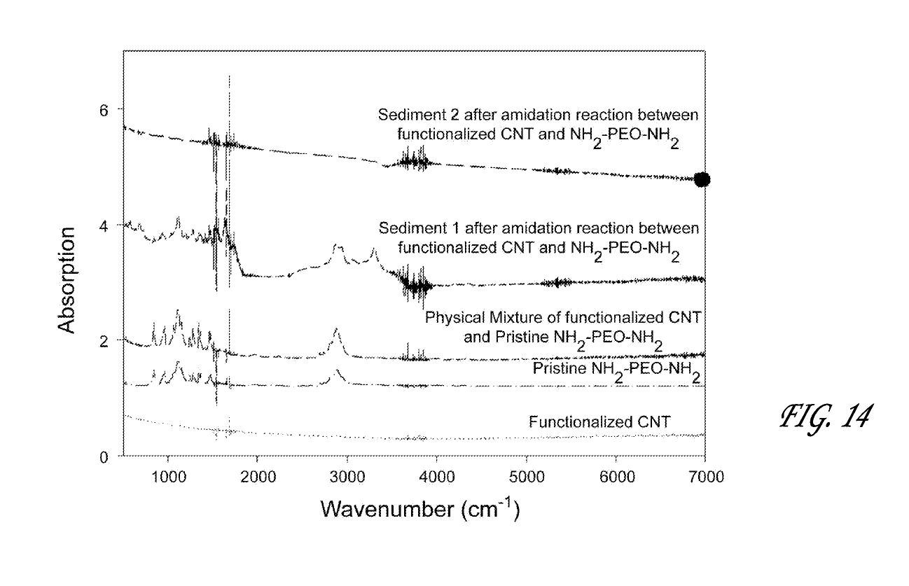

Example. Centrifuged the CNT+NH₂—PEG-NH₂ at 11,500 g for 2 hours to sediment CNTs that reacted with NH₂—PEG-NH₂. Call this sedimentation I. Then sedimented the remaining CNTs in suspension by centrifuging the suspension at 355,000 g for 4 hours. Call this Sedimentation 2. FIG. 14 shows FTIR absorption on pristine CNT, pristine NH₂—PEG-NH₂, physical mixture of CNT & NH₂—PEG-NH₂, Sedimentation 1 (first sedimentation by centrifugation after chemical reaction of CNT and NH₂—PEG-NH₂) and Sedimentation 2 (second sedimentation by centrifugation after chemical reaction of CNT & NH₂—PEG-NH₂. The FTIR absorption spectrum of physical mixture of CNT & NH₂—PEG-NH₂ is the overlap of individual spectrums of pristine CNT and pristine NH₂—PEG-NH₂. However, the absorption spectrum of Sedimentation 1 is more than the simple addition of the individual spectrums of pristine CNT and pristine NH₂—PEG-NH₂, which indicates that some new chemical structure was formed. The FTIR spectra for Sedimentation 2 look quite similar to the spectrum of pristine CNT. Accordingly, Sedimentation 2 appears to contain primarily un-reacted CNT.

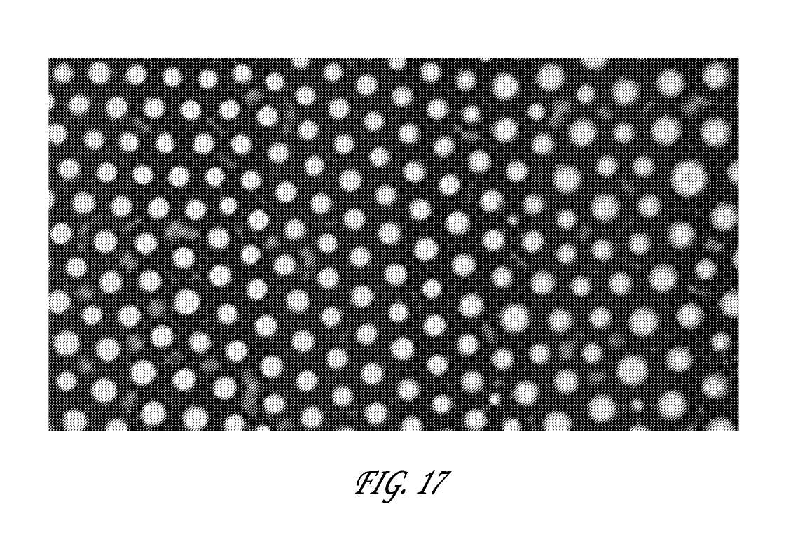

Example: Polymer particles coated with carbon nanotubes. A microfluidic T-channel as illustrated in FIG. 15 was used to synthesize aqueous dispersions of positively charged polystyrene sulfonate particles coated with negatively charged NaDDBS dispersed carbon nanotubes. A PTFE channel mold was constructed, and then a PDMS imprint was made using the PTFE channel mold. Styrene sulfonate (SS) monomer was commercially obtained from Aldrich. A CNT+NaDDBS+water dispersion had 0.1 wt % CNTs, 1 wt % NaDDBS and balance water. In preparing PSS emulsions in CNT-NaDDBS-water, SS was fluidically transported through fluid conduit (channel) 1512 using a syringe pump with a flow rate of about 100-200 microliters/hour. The CNT+NaDDBs+water dispersion was fluidically transported through fluid conduit (channel) 1502 at a flow rate of about 100-200 microliters/hour. (Note: FIG. 15 is labeled for making CNT+NaDDBS+water emulsions in organic solution, but can be adapted, as in this example, to make an organic phase dispersed in an aqueous dispersed carbon nanotube phase. Accordingly, the type of liquids flowing through conduits 1502 and 1512 can be hydrophobic, hydrophilic, or both.) In this example, PSS—CNT composites are made by preparing SS emulsion particles (the organic phase) in CNT-NaDDBS-water (the aqueous phase) and polymerizing the SS monomers. A photomicrograph of the resulting PSS emulsion polymer particles coated with carbon nanotubes (average particle size is about 30 microns) is provided in FIG. 17.

Example. Supercapacitor. Water based dispersions of PEDT/PSS (Mw 65000/14000) commercially available from Bayer, Pittsburgh, Pa. can be used to form composite materials. CNT+NaDDBS aqueous dispersions of 0.8 wt % concentration are blended with PEDT-PSS and EDT monomers in water. At this concentration, CNTs will form a percolating NT network. PSS of PEDT-PSS will be closer to the NT network. The EDT is then polymerized to create conducting but electrically separated network from the conducting CNT network. This will then result in a supercapacitor.

Example. Electrically Conductive and Strong Composites. PSS-PEDT-PSS containing NH2 groups at both ends are covalently bonded to CNT segments having functionalized carboxylic acid groups at both ends. An amidine reaction between the PSS-PEDT-PSS and CNT segments gives rise to electrically conducting (due to PEDT) and strong (due to CNTs) polymer-CNT composites.

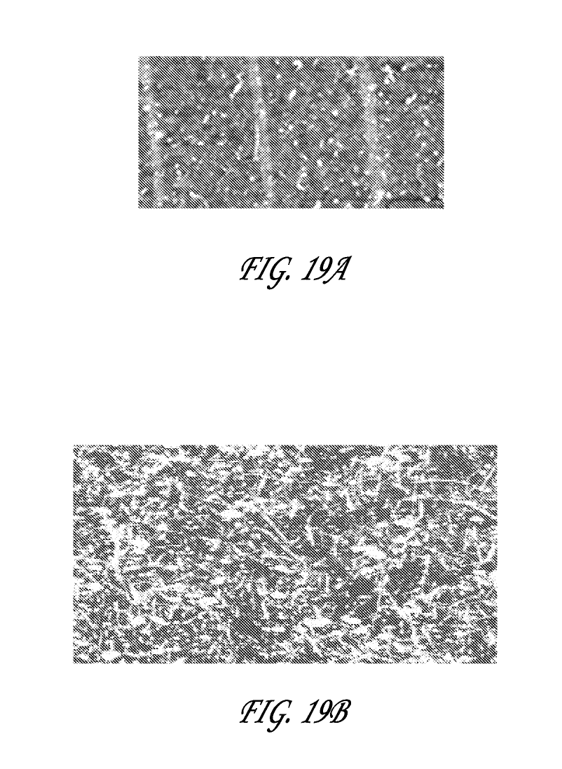

Example. Controlled Deposition of Carbon Nanotubes on Silicon Wafer. The process generally described in FIG. 18 hereinabove was used to controllably deposit carbon nanotubes (CNT) on a silicon wafer. This process was used to deposit isolated dispersed carbon nanotubes on top of a silicon wafer within a controlled area, whose dimension can be made as small as 100 nm. NaDDBS was used to disperse isolated carbon nanotubes in aqueous phase. Without being bound by any particular theory of operation, it is believed that the surfactant covered the nanotube exterior surface. Since each NaDDBS molecule has a negative charged head group (—SO₃⁻), the NaDDBS dispersed CNT are highly negatively charged. A self-assembled molecular monolayer of APTS (3-Aminopropyltriethoxysilane) is chemically linked on top of an oxidized layer that surmounts the silicon wafer. The monolayer is highly positively charged, which has a strong ability to attract negatively charged CNT. Standard e-beam lithography was used to fabricate as small as 100 nm channels inside the PMMA layer on top of a silicon wafer with around 300 nm-400 nm oxidized top layer. The e-beam resist was 4% 495K molecular weight PMMA in chlorobenzene. The resist was spin coated on top of a silicon wafer at 3000 rpm. The resulting resist thickness was between 300 nm and 400 nm. The resist was baked on a hotplate for 30 minutes at 190° C. E-beam lithography was used to etch channels inside of the PMMA layer. 30 μA e-beam current, 4 pix step size and 400 μA/cm² dose was used during the e-beam lithography. An AFM image of the resulting patterned substrate 1900 is shown in FIG. 19(a) that shows channels 1904 located between non-channel areas 1902. Channel size ˜200 nm wide.

Vapor deposition was used to graft an APTS self-assembled molecular monolayer on top of an oxidized silicon wafer. An 8 ounce glass bottle and a 2 cm height glass ring are used. The oxidized silicon wafer is placed on top of the glass ring facing up. 2 cc APTS are added to the glass bottle. Dry N₂ is blown into the glass bottle for a half minute. The glass bottle is sealed with parafilm and placed on a hot plate at 80° C. for 10 minutes. The APTS-treated substrate is removed and sonicated in DIUF water for half hour. The resulting surface is contacted with 0.01 wt % NaDDBS dispersed CNT solution overnight. The AFM picture in FIG. 19(b) shows full coverage of CNT tubes 1906 on top of the wafer surface.

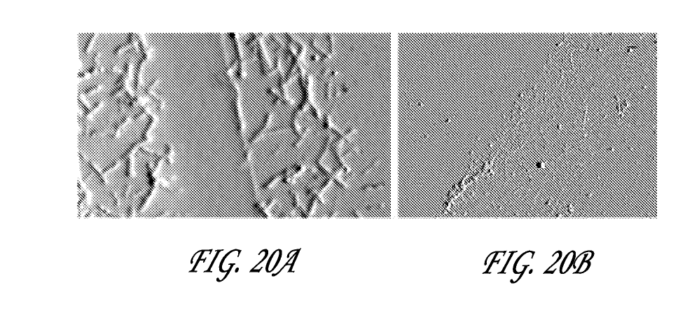

Example. Plasma oxidization was used to clean up a channel surface on an E-beam patterned substrate. The operation power was 100 w, and the duration time was 10 seconds. Vapor deposition was used to graft APTS self-assembled molecular monolayer on top of the exposed silicon oxidized area inside the patterned channel. The APTS monolayer was then exposed to fuming HCl to convert APTS' NH₂ groups to NH₃⁺ groups. The sample was contacted with 0.1 wt % NaDDBS dispersed CNT solution for 24 hours. Finally, the PMMA layer was lifted off with acetone. Alternatively, the PMMA layer can be lifted off before contacting the wafer with NaDDBS dispersed CNT solution. The substrate was immersed in acetone for about 1 minute and then immediately took out and rinsed with plenty of DIUF water. It was blown dry with dry air and baked in an oven for at least four hours at 200° C. to burn off NaDDBS. The surface of the substrate comprising the controllably deposited nanotubes was characterized using tapping mode AFM, as shown in FIGS. 20(a) and (b). In FIG. 20(a), nanotubes 2002 are shown controllably deposited in channel 2004, having channel width about 2.5 microns wide. The surface 2008 of the substrate has regions having deposited nanotubes 2004 and regions devoid of nanotubes 2006. FIG. 20(b) is of a different geometry, the nanotube deposited channel width 2004 is in the range of about 10 to 15 microns wide.

A major obstacle in the industry applications of CNT is to manipulate and assemble CNTs in a desired way for consequent processes. For example, electronic circuits require accurate locations of CNT in order to make integrated circuits for computers. Control deposition offers a convenient solution for this. This technology can be easily applied to mass production of exactly depositing isolated CNT inside target areas on top of a silicon wafer. The procedures provided herein are capable to put thousands of isolated SWNT inside a single channel with little effort. The deposited CNT can be consequently used as components of single molecular electronic circuits. These circuits can be used as FET for the application of logic circuits, or single electron detectors, or even as recently discovered, they can be used as molecular photon-emitters. Additionally, the deposited CNT can be used as components of gas sensors and biosensors. When exposed to O₂, NO₂ or NH₃, the electrical conductance or dielectric constant of semiconducting SWNT changes. When CNTs are derivatized with functional groups, such as bifunctional molecule, for example, 1-pyrenebutanoic acid succinimide ester, CNTs can immobilize biomolecules. Depositing CNT in target areas offers a great tool to materialize these possibilities into commercial products. Controlled deposition of nanotubes enables the fabrication and manipulation of nanotube-based circuits and sensors. For example, putting isolated SWNT into target areas makes it plausible to fabricate integrated circuits, and sparsely orderly distributed SWNT circuits are ideal components for biosensors.

Biosensors are also prepared by chemically grafting amine group to NaDDBS to replace the sulfonate group. The carboxylic group from the surfactant can form a peptide bond with amine group found in various bio-molecules (e.g, DNA, protein, and the like):

R—COOH+R′—NH₂→R—CONH—R′+H₂O

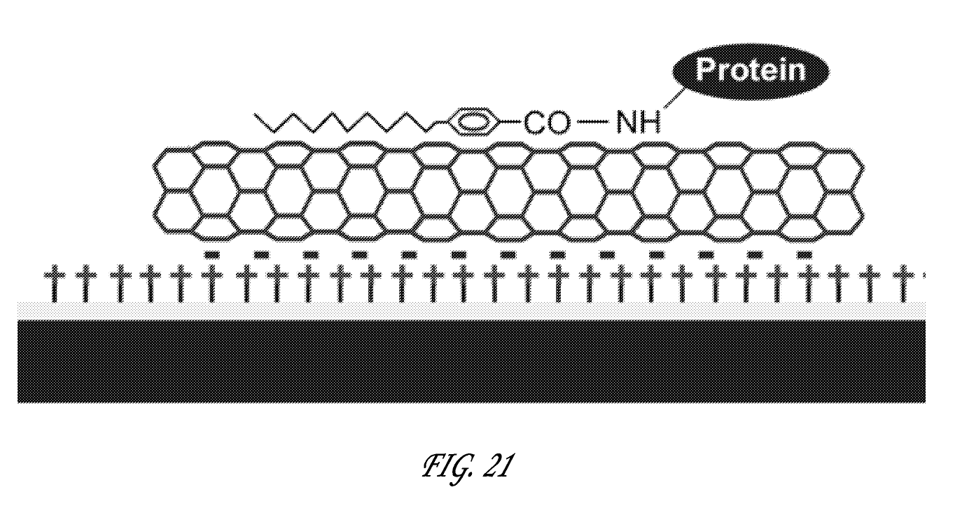

The combined protein or DNA molecule will change the CNT's electron distribution. Consequently the electric response to the external voltage will be different for CNT with and without binding bio-molecules. An example of a biosensor element 2100 is provided in FIG. 21, which shows a substrate 2102 (silicon wafer) having an oxidized layer 2106 (silicon dioxide) and positively charged linker molecules 2106 bound to the oxidized layer 2106. Carbon nanotube 2110 has negative charges 2108 that keep it bound to the positively charged linker molecules 2106. A surfactant molecule 2112 having a carboxylic acid functional group is shown forming an amide linkage with an amine functional group of a protein molecule 2114.

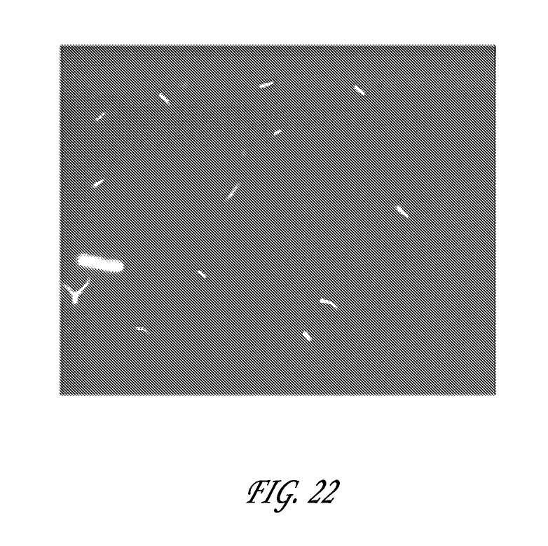

Example. Operation of a CNT-Based Sensor Element. A sensor element was prepared as follows. Carboxylic group grafted surfactant aqueous dispersed CNTs and sulfonic group grafted surfactant aqueous dispersed CNT were deposited onto two APTS treated glass slides. Amine group grafted dye solutions were applied to both glass slides and imaged using fluorescence optical microscopy to ascertain that the dye was grafted to the CNTs. Only the carboxylic group grafted surfactant dispersed CNT sample showed strong fluorescence while the sulfonic group grafted sample was totally dark. The fluorescence optical microscopy of the carboxylic group grafted sample in FIG. 22 shows fluorescent-dyed labeled carbon nanotubes adsorbed onto a substrate.

Example. Molecular Photon Emitters Composed of Controllably-deposited CNTs on Substrates. The general procedure described by Misewich, in Science, vol. 300. p. 783 (2003) for preparing CNT-based photon emitters is referenced herein. Charged CNTs are controllably deposited on patterned charged areas of a p-silicon substrate with 150 nm thick SiO2 layer according to the methods described hererinabove. Source and drain contacts are fabricated using standard lithographic techniques. The source and drain are 50 nm thick titanium film. The whole device (CNT, source and drain) is then coated with 10 nm thick SiO₂ layer. When the CNTs exhibit ambipolar behavior, a simultaneous injection of electron and hole into the device causes CNT to emit infrared (IR) wavelength photons. The simultaneous injection of electron and hole is achieved by biasing the device with a gate potential that is between the potential of the source and the gain of the device. For example, the source is grounded, the gate is +5V and the drain is +10 V. The difference of the potentials of the source and the drain with respect to that of the gate is 5V. Because the sign of the gate field is opposite at each end, the gate field at the source draws electrons into the device, and the gate field at the drain draws in holes into the device. The combination of electrons and holes results in light emission.

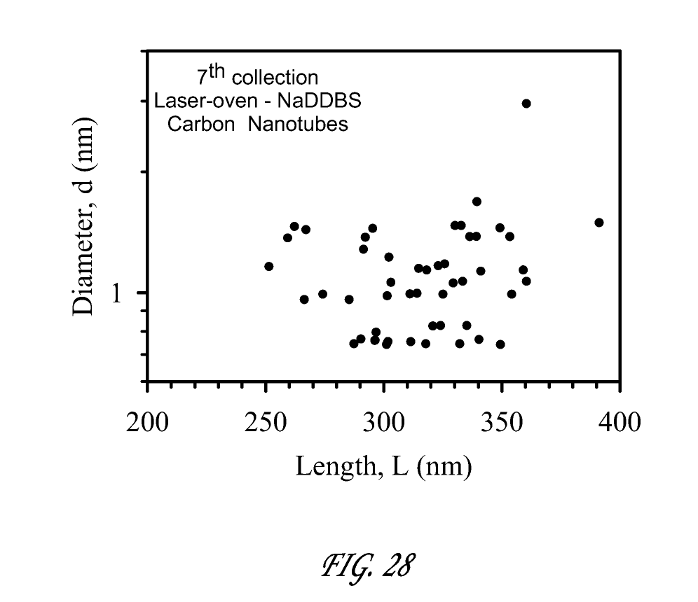

Example. Carbon Nanotube Length Separation. Firstly, we prepared a 0.1 wt % NaDDBS dispersed SWNT dispersion. We used mortar to grind high concentration pristine CNT solution. A small amount of NaDDBS was added to the solution to help to disperse CNT. After grinding, we added more DIUF water and NaDDBS to form 0.1 wt % SWNT dispersions. The mass ratio between NaDDBS and CNT was 10. We tip sonicated the dispersion for 10 minutes at 8 watts. Then we bath sonicated the dispersion at 12 watts, 55 kHz for 24 hours. Secondly, we used gel-exclusion chromatography to separate the isolated SWNT dispersion. The gel we used was Sephacryl™ S-1000 superfine gel (from Amersham Biosciences). The gel's cutoff pore size is ˜300 nm. In order to separate CNT with length greater than 300 nm, we added MMA monomer to the gel beads and polymerize it to form cross-link among the gel beads. Thus micron size pores were produced. The gel chromatography tube was Econo-Column chromatography column from Bio-Rad. We used standard gel-exclusion chromatography procedure to fractionate our dispersion: we run the dispersion through the gel under gravity and collected the elutes in time sequence. The CNT diameter versus length results of a seventh collection of laser-oven carbon nanotubes separated according to this method is provided in FIG. 28. The average length of the nanotubes is 316 nm+/−30 nm.

Example: Purified SWNTs for use in electronic devices. As-grown HiPCO material was purified by heating in wet air in the presence of H₂O₂, gentle acid treatment, magnetic fractionation (Islam, et al., Phys. Rev. Lett. 93, —(2004)) and vacuum annealing. The dominant impurities in as-grown HiPCO were catalyst particles and non-SWNT carbon phases. Thermogravimetric analysis and wide-angle X-ray scattering measurements indicated impurity content was more than 50 wt % in as-grown HiPCO and less than 5 wt % after purification. Based on this measured impurity content and the measured sample mass after purification, the purification process recovered close to 90% of the SWNT content of the HiPCO. Further details of the SWNT purification process are provided below.

Wet Air Burn:
1. Impurity carbon phases (amorphous carbon, fullerenes, etc.) are removed by heating HiPCO material in air in the presence of H₂O₂ for 3-6 hours.

Acid Treatment:
2. Oxidized SWNT material is refluxed with 2-3 M HNO₃ for 20 minutes, neutralized with NaOH, and then washed with deionized water.
3. Material is refluxed with H₂O₂ for 10 minutes.
4. Steps 2, 3 are repeated 2-3 times.

Annealing
5. Material is annealed in vacuum at 1150 C for 2-3 hours.

Magnetic Fractionation
6. SWNT material is dispersed in NaDDBS surfactant solution as detailed in Ref 11. The material is flowed over a magnetic field gradient (˜0.08 T/cm). Magnetic impurities feel a force due to the field gradient and are removed from the main flow of material.

Thermogravimetric analysis and wide-angle X-ray scattering measurements indicate impurity content is more than 50 wt % in as-grown HiPCO and less than 5 wt % after purification. Based on this measured impurity content and the measured sample mass after purification, the purification process recovers close to 90 wt % of the SWNT content of the HiPCO.

The material to be tested (either raw or purified HiPCO) was dispersed in water using sodium dodecyl benzene sulfonate (NaDDBS) and deposited onto degenerately doped oxidized (400 nm SiO₂) silicon wafers. Prior to deposition, the SiO₂ surface is functionalized with a 3-aminopropyl triethoxysilane (APTS) monolayer, and SWNTs were deposited by briefly dipping the chip in the SWNT-NaDDBS suspension. The sample was rinsed in deionized water, blown dry, and heated in air at 200° C. for 12 hours. This last step removed a large fraction of the residual surfactant as evidenced by a systematic ˜2 nm decrease of the nanotube diameter as measured by atomic force microscopy (AFM). This treatment also vaporized the APTS monolayer from the bulk of the silicon substrate.

FIG. 24(a) is an AFM image of individual SWNTs and small nanotube bundles (collectively 2404) on functionalized substrate surface 2402 after deposition from solution and surfactant removal. Cr/Au source and drain electrodes separated by 400 nm were fabricated with electron beam lithography without alignment followed by thermal evaporation and liftoff (FIG. 24(b): electrodes 2406, space 2408 between the electrodes 2406, nanotubes 2404 adjacent to both the electrodes and the space between the electrodes; FIG. 24(c): source electrode 2410, drain electrode 2412, gap or space 2414 with a SWNT 2416 spanning the source and drain electrodes—the image of the SWNT is highlighted using a white line.) The electrode density was chosen so ˜50% of the electrode pairs conduct, typically contacting one SWNT or one small bundle. A degenerately doped Si was used as a back gate electrode in a field effect transistor (FET) geometry.

The source-drain current I was measured in ambient conditions for different values of the bias voltage V_{b} and gate voltage V_{g}. The behavior of the I-V_{g} curve at low voltage bias (typical V_{b}=10-100 mV) was used to categorize each sample as “metallic” (M), “semiconducting” (SC), or “hybrid” (H). “Metallic” samples had a relatively low source-drain resistance and I shows little or no gate response; we conclude these samples consisted of a single metallic SWNT or a bundle where only metallic SWNTs were contacted. “Semiconducting” samples exhibit a high ON/OFF ratio, with very large resistance in the OFF state. Without being bound by any particular theory of operation, we presume conduction occurs through a single semiconducting SWNT or a bundle where current is carried by semiconducting nanotubes. “Hybrid” samples exhibit a small ON/OFF ratio of roughly 2-4. We attribute this behavior to conduction by metallic and semiconducting SWNTs in parallel. FIG. 24(d) shows examples of these three observed behaviors of SWNTs: metallic, hybrid, and semiconducting.

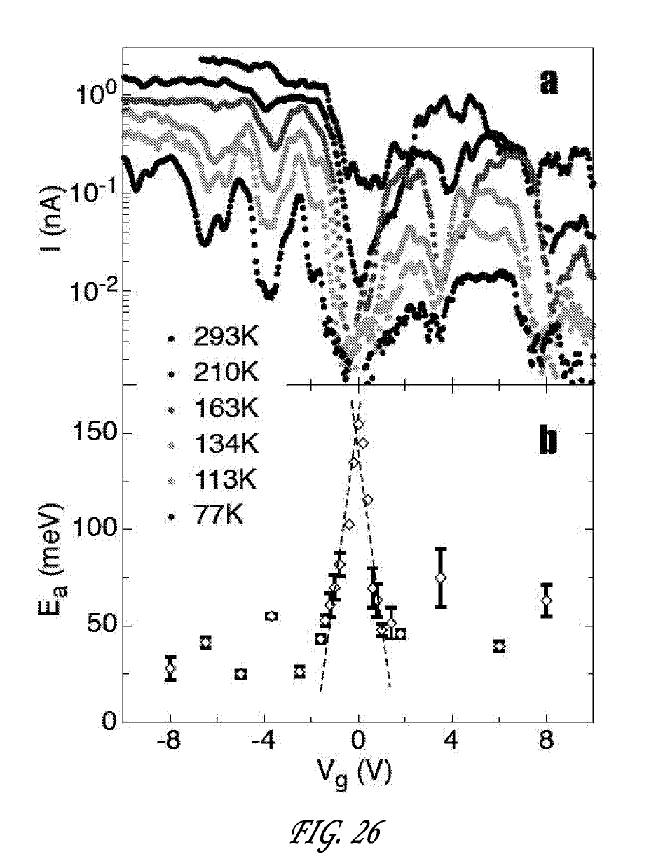

The quality of the purification process was tested by comparing circuits made using as-grown and purified samples from the same HiPCO batch. The fraction of conducting samples was ˜25% ( 4/16 for raw and 7/30 for purified material) for both fabrication runs. Quoted resistance values for SC samples are for the “ON” state (V_{g}=−10 V). M and SC circuits made from raw HiPCO had source-drain resistances near 1 GΩ, while for purified material we measured a median resistance of 4 MΩ for M/H samples; no SC samples were observed in this first trial. Purification thus leads to a decrease in sample resistance by a factor of more than 200. The electrical transport properties of 29 additional samples made from purified material were then measured and classified. Twenty-two samples were M/H with a median resistance of 500 KO. Seven samples were SC with a median resistance of 10 MO. These should be compared with typical resistances of 15 kΩ and 100 kΩ for M and SC circuits made in our lab with CVD-grown SWNTs. The typical ON/OFF ratio of SC devices was 300, with the highest exceeding 5000. Six of the SC samples exhibited p-type gate behavior similar to FETs made from CVD-grown SWNTs; one SC device had an ambipolar gate response, with both hole and electron conduction (FIG. 26(a)). Table 1 below provides a complete listing of observed sample resistances.

TABLE 1
Observed Resistance Values

| | Run 1 | Run 2 | Run 3 |
|---|---|---|---|
| | Raw HiPCO | Purified HiPCO | Purified HiPCO |
| Device Yield | 4/16 | 7/30 | 29/98 |
| SC Devices | 2 | 1 | 7 |
| Resistances (MΩ) | 700, 2000 | 140 | 1.4, 10, 10, 10, |
| | | | 20, 250, 1400 |
| M/H Devices | 2 | 6 | 22 |
| Resistances (MΩ) | 570, 1300 | 0.5, 0.5, 4, | Median = 1 |
| | | 8, 10, 2000 | Mean = 0.5 |

Run 1 and Run2 use material from the same batch of HiPCO.
Measured resistance values (MΩ) for Run 3, M/H devices are: 0.1, 0.13, 0.15 (x2), 0.17 (x3), 0.25 (x2), 0.3, 0.5 (x2), 0.55, 0.6, 1, 1.4, 2, 3 (x2), 5.

The observed fraction of SC samples (24%) is consistent with HiPCO material having random chirality (i.e., ⅔ semiconducting SWNTs and ⅓ metallic). If we assume small SWNT bundles (2-4 nm diameter measured by AFM as seen in FIG. 1a) show SC behavior only if all of the 3-4 SWNTs on the bundle exterior contacted by the electrodes are semiconducting (Radosavljevic, et al., Phys. Rev B, Rapid Communications 64, 241307 (2001)) then we expect 20-30% of samples to be SC, in satisfactory agreement with the data. The measurement of additional single-tube circuits would more precisely quantify the distribution of metallic and semiconducting SWNTs produced by the HiPCO process.

Three sources increase the resistance in SWNT circuits above the quantum limit of h/4e²≈6.4 kΩ. Contaminants on the SWNT sidewall increase contact resistance by acting as tunnel barriers at the electrodes or causing poor wetting of the electrode metallization. Schottky barriers form at the contacts to semiconducting (but not metallic) SWNTs, with minimum (tunnel) resistance near 100 kΩ. (Freitag, M., et al., Appl. Phys. Lett. 79, 3326-3328 (2001); Appenzeller, J., et al., Phys. Rev. Lett. 89, 126801 (2002)). Finally, electron backscattering along the length of the SWNT contributes to resistance. The carrier mean free path for HiPCO is unknown but it can be several micrometers for clean metallic and semiconducting (Durkop, T., et al., Nano Lett. 4, 35-39 (2004)) SWNTs grown by CVD.

The high, nearly equal, resistances observed for M and SC devices from as-grown HiPCO indicate that in these samples sidewall contamination is the dominant source of resistance. The new purification process reduces the resistance of both types of samples by a factor of several hundred or more. Despite this improvement, devices from purified HiPCO have resistances significantly larger than those produced from CVD SWNTs.

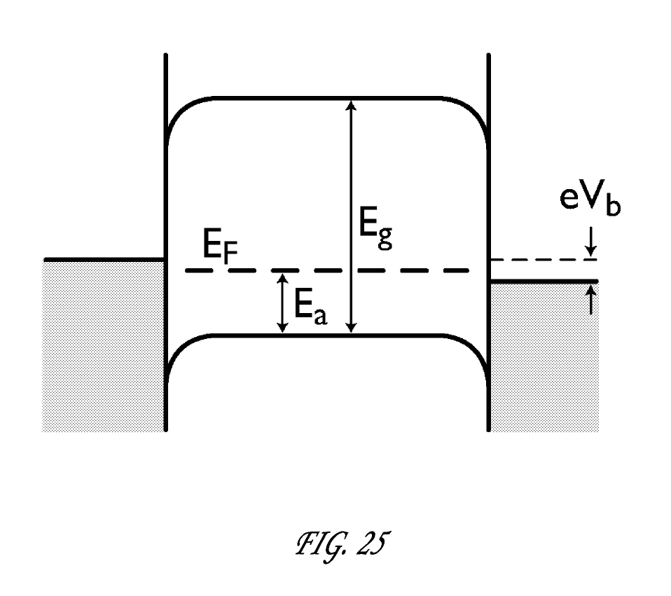

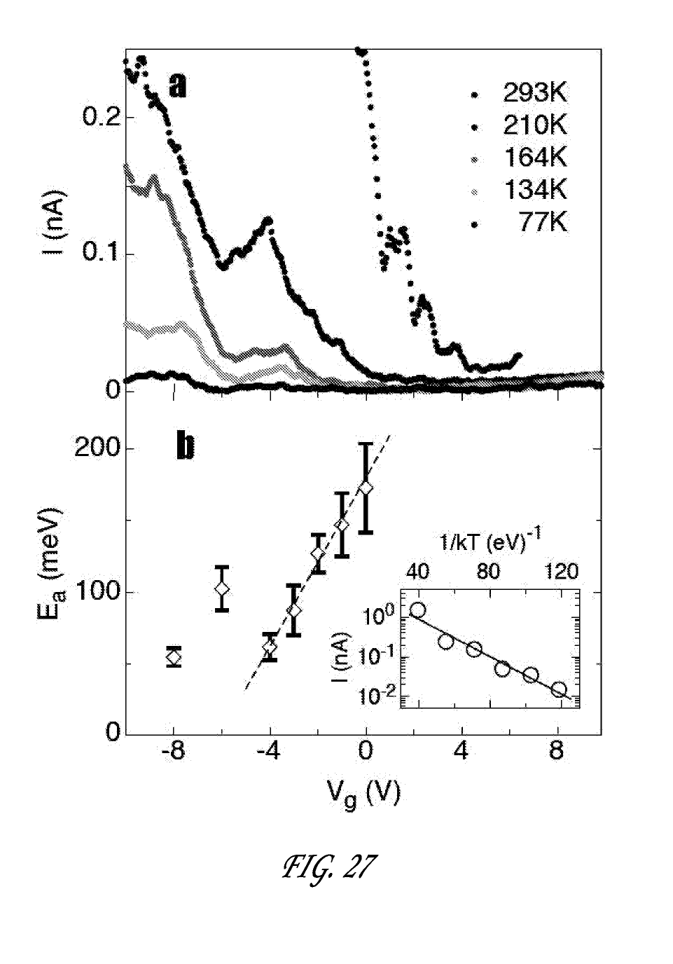

Temperature dependent measurements of SC circuits made from purified material are consistent with thermally activated transport, with an activation energy Eₐ that varies with gate voltage (i.e., I(V_{g},T)∝e^{-E}^{ₐ}^{(V}^{_{g}}^{)/k}^{_{B}}^{T}, where k_{B} is Boltzmann's constant). Without being bound by any particular theory of operation, this effect is understood as follows. Schottky barriers form at the contacts to nanotube FETs (FIG. 25). Energy band pinning in such devices is commonly asymmetric, so holes conduct more readily than electrons. Electron conduction is typically still measurable in large-diameter (small energy bandgap) SWNTs, leading to ambipolar I(V_{g}) characteristics (FIG. 26). In contrast, small diameter (large bandgap) SWNTs typically show p-type conduction, with electron conduction suppressed below measurement sensitivity (FIG. 27). As described below, the data agree with a model where the Schottky barrier acts as a tunnel barrier with different, temperature-independent transparencies for the two carrier types. When V_{g} is set so the Fermi energy E_{F} lies in the band gap, transport occurs with an activation energy given by Eₐ=|E_{F}−E_{band}|, where E_{band} is the energy band edge (valence or conduction) closest to E_{F}; the activation energy is therefore expected to vary linearly with V_{g}, reaching a maximum of half the energy band gap when E_{F} is situated mid-gap.

We observe this behavior for the ambipolar sample Device I. FIG. 26 shows the temperature dependence of I(V_{g}) for this sample, the 4 nm diameter bundle imaged in FIG. 24(c). We used Scanning Impedance Microscopy to verify that this structure was the only current path connecting source and drain contacts. For fixed V_{g}, the source-drain current data show the expected thermally-activated dependence. We use an Arrhenius plot to extract an activation energy Eₐ (FIG. 4b, inset), which is plotted as a function of V_{g} in FIG. 26(b).

The linear regions in FIG. 26(b) (−2 V<V_{g}<2 V) occur when E_{F} is situated in the band gap of the semiconducting SWNT. At V_{g}=0, Eₐ reaches a maximum of about 150 meV; the energy gap of this SWNT is therefore 300 meV, corresponding to a nanotube diameter near 2 nm that is compatible with AFM images of the structure (FIG. 24). From a linear fit to Eₐ in the gap region, the ratio of gate capacitance to total capacitance, or “lever arm”, is found to be cc 0.08, similar to the value of 0.1 found for CVD-grown samples with the same device geometry. The activation energy oscillates for −8 V<V_{g}<−2 V as does I(V_{g}). We attribute these oscillations to single electron charging and note that a maximum (minimum) in the activation energy near V_{g}=−6 V (V_{g}=−8 V, −4 V) corresponds to a minimum (maximum) in I(V_{g}), as expected for the charging regime.

FIG. 27 shows I(V_{g}) data as a function of temperature for Device II, a p-type FET. Again we observe that Eₐ increases linearly with gate voltage in the range −4 V<V_{g}<0 V. We can not determine Eₐ for positive V_{g} because the current at low temperature is below measurement sensitivity, but the data indicate an energy gap greater than 400 meV and a lever arm α≈0.03. Similar to Device I, I(V_{g}) and Eₐ(V_{g}) exhibit oscillations that are attributed to Coulomb effects.

SWNT nanoeletronic devices have been fabricated from bulk HiPCO-grown material. Devices fabricated from raw HiPCO have very high resistance; careful purification is used to remove impurities that would otherwise degrade the device characteristics. After purification, resuspension, deposition, and surfactant removal, SWNTs retain the unique electronic properties that make them useful in nanoelectronic devices. The energy gap of individual semiconducting nanotubes can be quantitatively inferred from measurements of device current as a function of temperature and gate voltage.

What is claimed:

1. A copolymer, comprising:
a plurality of end-linked single-wall carbon nanotubes.

2. The copolymer of claim 1, wherein:
said single-wall carbon nanotubes comprise at least one open end.

3. The copolymer of claim 1, wherein said single-wall carbon nanotubes comprise two open ends.

4. A compound, comprising:
a carbon nanotube comprising two open ends and at least one functional group bonded at each of said open ends.

5. The compound of claim 4, wherein said at least one functional group is capable of step-growth polymerization, chain-growth polymerization, or both.

6. The compound of claim 4, wherein said carbon nanotube is a single-wall carbon nanotube.

7. The compound of claim 4, wherein said functional group comprises a carboxylic acid group, an alcohol group, an amine group, an ethylenically unsaturated group, a ring-opening group, or any combination thereof.

8. A method, comprising:
opening both ends of a carbon nanotube;
providing at least one covalently-bound functional group to each of said ends; and
covalently bonding at least one monomeric compound to said at least one covalently-bound functional group.

9. The method of claim 8, further comprising dispersing said carbon nanotube in a fluid medium.

10. A composition, comprising the copolymer of claim 1.

11. A composition, comprising the compound of claim 4.

12. A polymer, comprising:
a chain structure of a plurality of covalently-bonded open-ended carbon nanotubes.

13. A method, comprising:
providing a T-channel microfluidic device, comprising:
a microchannel comprising an inlet, a junction and an exit;
a first fluid conduit capable of transporting a first fluid into the microchannel at said inlet;
a second fluid conduit, said second fluid conduit capable of transporting a second fluid into the microchannel at said junction;
fluidically transporting said first fluid from said first conduit into said microchannel;
fluidically transporting said second fluid from said second conduit into said microchannel, and;
forming a dispersed phase of said second fluid in a continuous phase of said first fluid in the microchannel, wherein said first fluid, said second fluid, or both, comprise an aqueous dispersion of carbon nanotubes.

14. The method of claim 13, wherein said dispersed phase, said continuous phase, or both, comprises a monomer.

15. The method of claim 14, further comprising the step of polymerizing said monomer.

16. A composition made according to the method of claim 15.

17. A method, comprising:
providing a patterned substrate comprising a polymer layer and exposed surface features;
bonding charged linker molecules, linker molecules capable of being charged, or both, to said exposed surface features;
removing said polymer layer;
optionally charging the linker molecules capable of being charged; and
bonding charged carbon nanotubes to the charged linker molecules, wherein the charge of the charged carbon nanotubes is opposite the charge of the charged linker molecules bonded to the exposed surface features.

18. The method of claim 17, wherein the charged linker molecules bonded to the exposed surface features are positively charged and the carbon nanotubes are negatively charged.

19. The method of claim 18, wherein the negatively charged carbon nanotubes comprise a surfactant comprising an aromatic group, an alkyl group having from about 4 to about 30 carbon atoms, and a negatively charged head group.

20. The method of claim 19, wherein said surfactant comprises hexylbenzene sulfonate, octylbenzene sulfonate, dodecylbenzene sulfonate, hexadecylbenzene sulfonate, or any combination thereof.

21. The method of claim 18, wherein said polymer layer comprises an acrylic polymer.

22. The method of claim 18, wherein said linker molecules capable of being positively charged comprise APTS.

23. The method of claim 18, further comprising the step of fluidically sealing a microfluidic assembly to said patterned substrate.

24. The method of claim 18, wherein said exposed surface features comprise a dimension smaller than about 500 nm.

25. The method of claim 18, wherein said exposed surface features comprise a dimension smaller than about 250 nm.

26. The method of claim 18, wherein said exposed surface features comprise a dimension smaller than about 100 nm.

27. The method of claim 18, wherein said exposed surface features comprise a trench.

28. The method of claim 18, wherein said positively charged linker molecules or linker molecules capable of being positively charged self assemble on said exposed surface features.

29. A substrate, comprising:
a surface feature comprising one or more charged linker molecules; and
a charged carbon nanotube controllably deposited on said charged linker molecules, wherein the charge of the charged carbon nanotube is opposite the charge of the charged linker molecules.

30. A device comprising the substrate of claim 29.

31. An electronic circuit, comprising the substrate of claim 29.

32. A molecular photon emitter comprising the substrate of claim 29.

33. A sensor comprising the substrate of claim 29.

34. A molecular electronic circuit comprising the substrate of claim 29.

35. The substrate of claim 29, further comprising a surfactant bound to said carbon nanotube.

36. The substrate of claim 35, further comprising a macromolecule bound to said surfactant.

37. The substrate of claim 36, wherein said macromolecule is a nucleic acid or a protein.

38. The substrate of claim 29, further comprising a microfluidic channel adjacently positioned to said surface feature.

39. The substrate of claim 29, wherein said surface feature is a channel having a width smaller than about 1000 nm.

40. A process, comprising:
providing an aqueous carbon nanotube dispersion comprising water and individual, dispersed, carbon nanotubes; and
chromatographically separating said carbon nanotubes.

41. The process of claim 40, further comprising sequentially removing elutes of the separated carbon nanotubes.

42. Carbon nanotubes made by the process of claim 41.

43. The carbon nanotubes of claim 42, wherein the chromatographically separated carbon nanotubes have a narrower polydispersity than the carbon nanotubes provided in the aqueous carbon nanotube dispersion.

44. A monodisperse carbon nanotube dispersion made by the process of claim 41.

45. The process of claim 40, wherein the carbon nanotubes comprise SWNTs.

46. A device, comprising:
a substrate fluidically sealed to a microfluidic assembly, said substrate comprising charged carbon nanotubes adsorbed on one or more charged regions on a surface of the substrate, wherein the charge of the charged carbon nanotubes is opposite the charge of the charged regions
said microfluidic assembly comprising
one or more contacting regions adjacently positioned to the substrate for controllably contacting one or more molecular components to said carbon nanotubes;
one or more target fluid conduits capable of supplying one or more target fluids comprising one or more analytes;
one or more detecting molecule conduits capable of supplying one or more detecting molecules for detecting said analytes in the target fluids;
one or more valves capable of directing said target fluids and said detecting molecules into said contacting regions; and
optionally one or more exit conduits.

47. The device of claim 46, wherein the charged carbon nanotubes are negatively charged and the charged regions are positively charged.

48. The device of claim 46, wherein the detecting molecules comprise one or more antibodies and the analytes comprise one or more proteins.
